(19) United States
(12) Patent Application Publication
(10) Pub. No.: US 2011/0028531 A1
(43) Pub. Date: Feb. 3, 2011

(54) NOVEL SIRNA COMPOUNDS FOR INHIBITING RTP801

(76) Inventors: Elena Feinstein, Rehovot (IL); Rami Skaliter, Nes Ziona (IL); Hagar Kalinski, Rishon-le-Zion (IL); Igor Mett, Rehovot (IL)

Correspondence Address: COOPER & DUNHAM, LLP, 30 Rockefeller Plaza, 20th Floor, NEW YORK, NY, 10112, US

(21) Appl. No.: 12/736,230

(22) PCT Filed: Mar. 17, 2009
(86) PCT No.: PCT/IL09/00302

§ 371 Date: Oct. 20, 2010

Related U.S. Application Data
(60) Provisional application No. 61/070,181, filed on Mar. 20, 2008.

(51) Int. Cl. A61K 31/713; C07H 21/02; A61P 25/28; A61P 13/12; A61P 11/00; A61P 27/02; A61P 9/10; A61P 27/16; A61P 25/00; A61P 25/16; A61P 11/06; A61P 27/06

(52) U.S. Cl. 514/44 A; 536/24.5

(57) ABSTRACT

The present invention provides chemically modified siRNA compounds that target RTP801 and pharmaceutical compositions comprising same useful for treating microvascular disorders, eye diseases, hearing impairment, neurodegenerative diseases and disorders, spinal cord injury and respiratory conditions.

This application claims priority from U.S. Provisional Patent Application Ser. No. 61/070,181, which is hereby incorporated by reference in its entirety.

### FIELD OF THE INVENTION

The present invention relates to novel siRNA oligonucleotides and to chemically modified siRNA compounds which inhibit RTP801 and to the use of the compounds to treat respiratory disorders (including pulmonary disorders), eye diseases and conditions, hearing impairments (including hearing loss), neurodegenerative disorders, spinal cord injury, microvascular disorders, angiogenesis- and apoptosis-related conditions.

### BACKGROUND OF THE INVENTION

### RTP801

The RTP801 gene was first reported by the assignee of the instant application. U.S. Pat. Nos. 6,455,674, 6,555,667, and 6,740,738, and related patents to the assignee of the instant application and hereby incorporated by reference in their entirety, disclose the RTP801 polynucleotide and polypeptide, and antibodies directed toward the polypeptide. RTP801 represents a unique gene target for hypoxia-inducible factor-1 (HIF-1) that may regulate hypoxia-induced pathogenesis independently of growth factors such as VEGF. PCT Patent Application Nos. PCT/US2005/029236, PCT/US2007/001468 and PCT/US2008/002483, to the assignee of the instant application and hereby incorporated by reference in their entirety, relate to compounds including siRNA, for inhibition of RTP801.

The assignee of the instant application has discovered a similar, albeit distinct, gene termed RTP801L (for RTP801-like) which can be used in combination therapies with RTP801 (see below). For further information concerning RTP801L see PCT Publication No. WO 2007/141796, assigned to the assignee of the instant application, which is hereby incorporated by reference in its entirety.

The following patents and patent applications give aspects of background information: WO 2001/070979, US 2003108871, US 2002119463, WO 2004/018999, EP 1394274, WO 2002/101075, WO 2003/010205 and WO 2002/046465. The following publications give background information: Shoshani, et al. Mol. Cel. Biol., April 2002, p. 2283-2293; Brafman, et al. Invest Opthalmol Vis Sci. 2004. 45(10):3796-805; Ellisen, et al. Molecular Cell, 2002. 10:995-1005; and Richard et al. J. Biol. Chem. 2000, 275(35): 26765-71.

siRNA and RNA Interference

RNA interference (RNAi) is a phenomenon involving double-stranded (ds) RNA-dependent gene-specific posttranscriptional silencing. Initial attempts to study this phenomenon and to manipulate mammalian cells experimentally were frustrated by an active, non-specific antiviral defense mechanism which was activated in response to long dsRNA molecules (Gil et al., Apoptosis, 2000. 5:107-114). Later, it was discovered that synthetic duplexes of 21 nucleotide RNAs could mediate gene specific RNAi in mammalian cells, without stimulating the generic antiviral defense mechanisms (Elbashir et al. Nature 2001, 411:494-498 and Caplen et al. PNAS 2001, 98:9742-9747). As a result, small interfering RNAs (siRNAs), which are short double-stranded RNAs, have been widely used to inhibit gene expression and understand gene function.

RNA interference (RNAi) is mediated by small interfering RNAs (siRNAs) (Fire et al, Nature 1998, 391:806) or microRNAs (miRNAs) (Ambros V. Nature 2004, 431:350-355 and Bartel D P. Cell. 2004 116(2):281-97). The corresponding process in plants is commonly referred to as specific post-transcriptional gene silencing and as quelling in fungi.

A siRNA is a double-stranded RNA (dsRNA) which down-regulates or silences (i.e. fully or partially inhibits) the expression of an endogenous or exogenous gene/mRNA. RNA interference is based on the ability of certain dsRNA species to enter a specific protein complex, where they are then targeted to complementary cellular RNA (i.e. mRNA), which they specifically degrade or cleave. Thus, the RNA interference response features an endonuclease complex containing siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir, et al., Genes Dev., 2001, 15:188). In more detail, longer dsRNAs are digested into short (17-29 bp) dsRNA fragments (also referred to as short inhibitory RNAs or “siRNAs”) by type III RNAses (DICER, DROSHA, etc., see Bernstein et al., Nature, 2001, 409:363-6 and Lee et al., Nature, 2003, 425:415-9). The RISC protein complex recognizes these fragments and complementary mRNA. The whole process is culminated by endonuclease cleavage of target mRNA (McManus and Sharp, Nature Rev Genet, 2002, 3:737-47; Paddison and Hannon, Curr Opin Mol. Ther. 2003, 5(3): 217-24). For additional information on these terms and proposed mechanisms, see for example, Bernstein, et al., RNA. 2001, 7(11):1509-21; Nishikura, Cell. 2001, 107(4):415-8 and PCT Publication No. WO 01/36646.

Studies have revealed that siRNA can be effective in vivo in mammals including humans. Specifically, Bitko et al., showed that specific siRNAs directed against the respiratory syncytial virus (RSV) nucleocapsid N gene are effective in treating mice when administered intranasally (Nat. Med. 2005, 11(1):50-55). For reviews of therapeutic applications of siRNAs see for example Batik (Mol. Med. 2005, 83: 764-773) and Chakraborty (Current Drug Targets 2007 8(3):469-82). In addition, clinical studies with short siRNAs that target the VEGF receptor 1 (VEGFR1) to treat age-related macular degeneration (AMD) have been conducted in human patients (Kaiser, Am J. Opthalmol. 2006 142(4):660-8). Further information on the use of siRNA as therapeutic agents is found in Durcan, 2008. Mol. Pharma. 5(4):559-566; Kim and Rossi, 2008. BioTechniques 44:613-616; Grimm and Kay, 2007, JCI, 117(12):3633-41.

Chemically Modified siRNA

The selection and synthesis of siRNA corresponding to known genes has been widely reported; (see for example Ui-Tei et al., 2006. J Biomed Biotechnol. 2006:65052; Chalk et al., 2004. BBRC. 319(1): 264-74; Sioud & Leirdal, 2004. Met. Mol. Biol. 252:457-69; Levenkova et al., 2004, Bioinform. 20(3):430-2; Ui-Tei et al., 2004. NAR 32(3):936-48).

Examples for the use of, and production of, modified siRNA are found in Braasch et al., 2003. Biochem., 42(26):7967-75; Chiu et al., 2003, RNA, 9(9):1034-48; PCT publications WO 2004/015107 (atugen AG) and WO 02/44321 (Tuschl et al). U.S. Pat. Nos. 5,898,031 and 6,107,094 teach chemically modified oligomers. U.S. Pat. No. 7,452,987 relates to oligomeric compounds having alternating unmodified and 2′ sugar modified ribonucleotides. US patent publication No. 2005/0042647 describes dsRNA compounds having chemically modified internucleoside linkages.

The inclusion of a 5′-phosphate moiety was shown to enhance activity of siRNAs in Drosophila embryos (Boutla, et al., 2001, Curr. Biol. 11:1776-1780) and is required for siRNA function in human HeLa cells (Schwarz et al., 2002, Mol. Cell, 10:537-548).

Amarzguoui et al., (2003, NAR, 31(2):589-595) showed that siRNA activity depended on the positioning of the 2′-O-methyl modifications. Holen et al (2003, NAR, 31(9):2401-2407) report that an siRNA having small numbers of 2′-O-methyl modified nucleosides showed good activity compared to wild type but that the activity decreased as the numbers of 2′-O-methyl modified nucleosides was increased. Chiu and Rana (2003, RNA, 9:1034-1048) teach that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czauderna et al., 2003, NAR, 31(11), 2705-2716).

PCT Patent Application Nos. PCT/IL2008/000248 and PCT/IL2008/001197, assigned to the assignee of the present invention, and hereby incorporated by reference in their entirety, disclose motifs useful in the preparation of chemically modified siRNA compounds.

Respiratory disorders of all types (including pulmonary disorders), eye diseases and conditions, hearing impairments (including hearing loss), microvascular disorders, neurodegenerative diseases and disorders, spinal cord injury, angiogenesis- and apoptosis-related conditions affect millions of people worldwide. There is a need to identify new drugs and new drug targets useful in treating subjects suffering from or susceptible to these diseases and disorders.

Stable and active siRNA compounds which inhibit the RTP801 gene and that are useful in treating the above mentioned diseases and disorders would be of great therapeutic value.

### SUMMARY OF THE INVENTION

The present invention provides, in one aspect, novel double stranded chemically modified oligonucleotides that inhibit or reduce expression of the RTP801 target gene. The oligonucleotides are useful in the preparation of pharmaceutical compositions for treating subjects suffering from microvascular disorders, eye diseases and conditions (e.g. macular degeneration), hearing impairments (including hearing loss), respiratory disorders, neurodegenerative disorders, spinal cord injury, angiogenesis- and apoptosis-related conditions.

In one aspect the present invention provides novel siRNA molecules, which inhibit the RTP801 gene and can be used to treat various diseases and indications.

Accordingly, in one aspect the present invention provides a siRNA compound having the following structure:

| | |
|---|---|
| 5′ (N)ₓ-Z 3′ | (antisense strand) |
| 3′ Z′-(N′)_{y}-z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
each of x and y is independently an integer between 18 and 40;
wherein the sequence of (N′)y is substantially complementary to the sequence of (N)x;
and wherein (N)x comprises an antisense that is substantially complementary to the RTP801 mRNA.

In some embodiments the compound comprises a phosphodiester bond. In preferred embodiments (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide and (N′)y comprises at least one mirror nucleotide, or a nucleotide joined to an adjacent nucleotide by a 2′-5′ internucleotide phosphate bond.

In additional embodiments (N)x comprises modified ribonucleotides in alternating positions wherein each N at the 5′ and 3′ termini are modified in their sugar residues and the middle ribonucleotide is not modified, e.g. ribonucleotide in position 10 in a 19-mer strand.

For all the structures, in some embodiments the covalent bond joining each consecutive N or N′ is a phosphodiester bond. In various embodiments all the covalent bonds are phosphodiester bonds.

In various embodiments x=y and each of x and y is 19, 20, 21, 22 or 23. In some embodiments x=y=21. In other embodiments x=y=19.

In one embodiment of the above structure, the compound comprises at least one mirror nucleotide at one terminus or both termini in (N′)y. In various embodiments the compound comprises two consecutive mirror nucleotides, one at the 3′ penultimate position and one at the 3′ terminus in (N′)y. In one preferred embodiment x=y=19 and (N′)y comprises an L-deoxyribonucleotide at position 18.

In some embodiments the mirror nucleotide is selected from an L-ribonucleotide and an L-deoxyribonucleotide. In various embodiments the mirror nucleotide is an L-deoxyribonucleotide. In some embodiments y=19 and (N′)y, consists of unmodified ribonucleotides at positions 1-17 and 19 and one L-DNA at the 3′ penultimate position (position 18). In other embodiments y=19 and (N′)y consists of unmodified ribonucleotides at position 1-16 and 19 and two consecutive L-DNA at the 3′ penultimate position (positions 17 and 18).

In some embodiments (N)x and its corresponding sense strand (N′)y are selected from any one of the oligonucleotide pairs shown in Tables A-I, set forth in SEQ ID NOS:3-3624. In certain embodiments the nucleotide sequence of (N)x is set forth in any one of SEQ ID NO:16 and SEQ ID NO:1243.

In some embodiments in (N)x the ribonucleotides alternate between 2′-O-Methyl sugar modified ribonucleotides and unmodified ribonucleotides and the ribonucleotide located at the middle of (N)x being unmodified.

In some embodiments (N)x comprises at least five alternating unmodified ribonucleotides and 2′O methyl sugar modified ribonucleotides beginning at the 3′ end and at least nine 2′O methyl sugar modified ribonucleotides in total and each remaining N is an unmodified ribonucleotide.

In some embodiments in (N)x 1-5 consecutive N at the 5′ terminus are 2′O Methyl sugar modified ribonucleotides and the remainder of the N are unmodified ribonucleotides.

In another embodiment of the above structure, (N′)y further comprises one or more nucleotides containing a sugar moiety modified with an extra bridge at one or both termini. Non-limiting examples of such nucleotides, also referred to herein as bicyclic nucleotides, are locked nucleic acid (LNA) and ethylene-bridged nucleic acid (ENA).

In another embodiment of the above structure, (N′)y comprises at least two consecutive nucleotide joined together to the next nucleotide by a 2′-5′ phosphodiester bond at one or both termini. In certain preferred embodiments in (N′)y the 3′ penultimate nucleotide is linked to the 3′ terminal nucleotide with a 2′-5′ phosphodiester bridge.

In certain preferred embodiments the compound of the invention is a blunt-ended (z″, Z and Z′ are absent), double stranded oligonucleotide structure, x=y and x=19 or 23, wherein (N′)y comprises unmodified ribonucleotides in which three consecutive nucleotides at the 3′ terminus are joined together by two 2′-5′ phosphodiester bonds; and an antisense strand (AS) of alternating unmodified and 2′-O methyl sugar-modified ribonucleotides.

In some embodiments, neither (N)x nor (N′)y are phosphorylated at the 3′ and 5′ termini. In other embodiments either or both (N)x and (N′)y are phosphorylated at the 3′ termini.

In certain embodiments for all the above-mentioned structures, the compound is blunt ended, for example wherein both Z and Z′ are absent. In an alternative embodiment, the compound comprises at least one 3′ overhang and or a 5′ capping moiety at the 5′ terminus of (N′)y, wherein at least one of Z or Z′ or z″ is present. Z, Z′ and z″ are independently one or more covalently linked modified or non-modified nucleotides, for example inverted dT or dA; dT, LNA, mirror nucleotide and the like. In some embodiments each of Z and Z′ are independently selected from dT and dTdT. In certain specific embodiments Z and Z′ are absent, z″ is present and consists of inverted deoxyabasic moiety.

In some embodiments the siRNA sense and antisense oligonucleotides are selected from sense and corresponding antisense oligonucleotides listed in any one of Tables A-I, set forth in any one of SEQ ID NOS:3-3624.

In a second aspect the present invention provides a pharmaceutical composition comprising one or more compounds of the present invention, in an amount effective to inhibit target gene expression, and a pharmaceutically acceptable carrier wherein the target gene is RTP801.

In another aspect, the present invention relates to a method for the treatment of a subject in need of treatment for a disease or disorder or symptom or condition associated with the disease or disorder, associated with the expression of RTP801 comprising administering to the subject an amount of an siRNA which reduces or inhibits expression of RTP801. In preferred embodiments the siRNA compound is chemically modified according to the embodiments of the present invention.

In some embodiments the present invention provides a method of treating a subject suffering from, inter alia, a microvascular disorder, an eye disease or disorder, a hearing impairment (including hearing loss), a respiratory (including pulmonary) disorder, a neurodegenerative disease or disorder, a spinal cord injury, angiogenesis- and apoptosis-related conditions comprising administering to the subject a pharmaceutical composition comprising at least one RTP801 inhibitor.

In one embodiment the respiratory disorder is chronic obstructive pulmonary disease (COPD). Thus, the present invention provides a method of treating a subject suffering from COPD, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in promoting recovery in a subject suffering from a respiratory disorder.

In another embodiment the eye disorder is macular degeneration. Thus, the present invention provides a method of treating a subject suffering from macular degeneration, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in promoting recovery in a subject suffering from macular degeneration. In one embodiment the macular degeneration is age related macular degeneration (AMD).

In another embodiment the present invention provides a method of treating a subject suffering from a microvascular disorder, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in promoting recovery in a subject suffering from a microvascular disorder. In one embodiment the microvascular disorder is diabetic retinopathy.

In another embodiment the present invention provides a method of treating a subject suffering from a hearing impairment, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in promoting recovery in a subject suffering from a hearing impairment. In one embodiment the hearing impairment is hearing loss.

In a further embodiment the present invention provides a method of treating a subject suffering from a spinal cord injury, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in promoting recovery in a subject suffering from a spinal cord injury.

In a further embodiment the present invention provides a method of treating a subject suffering from a neurodegenerative disease or disorder, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in stabilizing cognitive function at the level existing at time of diagnosis in a subject suffering from a neurodegenerative disease or disorder. In certain embodiments inhibition of RTP801 gene by at least one chemically modified siRNA molecule of the invention is effective in stabilizing motor function at the level existing at time of diagnosis in a subject suffering from a neurodegenerative disease or disorder. In certain embodiments inhibition of RTP801 gene by at least one chemically modified siRNA molecule of the invention is effective in promoting recovery in a subject suffering from a neurodegenerative disease or disorder. In certain embodiments inhibition of RTP801 gene by at least one chemically modified siRNA molecule of the invention is effective in slowing the progress of neurodegenerative disease or disorder in a subject suffering from a neurodegenerative disease or disorder.

In a further embodiment the present invention provides a method of treating a subject suffering from an angiogenesis-related condition, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in promoting recovery in a subject suffering from an angiogenesis-related condition.

In a further embodiment the present invention provides a method of treating a subject suffering from an apoptosis-related condition, comprising administering to the subject a pharmaceutical composition comprising a therapeutically effective amount of at least one chemically modified siRNA which inhibits the expression of the RTP801 gene. In certain embodiments inhibition of RTP801 gene by at least one chemically modified siRNA molecule of the invention is effective in promoting recovery in a subject suffering from an apoptosis-related condition.

The present invention provides novel structures of double stranded chemically modified oligonucleotides, having advantageous properties and which are applicable to siRNA to any target sequence, particularly the mRNA sequences of the RTP801 gene, to down-regulate the expression of the RTP801 gene by the mechanism of RNA interference. The invention also provides a pharmaceutical composition comprising at least one chemically modified siRNA molecule of the invention and methods of using the same in therapeutic applications.

The present invention explicitly excludes known chemically modified siRNA compounds.

The preferred methods, materials, and examples that will now be described are illustrative only and are not intended to be limiting; materials and methods similar or equivalent to those described herein can be used in practice or testing of the invention. Other features and advantages of the invention will be apparent from the following detailed description, and from the claims.

### DETAILED DESCRIPTION OF THE INVENTION

The present invention, in some of its embodiments, provides chemically modified siRNA compounds, pharmaceutical compositions comprising at least one compound of the invention and methods for alleviation or reduction of the symptoms and signs associated with eye diseases, respiratory disorders, neurodegenerative disorders, spinal cord injury, hearing impairments and microvascular disorders, inter alia.

Without being bound by theory, the inventors of the present invention have found that RTP801 is involved in various disease states and disorders including, without being limited to, microvascular disorders, eye diseases, neurodegenerative disease and disorders, respiratory disorders, hearing impairments, angiogenesis- and apoptosis-related conditions and spinal cord injury and disease, and it would be beneficial to inhibit RTP801 in order to treat any of the above mentioned diseases and disorders. Methods, siRNA molecules and compositions which inhibit RTP801 are discussed herein at length, and any of said molecules and/or compositions may be beneficially employed in the treatment of a subject suffering from or susceptible to any of said conditions.

Accordingly, in certain aspects the present invention provides chemically modified siRNA compounds and pharmaceutical compositions comprising same useful in inhibiting expression of the RTP801 gene in vivo.

In another aspect, the present invention provides a method of treating a subject suffering from or susceptible to a microvascular disorder, eye disease or disorder, hearing impairment (including hearing loss), a respiratory (including pulmonary) disorder, neurodegenerative disease or disorder, spinal cord injury, angiogenesis- and apoptosis-related conditions, comprising administering to the subject a pharmaceutical composition comprising at least one chemically modified small interfering RNA (i.e., siRNA) of the invention that is targeted to a RTP801 mRNA and hybridize to it, in an amount sufficient to down-regulate expression of RTP801 gene by an RNA interference mechanism.

In certain embodiments, the subject compounds are useful in inhibiting expression of the RTP801 gene for treatment of respiratory disorders, microvascular disorders or eye disorders. Particular diseases and conditions to be treated are ARDS; COPD; ALI; Emphysema; Diabetic Neuropathy, nephropathy and retinopathy; DME and other diabetic conditions; Glaucoma; AMD; BMT retinopathy; ischemic conditions including stroke; OIS; neurodegenerative disorders such as Parkinson's, Alzheimer's, ALS; kidney disorders: ARF, DGF, transplant rejection; hearing disorders; spinal cord injuries; oral mucositis; dry eye syndrome and pressure sores.

In various embodiments the present invention provides a method of treating a subject suffering from a microvascular disorder, an eye disease or a respiratory disorder, comprising administering to the subject a pharmaceutical composition comprising at least one chemically modified siRNA molecule according to the present invention in a therapeutically effective amount so as to thereby treat the subject.

In various embodiments the method comprises administering to the subject a pharmaceutical composition comprising a therapeutically effective dose of at least one chemically modified siRNA molecule according to the present invention which targets the RTP801 gene in a dosage and over a period of time so as to thereby treat the patient.

The invention further provides a method of treating a subject suffering from a microvascular disorder, an eye disease, a neurodegenerative disease, spinal cord injury, hearing impairment or a respiratory disorder, comprising administering to the subject a pharmaceutical composition comprising at least one chemically modified siRNA molecule according to the present invention, in a dosage and over a period of time sufficient to promote recovery of the subject. The eye disease include macular degeneration such as age-related macular degeneration (AMD), inter alia. The microvascular disorder includes diabetic retinopathy and acute renal failure, inter alia. The respiratory disorder includes chronic obstructive pulmonary disease (COPD), emphysema, chronic bronchitis, asthma and lung cancer, inter alia. The neurodegenerative disorder includes Alzheimer's disease, Parkinson's disease, ALS, inter alia. In various embodiments the chemically modified siRNA compounds of the invention comprise sense and antisense oligonucleotides that are selected from sense and corresponding antisense oligonucleotides presented in any one of Tables A-I, set forth in any one of SEQ ID NOS:3-3624.

Accordingly, the present invention further provides a method of treating a subject suffering from or susceptible to macular degeneration comprising administering to the subject a pharmaceutical composition comprising a therapeutically effective amount of at least one chemically modified siRNA according to the present invention, wherein the chemically modified siRNA attenuates expression of the RTP801 gene so as to thereby treat the patient. In various embodiments the at least one siRNA comprises consecutive nucleotides having a sequence identical to any one of the sequences set forth in Tables A-I (SEQ ID NOs:3-3624).

The present invention further provides a method of treating a subject suffering from or susceptible to COPD, comprising administering to the subject a pharmaceutical composition comprising a therapeutically effective amount of at least one chemically modified siRNA according to the present invention, wherein the chemically modified siRNA attenuates expression of the RTP801 gene so as to thereby treat the patient. In various embodiments the at least one siRNA sense and antisense strands are selected form any one of the sequencers in Tables A-I, set forth SEQ ID NOs:3-3624.

The present invention further provides a method of treating a subject suffering from or susceptible diabetic retinopathy, comprising administering to the subject a pharmaceutical composition comprising a therapeutically effective amount of at least one chemically modified siRNA according to the present invention, wherein the chemically modified siRNA attenuates expression of the RTP801 gene so as to thereby treat the patient. In various embodiments the at least one siRNA sense and antisense strands are selected form any one of the sequencers in Tables A-I, set forth SEQ ID NOs:3-3624.

In various embodiments the neurodegenerative disorder is selected from neurodegenerative conditions causing problems with movements, such as ataxia; and conditions affecting memory and related to dementia. In various embodiments the neurodegenerative disorder is selected from Parkinson's disease, ALS (Lou Gehrig's Disease), Alzheimer's disease, Lewy body dementia, Huntington's disease and any other disease-induced dementia (such as HIV-associated dementia for example).

In further embodiments, this invention provides novel chemically modified siRNA compounds, pharmaceutical compositions comprising them and methods for alleviation or reduction of symptoms and signs associated with neurological disorders arising from ischemic or hypoxic conditions. Non-limiting examples of such conditions are hypertension, hypertensive cerebral vascular disease, a constriction or obstruction of a blood vessel—as occurs in the case of a thrombus or embolus, angioma, blood dyscrasias, any form of compromised cardiac function including cardiac arrest or failure, systemic hypotension. In one embodiment the neurological disorder is stroke. In another embodiment the neurological disorder is epilepsy.

Lists of preferred siRNA compounds are provided in Tables A-I. The separate lists of 19-mer, 21-mer and 23-mer siRNAs are prioritized based on their score according to a proprietary algorithm as the best sequences for targeting the human gene expression.

Methods, molecules and compositions, which inhibit target genes are discussed herein at length, and any of said molecules and/or compositions are beneficially employed in the treatment of a patient suffering from any of said conditions. Tables A, B, D, E and I set forth 19-mer oligomers. Tables C and F set forth 21-mer oligomers. Tables G and H set forth 23-mer oligomers.

### DEFINITIONS

For convenience certain terms employed in the specification, examples and claims are described herein.

It is to be noted that, as used herein, the singular forms “a”, “an” and “the” include plural forms unless the content clearly dictates otherwise.

Where aspects or embodiments of the invention are described in terms of Markush groups or other grouping of alternatives, those skilled in the art will recognize that the invention is also thereby described in terms of any individual member or subgroup of members of the group.

An “inhibitor” is a compound, which is capable of reducing (partially or fully) the expression of a gene or the activity of the product of such gene to an extent sufficient to achieve a desired biological or physiological effect. The term “inhibitor” as used herein refers to a siRNA inhibitor.

A “siRNA inhibitor” is a compound which is capable of reducing the expression of a gene or the activity of the product of such gene to an extent sufficient to achieve a desired biological or physiological effect. The term “siRNA inhibitor” as used herein refers to one or more of a siRNA, shRNA, synthetic shRNA; miRNA. Inhibition may also be referred to as down-regulation or, for RNAi, silencing.

The term “inhibit” as used herein refers to reducing the expression of a gene or the activity of the product of such gene to an extent sufficient to achieve a desired biological or physiological effect. Inhibition is either complete or partial.

As used herein, the term “inhibition” of a target gene means inhibition of the gene expression (transcription or translation) or polypeptide activity of a target gene wherein the target gene is RTP801 or variants thereof. The polynucleotide sequence of the target mRNA sequence, or the target gene having a mRNA sequence refer to the mRNA sequence or any homologous sequences thereof preferably having at least 70% identity, more preferably 80% identity, even more preferably 90% or 95% identity to the mRNA of RTP801. Therefore, polynucleotide sequences derived from the RTP801 mRNA which have undergone mutations, alterations or modifications as described herein are encompassed in the present invention. The terms “mRNA polynucleotide sequence”, “mRNA sequence” and “mRNA” are used interchangeably.

As used herein, the terms “polynucleotide” and “nucleic acid” may be used interchangeably and refer to nucleotide sequences comprising deoxyribonucleic acid (DNA), and ribonucleic acid (RNA). The terms are to be understood to include, as equivalents, analogs of either RNA or DNA made from nucleotide analogs. Throughout this application, mRNA sequences are set forth as representing the corresponding genes.

“Oligonucleotide” or “oligomer” refers to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The compounds of the present invention encompass molecules comprising deoxyribonucleotides, ribonucleotides, modified deoxyribonucleotides, modified ribonucleotides and combinations thereof.

Substantially complementary refers to complementarily of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

“Nucleotide” is meant to encompass deoxyribonucleotides and ribonucleotides, which may be natural or synthetic, and or modified or unmodified. Modifications include changes to the sugar moiety, the base moiety and or the linkages between ribonucleotides in the oligoribonucleotide. As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide.

The nucleotides can be selected from naturally occurring or synthetic modified bases. Naturally occurring bases include adenine, guanine, cytosine, thymine and uracil.

Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine. In some embodiments one or more nucleotides in an oligomer is substituted with inosine.

According to some embodiments the present invention provides inhibitory oligonucleotide compounds comprising unmodified and modified nucleotides and or unconventional moieties. The compound comprises at least one modified nucleotide selected from the group consisting of a sugar modification, a base modification and an internucleotide linkage modification and may contain DNA, and modified nucleotides such as LNA (locked nucleic acid), ENA (ethylene-bridged nucleic acid), PNA (peptide nucleic acid), arabinoside, phosphonocarboxylate or phosphinocarboxylate nucleotide (PACE nucleotide), mirror nucleotide, or nucleotides with a 6 carbon sugar.

All analogs of, or modifications to, a nucleotide/oligonucleotide are employed with the present invention, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

A sugar modification includes a modification on the 2′ moiety of the sugar residue and encompasses amino, fluoro, alkoxy e.g. methoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O-, S-, or N-alkyl; O-, S, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1.

In one embodiment the siRNA compound comprises at least one ribonucleotide comprising a 2′ modification on the sugar moiety (“2′ sugar modification”). In certain embodiments the compound comprises 2′O-alkyl or 2′-fluoro or 2′O-allyl or any other 2′ modification, optionally on alternate positions. Other stabilizing modifications are also possible (e.g. terminal modifications). In some embodiments a preferred 2′O-alkyl is 2′O-methyl (methoxy) sugar modification.

In some embodiments the backbone of the oligonucleotides is modified and comprises phosphate-D-ribose entities but may also contain thiophosphate-D-ribose entities, triester, thioate, 2′-5′ bridged backbone (also may be referred to as 5′-2′), PACE and the like.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments it is a deoxyribonucleotide. In addition, analogues of polynucleotides may be prepared wherein the structure of one or more nucleotide is fundamentally altered and better suited as therapeutic or experimental reagents. An example of a nucleotide analogue is a peptide nucleic acid (PNA) wherein the deoxyribose (or ribose) phosphate backbone in DNA (or RNA is replaced with a polyimide backbone which is similar to that found in peptides. PNA analogues have been shown to be resistant to enzymatic degradation and to have enhanced stability in vivo and in vitro. Other modifications that can be made to oligonucleotides include polymer backbones, cyclic backbones, acyclic backbones, thiophosphate-D-ribose backbones, triester backbones, thioate backbones, 2′-5′ bridged backbone, artificial nucleic acids, morpholino nucleic acids, glycol nucleic acid (GNA), threose nucleic acid (TNA), arabinoside, and mirror nucleoside (for example, beta-L-deoxyribonucleoside instead of beta-D-deoxyribonucleoside). Examples of siRNA compounds comprising LNA nucleotides are disclosed in Elmen et al., (NAR 2005, 33(1):439-447).

The compounds of the present invention can be synthesized using one or more inverted nucleotides, for example inverted thymidine or inverted adenine (see, for example, Takei, et al., 2002, JBC 277(26):23800-06).

Other modifications include terminal modifications on the 5′ and/or 3′ part of the oligonucleotides and are also known as capping moieties. Such terminal modifications are selected from a nucleotide, a modified nucleotide, a lipid, a peptide, a sugar and inverted abasic moiety.

What is sometimes referred to in the present invention as an “abasic nucleotide” or “abasic nucleotide analog” is more properly referred to as a pseudo-nucleotide or an unconventional moiety. A nucleotide is a monomeric unit of nucleic acid, consisting of a ribose or deoxyribose sugar, a phosphate, and a base (adenine, guanine, thymine, or cytosine in DNA; adenine, guanine, uracil, or cytosine in RNA). A modified nucleotide comprises a modification in one or more of the sugar, phosphate and or base. The abasic pseudo-nucleotide lacks a base, and thus is not strictly a nucleotide.

The term “capping moiety” as used herein includes abasic ribose moiety, abasic deoxyribose moiety, modifications abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′O-Me nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Certain preferred capping moieties are abasic ribose or abasic deoxyribose moieties; inverted abasic ribose or abasic deoxyribose moieties; C6-amino-Pi; a mirror nucleotide including L-DNA and L-RNA.

The term “unconventional moiety” as used herein refers to abasic ribose moiety, an abasic deoxyribose moiety, a deoxyribonucleotide, a modified deoxyribonucleotide, a minor nucleotide, a non-base pairing nucleotide analog and a nucleotide joined to an adjacent nucleotide by a 2′-5′ internucleotide phosphate bond; bridged nucleic acids including LNA and ethylene bridged nucleic acids.

Abasic deoxyribose moiety includes for example abasic deoxyribose-3′-phosphate; 1,2-dideoxy-D-ribofuranose-3-phosphate; 1,4-anhydro-2-deoxy-D-ribitol-3-phosphate. Inverted abasic deoxyribose moiety includes inverted deoxyriboabasic; 3′,5′ inverted deoxyabasic 5′-phosphate.

A “mirror” nucleotide is a nucleotide with reversed chirality to the naturally occurring or commonly employed nucleotide, i.e., a mirror image (L-nucleotide) of the naturally occurring (D-nucleotide), also referred to as L-RNA in the case of a mirror ribonucleotide, and “spiegelmer”. The nucleotide can be a ribonucleotide or a deoxyribonucleotide and my further comprise at least one sugar, base and or backbone modification. See U.S. Pat. No. 6,586,238. Also, U.S. Pat. No. 6,602,858 discloses nucleic acid catalysts comprising at least one L-nucleotide substitution. Mirror nucleotide includes for example L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboadenosine-3′-phosphate (mirror rA); L-ribocytidine-3′-phosphate (mirror rC); L-riboguanosine-3′-phosphate (mirror rG); L-ribouracil-3′-phosphate (mirror dU).

Modified deoxyribonucleotide includes, for example 5′OMe DNA (5-methyl-deoxyriboguanosine-3′-phosphate) which may be useful as a nucleotide in the 5′ terminal position (position number 1); PACE (deoxyriboadenine 3′ phosphonoacetate, deoxyribocytidine 3′ phosphonoacetate, deoxyriboguanosine 3′ phosphonoacetate, deoxyribothymidine 3′ phosphonoacetate.

Bridged nucleic acids include LNA (2′-O, 4′-C-methylene bridged Nucleic Acid adenosine 3′ monophosphate, 2′-O,4′-C-methylene bridged Nucleic Acid 5-methyl-cytidine 3′ monophosphate, 2′-O,4′-C-methylene bridged Nucleic Acid guanosine 3′ monophosphate, 5-methyl-uridine (or thymidine) 3′ monophosphate); and ENA (2′-O,4′-C-ethylene bridged Nucleic Acid adenosine 3′ monophosphate, 2′-O,4′-C-ethylene bridged Nucleic Acid 5-methyl-cytidine 3′ monophosphate, 2′-O,4′-C-ethylene bridged Nucleic Acid guanosine 3′ monophosphate, 5-methyl-uridine (or thymidine) 3′ monophosphate).

In some embodiments of the present invention a preferred unconventional moiety is an abasic ribose moiety, an abasic deoxyribose moiety, a deoxyribonucleotide, a mirror nucleotide, and a nucleotide joined to an adjacent nucleotide by a 2′-5′ internucleotide phosphate bond.

According to one aspect the present invention provides inhibitory oligonucleotide compounds comprising unmodified and modified nucleotides. The compound comprises at least one modified nucleotide selected from the group consisting of a sugar modification, a base modification and an internucleotide linkage modification and may contain DNA, and modified nucleotides such as LNA (locked nucleic acid) including ENA (ethylene-bridged nucleic acid; PNA (peptide nucleic acid); arabinoside; PACE (phosphonoacetate and derivatives thereof), mirror nucleotide, or nucleotides with a six-carbon sugar.

“RTP801 gene” refers to the RTP801 coding sequence open reading frame, set forth in SEQ ID NO:1, or any homologous sequence thereof preferably having at least 70% identity, more preferable 80% identity, even more preferably 90% or 95% identity. This encompasses any sequences derived from SEQ ID NO:1 which have undergone mutations, alterations or modifications as described herein. Thus, in a preferred embodiment RTP801 is encoded by a nucleic acid sequence according to SEQ ID NO 1. It is also within the present invention that the nucleic acids according to the present invention are only complementary and identical, respectively, to a part of the nucleic acid coding for RTP801 as, preferably, the first stretch and first strand is typically shorter than the nucleic acid according to the present invention. It is also to be acknowledged that based on the amino acid sequence of RTP801 any nucleic acid sequence coding for such amino acid sequence can be perceived by the one skilled in the art based on the genetic code. However, due to the assumed mode of action of the nucleic acids according to the present invention, it is most preferred that the nucleic acid coding for RTP801, preferably the mRNA thereof, is the one present in the organism, tissue and/or cell, respectively, where the expression of RTP801 is to be reduced.

“RTP801 polypeptide” refers to the polypeptide of the RTP801 gene, and is understood to include, for the purposes of the instant invention, the terms “RTP779”, “REDD1”, “DDIT4”, “FLJ20500”, “Dig2”, and “PRF1”, derived from any organism, preferably human, splice variants and fragments thereof retaining biological activity, and homologs thereof, preferably having at least 70%, more preferably at least 80%, even more preferably at least 90% or 95% homology thereto. In addition, this term is understood to encompass polypeptides resulting from minor alterations in the RTP801 coding sequence, such as, inter alia, point mutations, substitutions, deletions and insertions which may cause a difference in a few amino acids between the resultant polypeptide and the naturally occurring RTP801. RTP801 preferably has or comprises an amino acid sequence set forth in SEQ ID NO 2. It is acknowledged that there might be differences in the amino acid sequence among various tissues of an organism and among different organisms of one species or among different species to which the nucleic acid according to the present invention can be applied in various embodiments of the present invention. However, based on the technical teaching provided herein, the respective sequence can be taken into consideration accordingly when designing any of the nucleic acids according to the present invention. Particular fragments of RTP801 include amino acids 1-50, 51-100, 101-150, 151-200 and 201-232 of the sequence set forth in SEQ ID NO:2. Further particular fragments of RTP801 include amino acids 25-74, 75-124, 125-174, 175-224 and 225-232 of the sequence set forth in SEQ ID NO:2.

RTP801 as used herein is described, among others, in WO 99/09046. RTP801 has also been described as a transcriptional target of HIF-1α by Shoshani T et al. (Shoshani et al., 2002, Mol Cell Biol, 22, 2283-93). Furthermore, Ellisen et al. (Mol Cell, 2002. 10, 995-1005) has identified RTP801 as a p53-dependent DNA damage response gene and as a p63-dependent gene involved in epithelial differentiation. Also, RTP801 expression mirrors the tissue-specific pattern of the p53 family member p63, is effective similar to or in addition to TP63, and is involved in the regulation of reactive oxygen species. Apart from that, RTP801 is responsive to hypoxia-responsive transcription factor hypoxia-inducible factor 1 (HIF-1) and is typically up-regulated during hypoxia both in vitro and in vivo in an animal model of ischemic stroke. RTP801 appears to function in the regulation of reactive oxygen species (ROS). ROS levels and reduced sensitivity to oxidative stress are both increased following ectopic expression of RTP801 gene (Ellisen et al. 2002, supra; Shoshani et al. 2002, supra). Preferably, the product of RTP801 is a biologically active RTP801 protein which preferably exhibits at least one of the characteristics described hereinabove, preferable two or more and most preferably each and any of these characteristics.

The present invention relates to novel chemically modified oligonucleotides and oligoribonucleotide structures that possess therapeutic properties. In particular, the present invention discloses chemically modified siRNA compounds. The siRNAs of the present invention possess novel structures and novel modifications which have one or more of the following advantages: increased activity or reduced toxicity or reduced off-target effect or reduced immune response or increased stability; the novel modifications of the siRNAs of the present invention are beneficially applied to double stranded RNA useful in preventing or attenuation RTP801 gene expression. The siRNA compounds of the present invention comprise at least one modified nucleotide selected from the group consisting of a sugar modification, a base modification and an internucleotide linkage modification.

The present invention also relates to compounds which down-regulate expression of RTP801, particularly to novel small interfering RNAs (siRNAs), and to the use of these novel siRNAs in the treatment of various diseases and medical conditions. Particular diseases and conditions to be treated include, without being limited to, hearing loss, acute renal failure (ARF), glaucoma, diabetic retinopathy, diabetic macular edema (DME), diabetic nephropathy and other microvascular disorders, acute respiratory distress syndrome (ARDS) and other acute lung and respiratory injuries and diseases (e.g. chronic obstructive pulmonary disease (COPD)), ischemia-reperfusion injury following lung transplantation, organ transplantation including lung, liver, heart, bone marrow, pancreas, cornea and kidney transplantation, spinal cord injury, pressure sores, age-related macular degeneration (AMD), dry eye syndrome, neurodegenerative disorders, e.g. Alzheimer's disease, Parkinson's disease and ALS, oral mucositis. Other indications include chemical-induced nephrotoxicity and chemical-induced neurotoxicity, for example toxicity induced by cisplatin and cisplatin-like compounds, by aminoglycosides, by loop diuretics, and by hydroquinone and their analogs.

Lists of sense and antisense oligonucleotides useful in preparation of siRNA to be used in the present invention are provided in Tables A-I which recite SEQ ID NOS. 3-3624. 21- or 23-mer siRNA sequences can also be generated by 5′ and/or 3′ extension of the 19-mer sequences disclosed herein. Such extension is preferably complementary to the corresponding mRNA sequence.

Methods, chemically modified siRNA molecules and pharmaceutical compositions comprising these chemically modified siRNA compounds which inhibit RTP801 are discussed herein at length, and any of said molecules and/or compositions are beneficially employed in the treatment of a subject suffering from any of said conditions.

The inventors of the present invention discovered a related albeit distinct gene, RTP801L also referred to as “REDD2”. RTP801L is homologous to RTP801, and reacts in a similar manner to oxidative stress; thus, RTP801L probably possesses some similar functions with RTP801.

Without being bound by theory, RTP801 being a stress-inducible protein (responding to hypoxia, oxidative stress, thermal stress, ER stress) is a factor acting in fine-tuning of cell response to energy misbalance. As such, it is a target suitable for treatment of any disease where cells should be rescued from apoptosis due to stressful conditions (e.g. diseases accompanied by death of normal cells) or where cells, which are adapted to stressful conditions due to changes in RTP801 expression (e.g. cancer cells), should be killed. In the latter case, RTP801 is viewed as a survival factor for cancer cells and its inhibitors may treat cancer as a monotherapy or as sensitising drugs in combination with chemotherapy or radiotherapy.

By “biological effect of RTP801 in respiratory disorders” or “RTP801 biological activity in respiratory disorders” is meant the effect of RTP801 in the treatment of a subject suffering from or affected by respiratory disorders, which may be direct or indirect, and includes, without being bound by theory, the effect of RTP801 on apoptosis of alveolar cells induced by hypoxic or hyperoxic conditions. The indirect effect includes, but is not limited to, RTP801 binding to or having an effect on one of several molecules, which are involved in a signal transduction cascade resulting in apoptosis.

“Apoptosis” refers to a physiological type of cell death which results from activation of some cellular mechanisms, i.e. death that is controlled by the machinery of the cell. Apoptosis may, for example, be the result of activation of the cell machinery by an external trigger, e.g. a cytokine or anti-FAS antibody, which leads to cell death or by an internal signal. The term “programmed cell death” may also be used interchangeably with “apoptosis”.

“Apoptosis-related disease” or “apoptosis-related condition” refers to a disease whose etiology is related either wholly or partially to the process of apoptosis. The disease may be caused either by a malfunction of the apoptotic process (such as in cancer or an autoimmune disease) or by over activity of the apoptotic process (such as in certain neurodegenerative diseases). Many diseases in which RTP801 is involved are apoptosis-related diseases. For example, apoptosis is a significant mechanism in dry AMD, whereby slow atrophy of photoreceptor and pigment epithelium cells, primarily in the central (macular) region of retina takes place. Neuroretinal apoptosis is also a significant mechanism in diabetic retinopathy.

“Angiogenesis” refers to the process by which living cells, tissues, or organisms form new blood vessels. Angiogenesis is a fundamental biological process which plays a central role in the pathogenesis of various conditions, and is a major contributor to mortality and morbidity in diseases, such as cancer, diabetic retinopathy, and macular degeneration (Folkman, 1990, JNCI 82: 4-6).

“Angiogenesis-related condition” refers to any one of the medical conditions or disease states recognized to be influenced by angiogenesis or by an increase/decrease in angiogenesis of by the lack thereof, including conditions which may be linked to angiogenesis in the future. Examples of such conditions include cancer, retinopathy, ischemia, macular degeneration, corneal diseases, glaucoma, diabetic retinopathy, stroke, ischemic heart disease, ulcers, scleradoma, myocardial infarction, myocardial angiogenesis, plaque neovascularization, ischemic limb angiogenesis, angina pectoris, unstable angina, coronary arteriosclerosis, arteriosclerosis obliterans, Berger's disease, arterial embolism, arterial thrombosis, cerebrovascular occlusion, cerebral infarction, cerebral thrombosis, cerebral embolism, inflammation, diabetic neovascularization, wound healing and peptic ulcer.

An “RTP801 inhibitor” is a siRNA compound which is capable of inhibiting the activity of the RTP801 gene or RTP801 gene product, particularly the human RTP801 gene or gene product. Such inhibitors affect the transcription or translation of the RTP801 gene. In some embodiments an RTP801 inhibitor is a siRNA inhibitor of the RTP801 promoter. Specific chemically modified siRNA inhibitors of RTP801 gene are provided hereinbelow.

Hypoxia has been recognised as a key element in the pathomechanism of quite a number of diseases such as stroke, emphysema and infarct, which are associated with sub-optimum oxygen availability and tissue damaging responses to the hypoxia conditions. In fast-growing tissues, including tumor, sub-optimum oxygen availability is compensated by undesired neo-angiogenesis. Therefore, at least in case of cancer diseases, the growth of vasculature is undesired.

Another objective of the present invention is thus to provide compositions and methods for the treatment of diseases involving undesired growth of vasculature and angiogenesis, respectively.

The present invention provides methods and compositions for inhibiting expression of RTP801 gene in vivo. In general, the method includes administering oligoribonucleotides, in particular small interfering RNAs (i.e. siRNAs) that target an mRNA transcribed from the RTP801 gene in an amount sufficient to down-regulate expression of the RTP801 gene by an RNA interference mechanism. In particular, the subject method can be used to inhibit expression of the RTP801 gene for treatment of a disease. In accordance with the present invention, the siRNA compounds of the invention are used as drugs to treat various pathologies. In particular, the subject method can be used to inhibit expression for treatment of a disease or a disorder or a condition disclosed herein.

The present invention provides chemically modified siRNA compounds, which down-regulate the expression of a RTP801 gene transcribed into mRNA having a polynucleotide sequence set forth in SEQ ID NO:1 and pharmaceutical compositions comprising one or more such siRNA compounds.

A siRNA of the invention is a duplex oligoribonucleotide in which the sense strand is substantially complementary to an 18-40 consecutive nucleotide segment of the mRNA polynucleotide sequence of RTP801 gene, and the antisense strand is substantially complementary to the sense strand. In general, some deviation from the target mRNA sequence is tolerated without compromising the siRNA activity (see e.g. Czauderna et al., Nuc. Acids Res. 2003, 31(10:2705-2716). A siRNA of the invention inhibits RTP801 gene expression on a post-transcriptional level with or without destroying the mRNA. Without being bound by theory, siRNA targets the mRNA for specific cleavage and degradation and/or inhibits translation from the targeted message.

In some embodiments the siRNA is blunt ended, on one or both ends. More specifically, in some embodiments the siRNA is blunt ended on the end defined by the 5′-terminus of the first strand and the 3′-terminus of the second strand, or the end defined by the 3′-terminus of the first strand and the 5′-terminus of the second strand.

In other embodiments at least one of the two strands has an overhang of at least one nucleotide at the 5′-terminus; the overhang comprises at least one deoxyribonucleotide. At least one of the strands also optionally has an overhang of at least one nucleotide at the 3′-terminus. The overhang consists of from about 1 to about 5 nucleotides.

The length of RNA duplex is from about 18 to about 40 ribonucleotides, preferably 19 to 23 ribonucleotides. In some embodiments the length of each strand (oligomer) is independently selected from the group consisting of about 18 to about 40 bases, preferably 18 to 23 bases and more preferably 19, 20 or 21 ribonucleotides.

Additionally, in certain preferred embodiments the complementarity between said first strand and the target nucleic acid is perfect. In some embodiments, the strands are substantially complementary, i.e. having one, two or up to three mismatches between said first strand and the target nucleic acid.

In some embodiments the 5′-terminus of the first strand of the siRNA is linked to the 3′-terminus of the second strand, or the 3′-terminus of the first strand is linked to the 5′-terminus of the second strand, said linkage being via a nucleic acid linker typically having a length between 3-100 nucleotides, preferably about 3 to about 10 nucleotides.

The siRNAs compounds of the present invention possess structures and modifications which impart one or more of increased activity, increased stability, reduced toxicity, reduced off target effect, and/or reduced immune response. The siRNA structures of the present invention are beneficially applied to double stranded RNA useful in preventing or attenuating expression of RTP801 gene.

The present invention also relates to the use of the chemically modified siRNAs in the treatment of various diseases and medical conditions. Particular diseases and conditions to be treated are ARDS; COPD; ALI; Emphysema; Diabetic Neuropathy, nephropathy and retinopathy; DME and other diabetic conditions; Glaucoma; AMD; BMT retinopathy; ischemic conditions including stroke; OIS; Neurodegenerative disorders such as Parkinson's, Alzheimer's, ALS; kidney disorders: ARF, DGF, transplant rejection; hearing disorders; spinal cord injuries; oral mucositis; dry eye syndrome and pressure sores. Lists of siRNA to be used in the present invention are provided in Tables A-I. Tables A, B, D, E and I set forth 19-mer oligomers. Tables C and F set forth 21-mer oligomers. Tables G and H set forth 23-mer oligomers. 21- or 23-mer siRNA sequences can also be generated by 5′ and/or 3′ extension of the 19-mer sequences disclosed herein. Such extension is preferably complementary to the corresponding mRNA sequence.

Tables A-C include oligonucleotide pairs set forth in SEQ ID NOS:3-344, were disclosed by the assignee of the present invention in PCT Patent Application No. PCT/US2005/029236, published as WO 2006/023544. Table D includes oligonucleotide pairs set forth in SEQ ID NOS:345-412, were disclosed by the assignee of the present invention in PCT Patent Application No. PCT/US2008/002483, published as WO 2008/106102.

Methods, molecules and compositions of the present invention which inhibit the RTP801 gene are discussed herein at length, and any of said molecules and/or compositions are beneficially employed in the treatment of a subject suffering from one or more of said conditions.

Where aspects or embodiments of the invention are described in terms of Markush groups or other grouping of alternatives, those skilled in the art will recognize that the invention is also thereby described in terms of any individual member or subgroup of members of the group.

siRNA Oligonucleotides

Tables A-I provide nucleic acid sequences of sense and corresponding antisense oligonucleotides, useful in preparing chemically modified siRNA compounds of the invention. Antisense and corresponding sense oligonucleotides useful in preparing siRNA according to the present invention are set forth in SEQ ID NOS:3-3624. Throughout the specification, nucleotide positions are numbered from 1 to 19 or 1 to 21 or 1 to 23 and are counted from the 5′ end of the antisense or sense oligonucleotides. For example, position 1 on (N)x refers to the 5′ terminal nucleotide on the antisense oligonucleotide strand and position 1 on (N′)y refers to the 5′ terminal nucleotide on the sense oligonucleotide strand.

According to the present invention the siRNA compounds are chemically and or structurally modified according to one of the following modifications set forth in Structures (A)-(P) or as tandem siRNA or RNAstar.

In one aspect the present invention provides a compound set forth as Structure (A):

| | | |
|---|---|---|
| (A) | 5′ (N)ₓ-Z 3′ | (antisense strand) |
| | 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present;
wherein the sequence of (N′)_{y} is a sequence substantially complementary to (N)x; and
wherein the sequence of (N)ₓ comprises an antisense sequence substantially identical to an antisense sequence disclosed in any one of Tables E-I.

In certain embodiments the present invention provides a compound having structure (B):

| | | |
|---|---|---|
| (B) | 5′ (N)x-Z 3′ | antisense strand |
| | 3′ Z-′(N′)y 5′ | sense strand |

wherein each of (N)ₓ and (N′)_{y} is an oligomer in which each consecutive N or N′ is an unmodified ribonucleotide or a modified ribonucleotide joined to the next N or N′ by a covalent bond;
wherein each of x and y=19, 21 or 23 and (N)ₓ and (N′)_{y} are fully complementary
wherein each of Z and Z′ may be present or absent, but if present is 1-5 consecutive nucleotides covalently attached at the 3′ terminus of the strand in which it is present;
wherein alternating ribonucleotides in each of (N)ₓ and (N′)_{y} are 2′-O-methyl sugar modified ribonucleotides;
wherein the sequence of (N′)_{y} is a sequence substantially complementary to (N)x; and
wherein the sequence of (N)ₓ comprises an antisense sequence substantially identical to an antisense sequence disclosed in any one of Tables E-I

In some embodiments each of (N)ₓ and (N′)_{y} is independently phosphorylated or non-phosphorylated at the 3′ and 5′ termini.

In certain embodiments wherein each of x and y=19 or 23, each N at the 5′ and 3′ termini of (N)ₓ is modified; and each N′ at the 5′ and 3′ termini of (N′)_{y} is unmodified.

In certain embodiments wherein each of x and y=21, each N at the 5′ and 3′ termini of (N)ₓ is unmodified; and each N′ at the 5′ and 3′ termini of (N′)_{y} is modified.

In particular embodiments, x and y=19, and the siRNA is modified such that a 2′-O-methyl sugar modified ribonucleotide (2′-OMe) is present in the first, third, fifth, seventh, ninth, eleventh, thirteenth, fifteenth, seventeenth and nineteenth positions of the antisense strand (N)ₓ, and a 2′-OMe sugar modified ribonucleotide is present in the second, fourth, sixth, eighth, tenth, twelfth, fourteenth, sixteenth and eighteenth positions of the sense strand (N′)_{y}.

In some embodiments, the present invention provides a compound having Structure (C):

| | | |
|---|---|---|
| (C) | 5′ (N)x-Z 3′ | antisense strand |
| | 3′ Z′-(N′)y 5′ | sense strand |

wherein each of N and N′ is a nucleotide independently selected from an unmodified ribonucleotide, a modified ribonucleotide, an unmodified deoxyribonucleotide and a modified deoxyribonucleotide;
wherein each of (N)x and (N′)y is an oligomer in which each consecutive nucleotide is joined to the next nucleotide by a covalent bond and each of x and y is an integer between 18 and 40;
wherein in (N)x the nucleotides are unmodified or (N)x comprises alternating modified ribonucleotides and unmodified ribonucleotides; each modified ribonucleotide being modified so as to have a 2′-O-methyl on its sugar and the ribonucleotide located at the middle position of (N)x being modified or unmodified preferably unmodified;
wherein (N′)y comprises unmodified ribonucleotides further comprising one modified nucleotide at a terminal or penultimate position, wherein the modified nucleotide is selected from the group consisting of a mirror nucleotide, a bicyclic nucleotide, a 2′-sugar modified nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein if more than one nucleotide is modified in (N′)y, the modified nucleotides may be consecutive;
wherein each of Z and Z′ may be present or absent, but if present is 1-5 deoxyribonucleotides covalently attached at the 3′ terminus of any oligomer to which it is attached;
wherein the sequence of (N′)_{y} comprises a sequence substantially complementary to (N)x; and wherein the sequence of (N)ₓ comprises an antisense sequence substantially complementary to about 18 to about 40 consecutive ribonucleotides in mRNA of the RTP801 gene, set forth in SEQ ID NO: 1. Preferably (N)ₓ comprises an antisense sequence substantially identical to an antisense sequence set forth in any one of Tables A-I.

In particular embodiments, x=y=19 and in (N)x each modified ribonucleotide is modified so as to have a 2′-O-methyl on its sugar and the ribonucleotide located at the middle of (N)x is unmodified. Accordingly, in a compound wherein x=˜19, (N)x comprises 2′-O-methyl sugar modified ribonucleotides at positions 1, 3, 5, 7, 9, 11, 13, 15, 17 and 19. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 5. In other embodiments, (N)x comprises 2′O Me modified ribonucleotides at positions 2, 4, 8, 11, 13, 15, 17 and 19 and may further comprise at least one abasic or inverted abasic unconventional moiety for example in position 6. In other embodiments, (N)x comprises 2′O Me modified ribonucleotides at positions 2, 4, 6, 8, 11, 13, 17 and 19 and may further comprise at least one abasic or inverted abasic unconventional moiety for example in position 15. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 14. In other embodiments, (N)x comprises 2′O Me modified ribonucleotides at positions 1, 2, 3, 7, 9, 11, 13, 15, 17 and 19 and may further comprise at least one abasic or inverted abasic unconventional moiety for example in position 5. In other embodiments, (N)x comprises 2′O Me modified ribonucleotides at positions 1, 2, 3, 5, 7, 9, 11, 13, 15, 17 and 19 and may further comprise at least one abasic or inverted abasic unconventional moiety for example in position 6. In other embodiments, (N)x comprises 2′O Me modified ribonucleotides at positions 1, 2, 3, 5, 7, 9, 11, 13, 17 and 19 and may further comprise at least one abasic or inverted abasic unconventional moiety for example in position 15. In other embodiments, (N)x comprises 2′O Me modified ribonucleotides at positions 1, 2, 3, 5, 7, 9, 11, 13, 15, 17 and 19 and may further comprise at least one abasic or inverted abasic unconventional moiety for example in position 14. In other embodiments, (N)x comprises 2′O Me modified ribonucleotides at positions 2, 4, 6, 7, 9, 11, 13, 15, 17 and 19 and may further comprise at least one abasic or inverted abasic unconventional moiety for example in position 5. In other embodiments, (N)x comprises 2′O Me modified ribonucleotides at positions 1, 2, 4, 6, 7, 9, 11, 13, 15, 17 and 19 and may further comprise at least one abasic or inverted abasic unconventional moiety for example in position 5. In other embodiments, (N)x comprises 2′O Me modified ribonucleotides at positions 2, 4, 6, 8, 11, 13, 14, 16, 17 and 19 and may further comprise at least one abasic or inverted abasic unconventional moiety for example in position 15. In other embodiments, (N)x comprises 2′O Me modified ribonucleotides at positions 1, 2, 3, 5, 7, 9, 11, 13, 14, 16, 17 and 19 and may further comprise at least one abasic or inverted abasic unconventional moiety for example in position 15. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 7. In other embodiments, (N)x comprises 2′O-Me modified ribonucleotides at positions 2, 4, 6, 11, 13, 15, 17 and 19 and may further comprise at least one abasic or inverted abasic unconventional moiety for example in position 8. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 9. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 10. In other embodiments, (N)x comprises 2′O Me modified ribonucleotides at positions 2, 4, 6, 8, 13, 15, 17 and 19 and may further comprise at least one abasic or inverted abasic unconventional moiety for example in position 11. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 12. In other embodiments, (N)x comprises 2′O-Me modified ribonucleotides at positions 2, 4, 6, 8, 11, 15, 17 and 19 and may further comprise at least one abasic or inverted abasic unconventional moiety for example in position 13.

In yet other embodiments (N)x comprises at least one nucleotide mismatch relative to the RTP801 mRNA. In certain preferred embodiments, (N)x comprises a single nucleotide mismatch on position 5, 6, or 14. In one embodiment of Structure (C), at least two nucleotides at either or both the 5′ and 3′ termini of (N′)y are joined by a 2′-5′ phosphodiester bond. In certain preferred embodiments x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and three nucleotides at the 3′ terminus of (N′)y are joined by two 2′-5′ phosphodiester bonds (set forth herein as Structure I). In other preferred embodiments, x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and four consecutive nucleotides at the 5′ terminus of (N′)y are joined by three 2′-5′ phosphodiester bonds. In a further embodiment, an additional nucleotide located in the middle position of (N)y may be modified with 2′-O-methyl on its sugar. In another preferred embodiment, in (N)x the nucleotides alternate between 2′-O-methyl modified ribonucleotides and unmodified ribonucleotides, and in (N′)y four consecutive nucleotides at the 5′ terminus are joined by three 2′-5′ phosphodiester bonds and the 5′ terminal nucleotide or two or three consecutive nucleotides at the 5′ terminus comprise 3′-O-methyl modifications.

In certain preferred embodiments of Structure C, x=y=19 and in (N′)y, at least one position comprises an abasic or inverted abasic unconventional moiety, preferably five positions comprises an abasic or inverted abasic unconventional moieties. In various embodiments, the following positions comprise an abasic or inverted abasic: positions 1 and 16-19, positions 15-19, positions 1-2 and 17-19, positions 1-3 and 18-19, positions 1-4 and 19 and positions 1-5. (N′)y may further comprise at least one LNA nucleotide.

In certain preferred embodiments of Structure C, x=y=19 and in (N′)y the nucleotide in at least one position comprises a mirror nucleotide, a deoxyribonucleotide and a nucleotide joined to an adjacent nucleotide by a 2′-5′ internucleotide bond.

In certain preferred embodiments of Structure C, x=y=19 and (N′)y comprises a mirror nucleotide. In various embodiments the mirror nucleotide is an L-DNA nucleotide. In certain embodiments the L-DNA is L-deoxyribocytidine. In some embodiments (N′)y comprises L-DNA at position 18. In other embodiments (N′)y comprises L-DNA at positions 17 and 18. In certain embodiments (N′)y comprises L-DNA substitutions at positions 2 and at one or both of positions 17 and 18. In certain embodiments (N′)y further comprises a 5′ terminal cap nucleotide such as 5′-O-methyl DNA or an abasic or inverted abasic moiety as an overhang.

In yet other embodiments (N′)y comprises a DNA at position 15 and L-DNA at one or both of positions 17 and 18. In that structure, position 2 may further comprise an L-DNA or an abasic unconventional moiety.

Other embodiments of Structure C are envisaged wherein x=y=21 in these embodiments the modifications for (N′)y discussed above instead of being on positions 15, 16, 17, 18 are on positions 17, 18, 19, 20 for 21-mer; similarly the modifications at one or both of positions 17 and 18 are on one or both of positions 19 or 20 for the 21-mer. All modifications in the 19-mer are similarly adjusted for the 21- and 23-mer.

According to various embodiments of Structure (C), in (N′)y 2, 3, 4, 5, 6, 7, 8, 9, 10, 11, 12, 13 or 14 consecutive ribonucleotides at the 3′ terminus are linked by 2′-5′ internucleotide linkages. In one preferred embodiment, four consecutive nucleotides at the 3′ terminus of (N′)y are joined by three 2′-5′ phosphodiester bonds, wherein one or more of the 2′-5′ nucleotides which form the 2′-5′ phosphodiester bonds further comprises a 3′-O-methyl sugar modification. Preferably the 3′ terminal nucleotide of (N′)y comprises a 2′-O-methyl sugar modification. In certain preferred embodiments of Structure C, x=y=19 and in (N′)y two or more consecutive nucleotides at positions 15, 16, 17, 18 and 19 comprise a nucleotide joined to an adjacent nucleotide by a 2′-5′ internucleotide bond. In various embodiments the nucleotide forming the 2′-5′ internucleotide bond comprises a 3′ deoxyribose nucleotide or a 3′ methoxy nucleotide. In some embodiments the nucleotides at positions 17 and 18 in (N′)y are joined by a 2′-5′ internucleotide bond. In other embodiments the nucleotides at positions 16-17, 17-18, or 16-18 in (N′)y are joined by a 2′-5′ internucleotide bond.

In certain embodiments (N′)y comprises an L-DNA at position 2 and 2′-5′ internucleotide bonds at positions 16-17, 17-18, or 16-18. In certain embodiments (N′)y comprises 2′-5′ internucleotide bonds at positions 16-17, 17-18, or 16-18 and a 5′ terminal cap nucleotide.

According to various embodiments of Structure (C), in (N′)y 2, 3, 4, 5, 6, 7, 8, 9, 10, 11, 12, 13 or 14 consecutive nucleotides at either terminus or 2-8 modified nucleotides at each of the 5′ and 3′ termini are independently mirror nucleotides. In some embodiments the mirror nucleotide is an L-ribonucleotide. In other embodiments the mirror nucleotide is an L-deoxyribonucleotide. The mirror nucleotide may further be modified at the sugar or base moiety or in an internucleotide linkage.

In one preferred embodiment of Structure (C), the 3′ terminal nucleotide or two or three consecutive nucleotides at the 3′ terminus of (N′)y are L-deoxyribonucleotides.

In other embodiments of Structure (C), in (N′)y 2, 3, 4, 5, 6, 7, 8, 9, 10, 11, 12, 13 or 14 consecutive ribonucleotides at either terminus or 2-8 modified nucleotides at each of the 5′ and 3′ termini are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe). In one series of preferred embodiments, three, four or five consecutive nucleotides at the 5′ terminus of (N′)y comprise the 2′-OMe modification. In another preferred embodiment, three consecutive nucleotides at the 3′ terminus of (N′)y comprise the 2′-O-methyl modification.

In some embodiments of Structure (C), in (N′)y 2, 3, 4, 5, 6, 7, 8, 9, 10, 11, 12, 13 or 14 consecutive ribonucleotides at either or 2-8 modified nucleotides at each of the 5′ and 3′ termini are independently bicyclic nucleotides. In various embodiments the bicyclic nucleotide is a locked nucleic acid (LNA) or a species of LNA, e.g. 2′-O, 4′-C-ethylene-bridged nucleic acid (ENA) is a species of LNA.

In various embodiments (N′)y comprises modified nucleotides at the 5′ terminus or at both the 3′ and 5′ termini.

In some embodiments of Structure (C), at least two nucleotides at either or both the 5′ and 3′ termini of (N′)y are joined by P-ethoxy backbone modifications. In certain preferred embodiments x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle position of (N)x being unmodified; and four consecutive nucleotides at the 3′ terminus or at the 5′ terminus of (N′)y are joined by three P-ethoxy backbone modifications. In another preferred embodiment, three consecutive nucleotides at the 3′ terminus or at the 5′ terminus of (N′)y are joined by two P-ethoxy backbone modifications.

In some embodiments of Structure (C), in (N′)y 2, 3, 4, 5, 6, 7 or 8, consecutive ribonucleotides at each of the 5′ and 3′ termini are independently mirror nucleotides, nucleotides joined by 2′-5′ phosphodiester bond, 2′ sugar modified nucleotides or bicyclic nucleotide. In one embodiment, the modification at the 5′ and 3′ termini of (N′)y is identical. In one preferred embodiment, four consecutive nucleotides at the 5′ terminus of (N′)y are joined by three 2′-5′ phosphodiester bonds and three consecutive nucleotides at the 3′ terminus of (N′)y are joined by two 2′-5′ phosphodiester bonds. In another embodiment, the modification at the 5′ terminus of (N′)y is different from the modification at the 3′ terminus of (N′)y. In one specific embodiment, the modified nucleotides at the 5′ terminus of (N′)y are mirror nucleotides and the modified nucleotides at the 3′ terminus of (N′)y are joined by 2′-5′ phosphodiester bond. In another specific embodiment, three consecutive nucleotides at the 5′ terminus of (N′)y are LNA nucleotides and three consecutive nucleotides at the 3′ terminus of (N′)y are joined by two 2′-5′ phosphodiester bonds. In (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified, or the ribonucleotides in (N)x being unmodified.

In another embodiment of Structure (C), the present invention provides a compound wherein x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; three nucleotides at the 3′ terminus of (N′)y are joined by two 2′-5′ phosphodiester bonds and three nucleotides at the 5′ terminus of (N′)y are LNA such as ENA.

In another embodiment of Structure (C), five consecutive nucleotides at the 5′ terminus of (N′)y comprise the 2′-O-methyl sugar modification and two consecutive nucleotides at the 3′ terminus of (N′)y are L-DNA.

In yet another embodiment, the present invention provides a compound wherein x=y=19 or x=y=23; (N)x consists of unmodified ribonucleotides; three consecutive nucleotides at the 3′ terminus of (N′)y are joined by two 2′-5′ phosphodiester bonds and three consecutive nucleotides at the 5′ terminus of (N′)y are LNA such as ENA.

According to other embodiments of Structure (C), in (N′)y the 5′ or 3′ terminal nucleotide, or 2, 3, 4, 5 or 6 consecutive nucleotides at either termini or 1-4 modified nucleotides at each of the 5′ and 3′ termini are independently phosphonocarboxylate or phosphinocarboxylate nucleotides (PACE nucleotides). In some embodiments the PACE nucleotides are deoxyribonucleotides. In some preferred embodiments in (N′)y, 1 or 2 consecutive nucleotides at each of the 5′ and 3′ termini are PACE nucleotides.

In additional embodiments, the present invention provides a compound having Structure (D):

| | | |
|---|---|---|
| (D) | 5′ (N)x-Z 3′ | antisense strand |
| | 3′ Z′-(N′)y 5′ | sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is independently an integer between 18 and 40;
wherein (N)x comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and wherein the sequence of (N)ₓ comprises an antisense sequence substantially complementary to about 18 to about 40 consecutive ribonucleotides in a RTP801 mRNA. Preferably (N)ₓ comprises an antisense sequence substantially identical to an antisense sequence set forth in any one of Tables A-I.

In one embodiment of Structure (D), x=y=19 or x=y=23; (N)x comprises unmodified ribonucleotides in which two consecutive nucleotides linked by one 2′-5′ internucleotide linkage at the 3′ terminus; and (N′)y comprises unmodified ribonucleotides in which two consecutive nucleotides are linked by one 2′-5′ internucleotide linkage at the 5′ terminus.

In some embodiments, x=y=19 or x=y=23; (N)x comprises unmodified ribonucleotides in which three consecutive nucleotides at the 3′ terminus are joined together by two 2′-5′ phosphodiester bonds; and (N′)y comprises unmodified ribonucleotides in which four consecutive nucleotides at the 5′ terminus are joined together by three 2′-5′ phosphodiester bonds (set forth herein as Structure II).

According to various embodiments of Structure (D), 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are linked by 2′-5′ internucleotide linkages.

According to one preferred embodiment of Structure (D), four consecutive nucleotides at the 5′ terminus of (N′)y are joined by three 2′-5′ phosphodiester bonds and three consecutive nucleotides at the 3′ terminus of (N′)x are joined by two 2′-5′ phosphodiester bonds. Three nucleotides at the 5′ terminus of (N′)y and two nucleotides at the 3′ terminus of (N′)x may also comprise 3′-O-methyl modifications.

According to various embodiments of Structure (D), 2, 3, 4, 5, 6, 7, 8, 9, 10, 11, 12, 13 or 14 consecutive nucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently mirror nucleotides. In some embodiments the mirror is an L-ribonucleotide. In other embodiments the mirror nucleotide is L-deoxyribonucleotide.

In other embodiments of Structure (D), 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently 2′ sugar modified nucleotides. In some embodiments the 2′ sugar modification comprises an amino, a fluoro, an alkoxy or an alkyl moiety. In certain embodiments the 2′ sugar modification comprises a methoxy moiety (2′-OMe).

In one preferred embodiment of Structure (D), five consecutive nucleotides at the 5′ terminus of (N′)y comprise the 2′-O-methyl modification and five consecutive nucleotides at the 3′ terminus of (N′)x comprise the 2′-O-methyl modification. In another preferred embodiment of Structure (D), ten consecutive nucleotides at the 5′ terminus of (N′)y comprise the 2′-O-methyl modification and five consecutive nucleotides at the 3′ terminus of (N′)x comprise the 2′-O-methyl modification. In another preferred embodiment of Structure (D), thirteen consecutive nucleotides at the 5′ terminus of (N′)y comprise the 2′-O-methyl modification and five consecutive nucleotides at the 3′ terminus of (N′)x comprise the 2′-O-methyl modification.

In some embodiments of Structure (D), 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (D), (N′)y comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In various embodiments of Structure (D), (N)x comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In some embodiments wherein each of the 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In one specific embodiment of Structure (D), five consecutive nucleotides at the 5′ terminus of (N′)y comprise the 2′-O-methyl modification and two consecutive nucleotides at the 3′ terminus of (N′)y are L-DNA. In addition, the compound may further comprise five consecutive 2′-O-methyl modified nucleotides at the 3′ terminus of (N′)x.

In various embodiments of Structure (D), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (E):

| | | |
|---|---|---|
| (E) | 5′ (N)x-Z 3′ | antisense strand |
| | 3′ Z′-(N′)y 5′ | sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is independently an integer between 18 and 40;
wherein (N)x comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and wherein the sequence of (N)ₓ comprises an antisense sequence substantially complementary to about 18 to about 40 consecutive ribonucleotides in a RTP801 mRNA. Preferably (N)ₓ comprises an antisense sequence substantially identical to an antisense sequence set forth in any one of Tables A-I.

In certain preferred embodiments the ultimate nucleotide at the 5′ terminus of (N)x is unmodified.

According to various embodiments of Structure (E), 2, 3, 4, 5, 6, 7, 8, 9, 10, 11, 12, 13 or 14 consecutive ribonucleotides starting at the ultimate or penultimate position of the 5′ terminus of (N)ₓ, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are linked by 2′-5′ internucleotide linkages.

According to various embodiments of Structure (E), 2, 3, 4, 5, 6, 7, 8, 9, 10, 11, 12, 13 or 14 consecutive nucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive nucleotides starting at the ultimate or penultimate position of the 3′ terminus of (N′)y are independently mirror nucleotides. In some embodiments the mirror is an L-ribonucleotide. In other embodiments the mirror nucleotide is L-deoxyribonucleotide.

In other embodiments of Structure (E), 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently 2′ sugar modified nucleotides. In some embodiments the 2′ sugar modification comprises an amino, a fluoro, an alkoxy or an alkyl moiety. In certain embodiments the 2′ sugar modification comprises a methoxy moiety (2′-OMe).

In some embodiments of Structure (E), 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (E), (N′)y comprises modified nucleotides selected from a bicyclic nucleotide, a 2′ sugar modified nucleotide, a mirror nucleotide, an altritol nucleotide, a nucleotide joined to an adjacent nucleotide by an internucleotide linkage selected from a 2′-5′ phosphodiester bond, a P-alkoxy linkage or a PACE linkage at the 3′ terminus or at each of the 3′ and 5′ termini.

In various embodiments of Structure (E), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where both 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (E), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (F):

| | | |
|---|---|---|
| (F) | 5′ (N)x-Z 3′ | antisense strand |
| | 3′ Z′-(N′)y 5′ | sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is independently an integer between 18 and 40;
wherein each of (N)x and (N′)y comprise unmodified ribonucleotides in which each of (N)x and (N′)y independently comprise one modified nucleotide at the 3′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a PACE linkage or a nucleotide joined to an adjacent nucleotide by a 2′-5′ phosphodiester bond;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)y is a sequence substantially complementary to (N)x; and wherein the sequence of (N)ₓ comprises an antisense sequence substantially complementary to about 18 to about 40 consecutive ribonucleotides in a RTP801 mRNA. Preferably (N)ₓ comprises an antisense sequence substantially identical to an antisense sequence set forth in any one of Tables A-I.

In some embodiments of Structure (F), x=y=19 or x=y=23; (N′)y comprises unmodified ribonucleotides in which two consecutive nucleotides at the 3′ terminus comprise two consecutive mirror deoxyribonucleotides; and (N)x comprises unmodified ribonucleotides in which one nucleotide at the 3′ terminus comprises a mirror deoxyribonucleotide (set forth as Structure III).

According to various embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are linked by 2′-5′ internucleotide linkages.

According to one preferred embodiment of Structure (F), three consecutive nucleotides at the 3′ terminus of (N′)y are joined by two 2′-5′ phosphodiester bonds and three consecutive nucleotides at the 3′ terminus of (N′)x are joined by two 2′-5′ phosphodiester bonds.

According to various embodiments of Structure (F), 2, 3, 4, 5, 6, 7, 8, 9, 10, 11, 12, 13 or 14 consecutive nucleotides independently beginning at the ultimate or penultimate position of the 3′ termini of (N)x and (N′)y are independently mirror nucleotides. In some embodiments the mirror nucleotide is an L-ribonucleotide. In other embodiments the minor nucleotide is an L-deoxyribonucleotide.

In other embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently 2′ sugar modified nucleotides. In some embodiments the 2′ sugar modification comprises an amino, a fluoro, an alkoxy or an alkyl moiety. In certain embodiments the 2′ sugar modification comprises a methoxy moiety (2′-OMe).

In some embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (F), (N′)y comprises a modified nucleotide selected from a bicyclic nucleotide, a 2′ sugar modified nucleotide, a minor nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at both the 3′ and 5′ termini.

In various embodiments of Structure (F), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (F), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (G)

| | | |
|---|---|---|
| (G) | 5′ (N)x-Z 3′ | antisense strand |
| | 3′ Z′-(N′)y 5′ | sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is independently an integer between 18 and 40;
wherein each of (N)x and (N′)y comprise unmodified ribonucleotides in which each of (N)x and (N′)y independently comprise one modified nucleotide at the 5′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a PACE linkage or a nucleotide joined to an adjacent nucleotide by a 2′-5′ phosphodiester bond;
wherein for (N)x the modified nucleotide is preferably at penultimate position of the 5′ terminal;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and wherein the sequence of (N)ₓ comprises an antisense sequence substantially complementary to about 18 to about 40 consecutive ribonucleotides in a RTP801 mRNA. Preferably (N)ₓ comprises an antisense sequence substantially identical to an antisense sequence set forth in any one of Tables A-I.

In some embodiments of Structure (G), x=y=19 or x=y=23.

According to various embodiments of Structure (G), 2, 3, 4, 5, 6, 7, 8, 9, 10, 11, 12, 13 or 14 consecutive ribonucleotides independently beginning at the ultimate or penultimate position of the 5′ termini of (N)x and (N′)y are linked by 2′-5′ internucleotide linkages. For (N)x the modified nucleotides preferably start at the penultimate position of the 5′ terminal.

According to various embodiments of Structure (G), 2, 3, 4, 5, 6, 7, 8, 9, 10, 11, 12, 13 or 14 consecutive nucleotides independently beginning at the ultimate or penultimate position of the 5′ termini of (N)x and (N′)y are independently mirror nucleotides. In some embodiments the mirror nucleotide is an L-ribonucleotide. In other embodiments the mirror nucleotide is an L-deoxyribonucleotide. For (N)x the modified nucleotides preferably start at the penultimate position of the 5′ terminal.

In other embodiments of Structure (G), 2, 3, 4, 5, 6, 7, 8; 9, 10, 11, 12, 13 or 14 consecutive ribonucleotides independently beginning at the ultimate or penultimate position of the 5′ termini of (N)x and (N′)y are independently 2′ sugar modified nucleotides. In some embodiments the 2′ sugar modification comprises an amino, a fluoro, an alkoxy or an alkyl moiety. In certain embodiments the 2′ sugar modification comprises a methoxy moiety (2′-OMe). In some preferred embodiments the consecutive modified nucleotides preferably begin at the penultimate position of the 5′ terminus of (N)x.

In one preferred embodiment of Structure (G), five consecutive ribonucleotides at the 5′ terminus of (N′)y comprise a 2′-O-methyl modification and one ribonucleotide at the 5′ penultimate position of (N′)x comprises a 2′-O-methyl modification. In another preferred embodiment of Structure (G), five consecutive ribonucleotides at the 5′ terminus of (N′)y comprise a 2′-O-methyl modification and two consecutive ribonucleotides at the 5′ terminal position of (N′)x comprise a 2′-O-methyl modification.

In some embodiments of Structure (G), 2, 3, 4, 5, 6, 7, 8, 9, 10, 11, 12, 13 or 14 consecutive ribonucleotides independently beginning at the ultimate or penultimate position of the 5′ termini of (N)x and (N′)y are bicyclic nucleotides. In various embodiments the bicyclic nucleotide is a locked nucleic acid (LNA) such as a 2′-O, 4′-C-ethylene-bridged nucleic acid (ENA). In some preferred embodiments the consecutive modified nucleotides preferably begin at the penultimate position of the 5′ terminus of (N)x.

In various embodiments of Structure (G), (N′)y comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In various embodiments of Structure (G), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond. In various embodiments of Structure (G), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are minor nucleotides.

In additional embodiments, the present invention provides a compound having Structure (H):

| | | |
|---|---|---|
| (H) | 5′ (N)x-Z 3′ | antisense strand |
| | 3′ Z′-(N′)y 5′ | sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is independently an integer between 18 and 40;
wherein (N)x comprises unmodified ribonucleotides further comprising one modified nucleotide at the 3′ terminal or penultimate position or the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at an internal position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)y is a sequence substantially complementary to (N)x; and wherein the sequence of (N)x comprises an antisense sequence substantially complementary to about 18 to about 40 consecutive ribonucleotides in a RTP801 mRNA. Preferably (N)x comprises an antisense sequence substantially identical to an antisense sequence set forth in any one of Tables A-I.

In one embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or both termini of (N)x are independently 2′ sugar modified nucleotides, bicyclic nucleotides, mirror nucleotides, altritol nucleotides or nucleotides joined to an adjacent nucleotide by a 2′-5′ phosphodiester bond and 2, 3, 4, 5, 6, 7, 8, 9, 10, 11, 12, 13 or 14 consecutive internal ribonucleotides in (N′)y are independently 2′ sugar modified nucleotides, bicyclic nucleotides, mirror nucleotides, altritol nucleotides or nucleotides joined to an adjacent nucleotide by a 2′-5′ phosphodiester bond. In some embodiments the 2′ sugar modification comprises an amino, a fluoro, an alkoxy or an alkyl moiety. In certain embodiments the 2′ sugar modification comprises a methoxy moiety (2′-OMe).

In another embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or 2-8 consecutive nucleotides at each of 5′ and 3′ termini of (N′)y are independently 2′ sugar modified nucleotides, bicyclic nucleotides, mirror nucleotides, altritol nucleotides or nucleotides joined to an adjacent nucleotide by a 2′-5′ phosphodiester bond, and 2, 3, 4, 5, 6, 7, 8, 9, 10, 11, 12, 13 or 14 consecutive internal ribonucleotides in (N)x are independently 2′ sugar modified nucleotides, bicyclic nucleotides, mirror nucleotides, altritol nucleotides or nucleotides joined to an adjacent nucleotide by a 2′-5′ phosphodiester bond.

In one embodiment wherein each of 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (H), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In one preferred embodiment of Structure (H), x=y=19; three consecutive ribonucleotides at the 9-11 nucleotide positions of (N′)y comprise 2′-O-methyl modification and five consecutive ribonucleotides at the 3′ terminal position of (N′)x comprise 2′-O-methyl modification.

For all the above Structures (A)-(H), in various embodiments x=y and each of x and y is and integer selected from the group consisting of 19, 20, 21, 22 and 23. In certain embodiments, x=y=19. In other embodiments x=y=21. In additional embodiments the compounds of the invention comprise modified ribonucleotides in alternating positions wherein each N at the 5′ and 3′ termini of (N)x is modified in its sugar residue and the middle ribonucleotide is not modified, e.g. ribonucleotide in position 10 in a 19-mer strand, position 11 in a 21-mer and position 12 in a 23-mer strand.

In some embodiments where x=y=21 or x=y=23 the position of modifications in the 19-mer are adjusted for a 21- or 23-mer oligonucleotide with the proviso that the middle nucleotide of the antisense strand is preferably not modified.

In some embodiments, neither (N)x nor (N′)y are phosphorylated at the 3′ and 5′ termini. In other embodiments either or both (N)x and (N′)y are phosphorylated at the 3′ termini. In yet another embodiment, either or both (N)x and (N′)y are phosphorylated at the 3′ termini using non-cleavable phosphate groups. In yet another embodiment, either or both (N)x and (N′)y are phosphorylated at the terminal 5′ termini position using cleavable or non-cleavable phosphate groups. In some embodiments the siRNA compounds are blunt ended and are non-phosphorylated at the termini; however, comparative experiments have shown that siRNA compounds phosphorylated at one or both of the 3′-termini have similar activity in vivo compared to the non-phosphorylated compounds.

In certain embodiments for all the above-mentioned Structures, the siRNA compound is blunt ended, for example wherein both Z and Z′ are absent. In an alternative embodiment, the compound comprises at least one 3′ overhang, wherein at least one of Z or Z′ is present. Z and Z′ independently comprises one or more covalently linked modified or non-modified nucleotides, for example inverted dT or dA; dT, LNA, mirror nucleotide and the like. In some embodiments each of Z and Z′ are independently selected from dT and dTdT. siRNA in which Z and/or Z′ is present have similar activity and stability as siRNA in which Z and Z′ are absent.

In certain embodiments for all the above-mentioned Structures, the siRNA compound comprises one or more phosphonocarboxylate and/or phosphinocarboxylate nucleotides (PACE nucleotides). In some embodiments the PACE nucleotides are deoxyribonucleotides and the phosphinocarboxylate nucleotides are phosphinoacetate nucleotides.

In certain embodiments for all the above-mentioned Structures, the siRNA compound comprises one or more locked nucleic acids (LNA) also defined as bridged nucleic acids or bicyclic nucleotides. Preferred locked nucleic acids are 2′-O, 4′-C-ethylene nucleosides (ENA) or 2′-O, 4′-C-methylene nucleosides. Other examples of LNA and ENA nucleotides are disclosed in WO 98/39352, WO 00/47599 and WO 99/14226, all incorporated herein by reference.

In certain embodiments for all the above-mentioned Structures, the compound comprises one or more altritol monomers (nucleotides), also defined as 1,5 anhydro-2-deoxy-D-altrito-hexitol (see for example, Allart, et al., 1998. Nucleosides & Nucleotides 17:1523-1526; Herdewijn et al., 1999. Nucleosides & Nucleotides 18:1371-1376; Fisher et al., 2007, NAR 35(4):1064-1074; all incorporated herein by reference).

The present invention explicitly excludes compounds in which each of N and/or N′ is a deoxyribonucleotide (d-A, d-C, d-G, d-T). In certain embodiments (N)x and (N′)y may comprise independently 1, 2, 3, 4, 5, 6, 7, 8, 9 or more deoxyribonucleotides. In certain embodiments the present invention provides a compound wherein each of N is an unmodified ribonucleotide and the 3′ terminal nucleotide or 2, 3, 4, 5, 6, 7, 8, 9, 10, 11, 12, 13 or 14 consecutive nucleotides at the 3′ terminus of (N′)y are deoxyribonucleotides. In yet other embodiments each of N is an unmodified ribonucleotide and the 5′ terminal nucleotide or 2, 3, 4, 5, 6, 7, 8, 9, 10, 11, 12, 13 or 14 consecutive nucleotides at the 5′ terminus of (N′)y are deoxyribonucleotides. In further embodiments the 5′ terminal nucleotide or 2, 3, 4, 5, 6, 7, 8, or 9 consecutive nucleotides at the 5′ terminus and 1, 2, 3, 4, 5, or 6 consecutive nucleotides at the 3′ termini of (N)x are deoxyribonucleotides and each of N′ is an unmodified ribonucleotide. In yet further embodiments (N)x comprises unmodified ribonucleotides and 1 or 2, 3 or 4 consecutive deoxyribonucleotides independently at each of the 5′ and 3′ termini and 1 or 2, 3, 4, 5 or 6 consecutive deoxyribonucleotides in internal positions; and each of N′ is an unmodified ribonucleotide. In certain embodiments the 3′ terminal nucleotide or 2, 3, 4, 5, 6, 7, 8, 9, 10, 11, 12 13 or 14 consecutive nucleotides at the 3′ terminus of (N′)y and the terminal 5′ nucleotide or 2, 3, 4, 5, 6, 7, 8, 9, 10, 11, 12 13 or 14 consecutive nucleotides at the 5′ terminus of (N)x are deoxyribonucleotides. The present invention excludes compounds in which each of N and/or N′ is a deoxyribonucleotide. In some embodiments the 5′ terminal nucleotide of N or 2 or 3 consecutive of N and 1, 2, or 3 of N′ is a deoxyribonucleotide. Certain examples of active DNA/RNA siRNA chimeras are disclosed in US patent publication 2005/0004064, and Ui-Tei, 2008 (NAR 36(7):2136-2151) incorporated herein by reference in their entirety.

Unless otherwise indicated, in preferred embodiments of the structures discussed herein the covalent bond between each consecutive N and N′ is a phosphodiester bond.

A covalent bond refers to an internucleotide linkage linking one nucleotide monomer to an adjacent nucleotide monomer. A covalent bond includes for example, a phosphodiester bond, a phosphorothioate bond, a P-alkoxy bond, a P-carboxy bond and the like. The normal internucleoside linkage of RNA and DNA is a 3′ to 5′ phosphodiester linkage. In certain preferred embodiments a covalent bond is a phosphodiester bond. Covalent bond encompasses non-phosphorous-containing internucleoside linkages, such as those disclosed in WO 2004/041924 inter alia. Unless otherwise indicated, in preferred embodiments of the structures discussed herein the covalent bond between each consecutive N and N′ is a phosphodiester bond.

For all of the structures above, in some embodiments the oligonucleotide sequence of (N)x is fully complementary to the oligonucleotide sequence of (N′)y. In other embodiments (N)x and (N′)y are substantially complementary. In certain embodiments (N)x is fully complementary to a RTP801 mRNA. In other embodiments (N)x is substantially complementary to a RTP801 mRNA.

In some embodiments, neither (N)x nor (N′)y are phosphorylated at the 3′ and 5′ termini. In other embodiments either or both (N)x and (N′)y are phosphorylated at the 3′ termini (3′ Pi). In yet another embodiment, either or both (N)x and (N′)y are phosphorylated at the 3′ termini with non-cleavable phosphate groups. In yet another embodiment, either or both (N)x and (N′)y are phosphorylated at the terminal 2′ termini position using cleavable or non-cleavable phosphate groups. Further, the inhibitory nucleic acid molecules of the present invention may comprise one or more gaps and/or one or more nicks and/or one or more mismatches. Without wishing to be bound by theory, gaps, nicks and mismatches have the advantage of partially destabilizing the nucleic acid/siRNA, so that it may be more easily processed by endogenous cellular machinery such as DICER, DROSHA or RISC into its inhibitory components.

In the context of the present invention, a gap in a nucleic acid refers to the absence of one or more internal nucleotides in one strand, while a nick in a nucleic acid refers to the absence of an internucleotide linkage between two adjacent nucleotides in one strand. Any of the molecules of the present invention may contain one or more gaps and/or one or more nicks.

In one aspect the present invention provides a compound having Structure (I):

| | | |
|---|---|---|
| (I) | 5′ (N)x-Z 3′ | (antisense strand) |
| | 3′ Z′-(N′)y-z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
wherein x=18 to 27;
wherein y=18 to 27;
wherein (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide;
wherein in (N′)y at least one unconventional moiety is present, which unconventional moiety may be an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond; and
wherein the sequence of (N′)y is a sequence substantially complementary to (N)x; and wherein the sequence of (N)x comprises an antisense sequence substantially complementary to 18 to 27 consecutive ribonucleotides in a RTP801 mRNA. Preferably (N)x comprises an antisense sequence substantially identical to an antisense sequence set forth in any one of Tables A-I.

In some embodiments x=y=19. In other embodiments x=y=21. In some embodiments the at least one unconventional moiety is present at positions 15, 16, 17, or 18 in (N′)y. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18.

In other embodiments the unconventional moiety is an abasic moiety. In various embodiments (N′)y comprises at least five abasic ribose moieties or abasic deoxyribose moieties.

In yet other embodiments (N′)y comprises at least five abasic ribose moieties or abasic deoxyribose moieties and at least one of N′ is an LNA.

In some embodiments (N)x comprises nine alternating modified ribonucleotides. In other embodiments of Structure (I) (N)x comprises nine alternating modified ribonucleotides further comprising a 2′O modified nucleotide at position 2. In some embodiments (N)x comprises 2′O Me modified ribonucleotides at the odd numbered positions 1, 3, 5, 7, 9, 11, 13, 15, 17, 19. In other embodiments (N)x further comprises a 2′O Me modified ribonucleotide at one or both of positions 2 and 18. In yet other embodiments (N)x comprises 2′O Me modified ribonucleotides at positions 2, 4, 6, 8, 11, 13, 15, 17, 19.

In various embodiments z″ is present and is selected from an abasic ribose moiety, a deoxyribose moiety; an inverted abasic ribose moiety, a deoxyribose moiety; C6-amino-Pi; a mirror nucleotide.

In another aspect the present invention provides a compound having Structure (J) set forth below:

| | | |
|---|---|---|
| (J) | 5′ (N)x-Z 3′ | (antisense strand) |
| | 3′ Z′-(N′)y-z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent but if present is a capping moiety covalently attached at the 5′ terminus of (N′)y;
wherein x=18 to 27;
wherein y=18 to 27;
wherein (N)x comprises modified or unmodified ribonucleotides, and optionally at least one unconventional moiety;
wherein in (N′)y at least one unconventional moiety is present, which unconventional moiety may be an abasic ribose moiety, an abasic deoxyribose moiety, a modified or unmodified deoxyribonucleotide, a mirror nucleotide, a non-base pairing nucleotide analog or a nucleotide joined to an adjacent nucleotide by a 2′-5′ internucleotide phosphate bond; and
wherein the sequence of (N′)y is a sequence substantially complementary to (N)x; and wherein the sequence of (N)x comprises an antisense sequence substantially complementary to 18 to 27 consecutive ribonucleotides in an RTP801 mRNA. Preferably (N)x comprises an antisense sequence substantially identical to an antisense sequence set forth in any one of Tables A-I.

In some embodiments x=y=19. In other embodiments x=y=21. In some preferred embodiments (N)x comprises modified and unmodified ribonucleotides, and at least one unconventional moiety.

In some embodiments in (N)x the N at the 3′ terminus is a modified ribonucleotide and (N)x comprises at least 8 modified ribonucleotides. In other embodiments at least 5 of the at least 8 modified ribonucleotides are alternating beginning at the 3′ end. In some embodiments (N)x comprises an abasic moiety in one of positions 5, 6, 7, 8, 9, 10, 11, 12, 13, 14 or 15.

In some embodiments the at least one unconventional moiety in (N′)y is present at positions 15, 16, 17, or 18. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18. In other embodiments the at least one unconventional moiety in (N′)y is an abasic ribose moiety or an abasic deoxyribose moiety.

In yet another aspect the present invention provides a compound having Structure (K) set forth below:

| | | |
|---|---|---|
| (K) | 5′ (N)ₓ-Z 3′ | (antisense strand) |
| | 3′ Z′-(N′)_{y}-z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent but if present is a capping moiety covalently attached at the 5′ terminus of (N′)y;
wherein x=18 to 27;
wherein y=18 to 27;
wherein (N)x comprises a combination of modified or unmodified ribonucleotides and unconventional moieties, any modified ribonucleotide having a 2′-O-methyl on its sugar;
wherein (N′)y comprises modified or unmodified ribonucleotides and optionally an unconventional moiety, any modified ribonucleotide having a 2′OMe on its sugar;
wherein the sequence of (N′)y is a sequence substantially complementary to (N)x; and wherein the sequence of (N)x comprises an antisense sequence substantially complementary to 18 to 27 consecutive ribonucleotides in a RTP801 mRNA. Preferably (N)x comprises an antisense sequence substantially identical to an antisense sequence set forth in any one of Tables A-I.

In some embodiments x=y=19. In other embodiments x=y=21. In some preferred embodiments the at least one unconventional moiety is present in (N)x and is an abasic ribose moiety or an abasic deoxyribose moiety. In other embodiments the at least one unconventional moiety is present in (N)x and is a non-base pairing nucleotide analog. In various embodiments (N′)y comprises unmodified ribonucleotides. In some embodiments (N)x comprises at least five abasic ribose moieties or abasic deoxyribose moieties or a combination thereof. In certain embodiments (N)x and/or (N′)y comprise modified ribonucleotides which do not base pair with corresponding modified or unmodified ribonucleotides in (N′)y and/or (N)x.

In various embodiments the present invention provides an siRNA set forth in Structure (L):

| | | |
|---|---|---|
| (L) | 5′ (N)ₓ-Z 3′ | (antisense strand) |
| | 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein x=y=19;
wherein in (N′)y the nucleotide in at least one of positions 15, 16, 17, 18 and 19 comprises a nucleotide selected from an abasic unconventional moiety, a mirror nucleotide, a deoxyribonucleotide and a nucleotide joined to an adjacent nucleotide by a 2′-5′ internucleotide bond;
wherein (N)x comprises alternating modified ribonucleotides and unmodified ribonucleotides each modified ribonucleotide being modified so as to have a 2′-O-methyl on its sugar and the ribonucleotide located at the middle position of (N)x being modified or unmodified, preferably unmodified; and
wherein the sequence of (N′)y is a sequence substantially complementary to (N)x; and wherein the sequence of (N)x comprises an antisense sequence substantially complementary to 18 to 27 consecutive ribonucleotides in a RTP801 mRNA. Preferably (N)x comprises an antisense sequence substantially identical to an antisense sequence set forth in any one of Tables A-I.

In some embodiments of Structure (L), in (N′)y the nucleotide in one or both of positions 17 and 18 comprises a modified nucleotide selected from an abasic unconventional moiety, a mirror nucleotide and a nucleotide joined to an adjacent nucleotide by a 2′-5′ internucleotide bond. In some embodiments the mirror nucleotide is selected from L-DNA and L-RNA. In various embodiments the mirror nucleotide is L-DNA.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide or pseudo nucleotide at position 2 wherein the pseudo nucleotide may be an abasic unconventional moiety and the modified nucleotide is optionally a mirror nucleotide.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)x further comprises 2′O-Me modified ribonucleotides at one or both positions 2 and 18. In other embodiments (N)x comprises 2′O Me modified ribonucleotides at positions 2, 4, 6, 8, 11, 13, 15, 17, 19.

Other embodiments of Structures (L) are envisaged wherein x=y=21; in these embodiments the modifications for (N′)y discussed above instead of being in positions 17 and 18 are in positions 19 and 20 for 21-mer oligonucleotide; similarly the modifications in positions 15, 16, 17, 18 or 19 are in positions 17, 18, 19, 20 or 21 for the 21-mer oligonucleotide. The 2′O Me modifications on the antisense strand are similarly adjusted. In some embodiments (N)x comprises 2′O Me modified ribonucleotides at the odd numbered positions (5′ to 3′; positions 1, 3, 5, 7, 9, 12, 14, 16, 18, 20 for the 21 mer oligonucleotide [nucleotide at position 11 unmodified]). In other embodiments (N)x comprises 2′OMe modified ribonucleotides at positions 2, 4, 6, 8, 10, 12, 14, 16, 18, 20 [nucleotide at position 11 unmodified] for the 21 mer oligonucleotide.

In some embodiments (N′)y further comprises a 5′ terminal cap nucleotide. In various embodiments the terminal cap moiety is selected from an abasic unconventional moiety, an inverted abasic unconventional moiety, an L-DNA nucleotide, and a C6-imine phosphate (C6 amino linker with phosphate at terminus).

In other embodiments the present invention provides a compound having Structure (M) set forth below:

| | | |
|---|---|---|
| (M) | 5′ (N)ₓ-Z 3′ | (antisense strand) |
| | 3′ Z′-(N′)_{y} 5′ | (sense strand) |

wherein each of N and N′ is selected from a pseudo-nucleotide and a nucleotide;
wherein each nucleotide is selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent; wherein x=18 to 27;
wherein y=18 to 27;
wherein the sequence of (N′)y is a sequence substantially complementary to (N)x; and wherein the sequence of (N)x comprises an antisense sequence substantially complementary to 18 to 27 consecutive ribonucleotides in a RTP801 mRNA. Preferably (N)x comprises an antisense sequence substantially identical to an antisense sequence set forth in any one of Tables A-I.

In other embodiments the present invention provides a double stranded compound having Structure (N) set forth below:

| | | |
|---|---|---|
| (N) | 5′ (N)ₓ-Z 3′ | (antisense strand) |
| | 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein each of x and y is independently an integer between 18 and 40;
wherein (N)x, (N′)y or (N)x and (N′)y comprise non base-pairing modified nucleotides such that (N)x and (N′)y form less than 15 base pairs in the double stranded compound; and wherein the sequence of (N′)y is a sequence substantially complementary to (N)x; and wherein the sequence of (N)x comprises an antisense sequence substantially complementary to 18 to 40 consecutive ribonucleotides in a RTP801 mRNA. Preferably (N)x comprises an antisense sequence substantially identical to an antisense sequence set forth in any one of Tables A-I.

In other embodiments the present invention provides a compound having Structure (O) set forth below:

| | | |
|---|---|---|
| (O) | 5′ (N)ₓ-Z 3′ | (antisense strand) |
| | 3′ Z′-(N′)_{y} 5′ | (sense strand) |

wherein each of N is a nucleotide selected from an unmodified ribonucleotide, a modified ribonucleotide, an unmodified deoxyribonucleotide and a modified deoxyribonucleotide;
wherein each of N′ is a nucleotide analog selected from a six membered sugar nucleotide, seven membered sugar nucleotide, morpholino moiety, peptide nucleic acid and combinations thereof;
wherein each of (N)ₓ and (N′)_{y} is an oligonucleotide in which each consecutive N or N′ is joined to the next N or N′ by a covalent bond;
wherein Z and Z′ are absent;
wherein each of x and y is independently an integer between 18 and 40;
wherein the sequence of (N′)y is a sequence substantially complementary to (N)x; and wherein the sequence of (N)x comprises an antisense sequence substantially complementary to 18 to 27 consecutive ribonucleotides in a RTP801 mRNA. Preferably (N)x comprises an antisense sequence substantially identical to an antisense sequence set forth in any one of Tables A-I.

In other embodiments the present invention provides a compound having Structure (P) set forth below:

| | | |
|---|---|---|
| (P) | 5′ (N)ₓ-Z 3′ | (antisense strand) |
| | 3′ Z′-(N′)_{y} 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein each of x and y is independently an integer between 18 and 40;
wherein one of N or N′ in an internal position of (N)x or (N′)y or one or more of N or N′ at a terminal position of (N)x or (N′)y comprises an abasic moiety or a 2′ modified nucleotide;
wherein the sequence of (N′)y is a sequence substantially complementary to (N)x; and wherein the sequence of (N)x comprises an antisense sequence substantially complementary to 18 to 27 consecutive ribonucleotides in a RTP801 mRNA. Preferably (N)x comprises an antisense sequence substantially identical to an antisense sequence set forth in any one of Tables A-I.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide at position 2 wherein the modified nucleotide is selected from a mirror nucleotide and an abasic unconventional moiety.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)x further comprises 2′O-Me modified ribonucleotides at one or both positions 2 and 18. In other embodiments (N)x comprises 2′OMe modified ribonucleotides at positions 2, 4, 6, 8, 11, 13, 15, 17, 19.

An additional novel molecule provided by the present invention is an oligonucleotide comprising consecutive nucleotides wherein a first segment of such nucleotides encode a first inhibitory RNA molecule, a second segment of such nucleotides encode a second inhibitory RNA molecule, and a third segment of such nucleotides encode a third inhibitory RNA molecule. Each of the first, the second and the third segment may comprise one strand of a double stranded RNA and the first, second and third segments may be joined together by a linker. Further, the oligonucleotide may comprise three double stranded segments joined together by one or more linker.

Thus, one molecule provided by the present invention is an oligonucleotide comprising consecutive nucleotides which encode three inhibitory RNA molecules; said oligonucleotide may possess a triple stranded structure, such that three double stranded arms are linked together by one or more linker, such as any of the linkers presented hereinabove. This molecule forms a “star”-like structure, and may also be referred to herein as RNAstar. Such structures are disclosed in PCT patent publication WO 2007/091269, assigned to the assignee of the present invention and incorporated herein in its entirety by reference.

Said triple-stranded oligonucleotide may be an oligoribonucleotide having the general structure:

| | | |
|---|---|---|
| 5′ Oligo1 (sense) | LINKER A | Oligo2 (sense) 3′ |
| 3′ Oligo1 (antisense) | LINKER B | Oligo3 (sense) 5′ |
| 3′ Oligo3 (antisense) | LINKER C | Oligo2 (antisense) 5′ |
| Or | | |
| 5′ Oligo1 (sense) | LINKER A | Oligo2 (antisense) 3′ |
| 3′ Oligo1 (antisense) | LINKER B | Oligo3 (sense) 5′ |
| 3′ Oligo3 (antisense) | LINKER C | Oligo2 (sense) 5′ |
| or | | |
| 5′ Oligo1 (sense) | LINKER A | Oligo3 (antisense) 3′ |
| 3′ Oligo1 (antisense) | LINKER B | Oligo2 (sense) 5′ |
| 5′ Oligo3 (sense) | LINKER C | Oligo2 (antisense) 3′ |

wherein one or more of linker A, linker B or linker C is present; any combination of two or more oligonucleotides and one or more of linkers A-C is possible, so long as the polarity of the strands and the general structure of the molecule remains. Further, if two or more of linkers A-C are present, they may be identical or different.

Thus, a triple-armed structure is formed, wherein each arm comprises a sense strand and complementary antisense strand (i.e. Oligo1 antisense base pairs to Oligo1 sense etc.). The triple armed structure may be triple stranded, whereby each arm possesses base pairing.

Further, the above triple stranded structure may have a gap instead of a linker in one or more of the strands. Such a molecule with one gap is technically quadruple stranded and not triple stranded; inserting additional gaps or nicks will lead to the molecule having additional strands. Preliminary results obtained by the inventors of the present invention indicate that said gapped molecules are more active in inhibiting the RTP801 target gene than the similar but non-gapped molecules.

In some embodiments, neither antisense nor sense strands of the novel siRNA compounds of the invention are phosphorylated at the 3′ and 5′ termini. In other embodiments either or both antisense and sense strands are phosphorylated at the 3′ termini. In yet another embodiment, either or both antisense and sense strands are phosphorylated at the 3′ termini using non-cleavable phosphate groups. In yet another embodiment, either or both antisense and sense strands are phosphorylated at the terminal 5′ termini position using cleavable or non-cleavable phosphate groups. In yet another embodiment, either or both antisense and sense strands are phosphorylated at the terminal 2′ termini position using cleavable or non-cleavable phosphate groups. In some embodiments the siRNA compounds are blunt ended and are non-phosphorylated at the termini; however, comparative experiments have shown that siRNA compounds phosphorylated at one or both of the 3′-termini have similar activity in vivo compared to the non-phosphorylated compounds.

Any siRNA sequence disclosed herein can be prepared having any of the modifications/Structures disclosed herein. The combination of sequence plus structure is novel and can be used in the treatment of the conditions disclosed herein.

Unless otherwise indicated, in preferred embodiments of the structures discussed herein the covalent bond between each consecutive N and N′ is a phosphodiester bond.

For all of the structures above, in some embodiments the oligonucleotide sequence of antisense strand is fully complementary to the oligonucleotide sequence of sense. In other embodiments the antisense and sense strands are substantially complementary. In certain embodiments the antisense strand is fully complementary to a RTP801 mRNA. In other embodiments the antisense strand is substantially complementary to a RTP801 mRNA. Preferably the sequence of the antisense strand is substantially identical to an antisense sequence set forth in any one of Tables A-I.

In some embodiments the present invention provides an expression vector comprising an antisense oligonucleotide disclosed in any one of Tables E-I. In some embodiments the expression vector further comprises a sense oligonucleotide having complementarity to the antisense oligonucleotide. In various embodiments the present invention further provides a cell comprising an expression vector comprising an antisense oligonucleotide disclosed in any one of Tables E-I. The present invention further provides a siRNA expressed in a cell comprising an expression vector comprising an antisense oligonucleotide disclosed in any one of Tables E-I, a pharmaceutical composition comprising same and use thereof for treatment of any one of the diseases and disorders disclosed herein.

In other embodiments the present invention provides a first expression vector comprising an antisense oligonucleotide disclosed in any one of Tables E-I and a second expression vector comprising a sense oligonucleotide having complementarity to the antisense oligonucleotide comprised in the first expression vector. In various embodiments the present invention further provides a cell comprising a first expression vector comprising an antisense oligonucleotide disclosed in any one of Tables E-I and a second expression vector comprising a sense oligonucleotide having complementarity to the antisense oligonucleotide comprised in the first expression vector. The present invention further provides a siRNA expressed in a cell comprising such first and second expression vector, a pharmaceutical composition comprising same and use thereof for treatment of any one of the diseases and disorders disclosed herein.

siRNA Synthesis

Using proprietary algorithms and the known sequence of RTP801 gene disclosed herein, the sequences of many potential siRNAs are generated. siRNA molecules according to the above specifications are prepared essentially as described herein.

The siRNA compounds of the present invention are synthesized by any of the methods that are well known in the art for synthesis of ribonucleic (or deoxyribonucleic) oligonucleotides. Such synthesis is, among others, described in Beaucage and Iyer, Tetrahedron 1992; 48:2223-2311; Beaucage and Iyer, Tetrahedron 1993; 49: 6123-6194 and Caruthers, et. al., Methods Enzymol. 1987; 154: 287-313; the synthesis of thioates is, among others, described in Eckstein, Ann. Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et al., in IRL Press 1989 edited by Oliver R. W. A.; Kap. 7: 183-208.

Other synthetic procedures are known in the art, e.g. the procedures described in Usman et al., 1987, J. Am. Chem. Soc., 109, 7845; Scaringe et al., 1990, NAR., 18, 5433; Wincott et al., 1995, NAR. 23, 2677-2684; and Wincott et al., 1997, Methods Mol. Bio., 74, 59, may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., 1992, Science 256, 9923; Draper et al., International Patent Publication No. WO 93/23569; Shabarova et al., 1991, NAR 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via tandem synthesis methodology, as described for example in US Patent Publication No. US 2004/0019001, wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker is selected from a polynucleotide linker or a non-nucleotide linker.

### Pharmaceutical Compositions

While it is possible for the compounds of the present invention to be administered as the raw chemical, it is preferable to present them as a pharmaceutical composition. Accordingly the present invention provides a pharmaceutical composition comprising one or more of the chemically modified siRNA compounds of the invention; and a pharmaceutically acceptable carrier. In some embodiments the pharmaceutical composition comprises two or more novel siRNA compounds of the invention.

The invention further provides a pharmaceutical composition comprising at least one compound of the invention covalently or non-covalently bound to one or more compounds of the invention in an amount effective to inhibit the RTP801 gene; and a pharmaceutically acceptable carrier. In some embodiments the siRNA compounds are processed intracellularly by endogenous cellular complexes to produce one or more oligoribonucleotides of the invention.

The invention further provides a pharmaceutical composition comprising a pharmaceutically acceptable carrier and one or more of the chemically modified siRNA compounds of the invention in an amount effective to inhibit expression in a cell of a RTP801 gene, the compound comprising a sequence which is substantially complementary to the sequence of RTP801 mRNA.

In some embodiments, the siRNA compounds according to the present invention are the main active component in a pharmaceutical composition. In other embodiments the siRNA compounds according to the present invention are one of the active components of a pharmaceutical composition containing two or more siRNAs, said pharmaceutical composition further being comprised of one or more additional siRNA molecule which targets the RTP801 gene. In other embodiments the siRNA compounds according to the present invention are one of the active components of a pharmaceutical composition containing two or more siRNAs, said pharmaceutical composition further being comprised of one or more additional siRNA molecule which targets one or more additional gene. In some embodiments, simultaneous inhibition of RTP801 gene by two or more siRNA compounds of the invention provides additive or synergistic effect for treatment of the diseases disclosed herein. In some embodiments, simultaneous inhibition of RTP801 gene and said additional gene(s) provides additive or synergistic effect for treatment of the diseases disclosed herein.

In some embodiments, the siRNA compounds disclosed herein are linked or bound (covalently or non-covalently) to an antibody or aptamer against cell surface internalizable molecules expressed on the target cells, in order to achieve enhanced targeting for treatment of the diseases disclosed herein. In one specific embodiment, anti-Fas antibody (preferably a neutralizing antibody) is combined (covalently or non-covalently) with a siRNA compound according to the present invention. In various embodiments, an aptamer which acts like a ligand/antibody is combined (covalently or non-covalently) with a siRNA compound according to the present invention.

### RNA Interference

A number of PCT applications have recently been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

RNA interference (RNAi) is based on the ability of dsRNA species to enter a cytoplasmic protein complex, where it is then targeted to the complementary cellular RNA and specifically degrade it. The RNA interference response features an endonuclease complex containing a siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir et al., Genes Dev., 2001, 15(2):188-200). In more detail, longer dsRNAs are digested into short (17-29 bp) dsRNA fragments (also referred to as short inhibitory RNAs, “siRNAs”) by type III RNAses (DICER, DROSHA, etc.; Bernstein et al., Nature, 2001, 409(6818):363-6; Lee et al., Nature, 2003, 425(6956):415-9). The RISC protein complex recognizes these fragments and complementary mRNA. The whole process is culminated by endonuclease cleavage of target mRNA (McManus & Sharp, Nature Rev Genet, 2002, 3(10):737-47; Paddison & Hannon, Curr Opin Mol. Ther. 2003, 5(3):217-24). (For additional information on these terms and proposed mechanisms, see for example Bernstein et al., RNA 2001, 7(11):1509-21; Nishikura, Cell 2001, 107(4):415-8 and PCT publication WO 01/36646).

The selection and synthesis of siRNA corresponding to known genes has been widely reported; see for example Ui-Tei et al., J Biomed Biotechnol. 2006; 2006: 65052; Chalk et al., BBRC. 2004, 319(1): 264-74; Sioud & Leirdal, Met. Mol. Biol.; 2004, 252:457-69; Levenkova et al., Bioinform. 2004, 20(3):430-2; Ui-Tei et al., Nuc. Acid Res. 2004, 32(3):936-48. For examples of the use of, and production of, modified siRNA see Braasch et al., Biochem., 2003, 42(26):7967-75; Chiu et al., RNA, 2003, 9(9):1034-48; PCT publications WO 2004/015107 (Atugen); WO 02/44321 (Tuschl et al), and U.S. Pat. Nos. 5,898,031 and 6,107,094.

### Delivery

The chemically modified siRNA compound of the invention can is administered as the compound per se (i.e. as naked siRNA) or as pharmaceutically acceptable salt and is administered alone or as an active ingredient in combination with one or more pharmaceutically acceptable carrier, solvent, diluent, excipient, adjuvant and vehicle. In some embodiments, the siRNA molecules of the present invention are delivered to the target tissue by direct application of the naked molecules prepared with a carrier or a diluent.

The term “naked siRNA” refers to siRNA molecules that are free from any delivery vehicle that acts to assist, promote or facilitate entry into the cell, including viral sequences, viral particles, liposome formulations, lipofectin or precipitating agents and the like. For example, siRNA in PBS is “naked siRNA”.

Pharmaceutically acceptable carriers, solvents, diluents, excipients, adjuvants and vehicles as well as implant carriers generally refer to inert, non-toxic solid or liquid fillers, diluents or encapsulating material not reacting with the active siRNA compounds of the invention and they include liposomes and microspheres. Formulating the compositions in liposomes may benefit absorption. Additionally, the compositions may include a PFC liquid such as perflubron, and the compositions may be formulated as a complex of the compounds of the invention with polyethylemeimine (PEI). Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many such implants, delivery systems, and modules are well known to those skilled in the art. In one specific embodiment of this invention topical and transdermal formulations are selected.

Accordingly, in some embodiments the siRNA molecules of the invention are delivered in liposome formulations and lipofectin formulations and the like and can be prepared by methods well known to those skilled in the art. Such methods are described, for example, in U.S. Pat. Nos. 5,593,972, 5,589,466, and 5,580,859, which are herein incorporated by reference.

Delivery systems aimed specifically at the enhanced and improved delivery of siRNA into mammalian cells have been developed (see, for example, Shen et al FEBS Let. 539: 111-114 (2003), Xia et al., Nat. Biotech. 20: 1006-1010 (2002), Reich et al., Mol. Vision. 9: 210-216 (2003), Sorensen et al., J. Mol. Biol. 327: 761-766 (2003), Lewis et al., Nat. Gen. 32: 107-108 (2002) and Simeoni et al., NAR 31, 11: 2717-2724 (2003)). siRNA has recently been successfully used for inhibition of gene expression in primates; (for details see for example, Tolentino et al., Retina 2004.24(1):132-138).

Additional formulations for improved delivery of the compounds of the present invention can include non-formulated compounds, compounds covalently bound to cholesterol, and compounds bound to targeting antibodies (Song et al., Antibody mediated in vivo delivery of small interfering RNAs via cell-surface receptors, Nat. Biotechnol. 2005. 23(6):709-17). Cholesterol-conjugated siRNAs (and other steroid and lipid conjugated siRNAs) can been used for delivery (see for example Soutschek et al Nature. 2004. 432:173-177; and Lorenz et al. Bioorg. Med. Chem. Lett. 2004. 14:4975-4977).

The naked siRNA or the pharmaceutical compositions comprising the chemically modified siRNA of the present invention are administered and dosed in accordance with good medical practice, taking into account the clinical condition of the individual patient, the disease to be treated, the site and method of administration, scheduling of administration, patient age, sex, body weight and other factors known to medical practitioners.

A “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art. The siRNA of the invention can be administered in a single dose or in multiple doses.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-4 weeks or longer.

The chemically modified siRNA compounds of the present invention can be administered by any of the conventional routes of administration. The chemically modified siRNA compounds are administered orally, subcutaneously or parenterally including intravenous, intraarterial, intramuscular, intraperitoneally, intraocular, transtympanic and intranasal administration as well as intrathecal and infusion techniques. Implants of the compounds are also useful.

Liquid forms are prepared for invasive administration, e.g. injection or for topical or local administration. The term injection includes subcutaneous, transdermal, intravenous, intramuscular, intrathecal, intraocular, transtympanic and other parental routes of administration. The liquid compositions include aqueous solutions, with and without organic co-solvents, aqueous or oil suspensions, emulsions with edible oils, as well as similar pharmaceutical vehicles. In a particular embodiment, the administration comprises intravenous administration. In some embodiments the compounds of the present invention are formulated as eardrops for topical administration to the ear. In some embodiments the compounds of the present invention are formulated as eye drops for topical administration to the surface of the eye. Further information on administration of the compounds of the present invention can be found in Tolentino et al., Retina 2004. 24:132-138; and Reich et al., Molecular Vision, 2003. 9:210-216.

In addition, in certain embodiments the compositions for use in the novel treatments of the present invention are formed as aerosols, for example for intranasal administration. In certain embodiments the compositions for use in the novel treatments of the present invention are formed as nasal drops, for example for intranasal instillation.

The therapeutic compositions of the present invention are preferably administered into the lung by inhalation of an aerosol containing these compositions/compounds, or by intranasal or intratracheal instillation of said compositions. For further information on pulmonary delivery of pharmaceutical compositions see Weiss et al., Human Gene Therapy 1999. 10:2287-2293; Densmore et al., Molecular therapy 1999. 1:180-188; Gautam et al., Molecular Therapy 2001. 3:551-556; and Shahiwala & Misra, AAPS PharmSciTech 2004. 24; 6(3):E482-6. Additionally, respiratory formulations for siRNA are described in U.S. Patent Application Publication No. 2004/0063654. Respiratory formulations for siRNA are described in US Patent Application Publication No. 2004/0063654.

In certain embodiments, oral compositions (such as tablets, suspensions, solutions) may be effective for local delivery to the oral cavity such as oral composition suitable for mouthwash for the treatment of oral mucositis.

In a particular embodiment, the chemically modified siRNA compounds of the invention are formulated for intravenous administration for delivery to the kidney for the treatment of kidney disorders, e.g. acute renal failure (ARF), delayed graft function (DGF). It is noted that the delivery of the siRNA compounds according to the present invention to the target cells in the kidney proximal tubules is particularly effective in the treatment of ARF and DGF. Without being bound by theory, this may be due to the fact that normally siRNA molecules are excreted from the body via the cells of the kidney proximal tubules. Thus, naked siRNA molecules concentrate in the cells that are targeted for the therapy in ARF and DGF.

Delivery of compounds into the brain is accomplished by several methods such as, inter alia, neurosurgical implants, blood-brain barrier disruption, lipid mediated transport, carrier mediated influx or efflux, plasma protein-mediated transport, receptor-mediated transcytosis, absorptive-mediated transcytosis, neuropeptide transport at the blood-brain barrier, and genetically engineering “Trojan horses” for drug targeting. The above methods are performed, for example, as described in “Brain Drug Targeting: the future of brain drug development”, W. M. Pardridge, Cambridge University Press, Cambridge, UK (2001).

In addition, in certain embodiments the compositions for use in the novel treatments of the present invention are formed as aerosols, for example for intranasal administration.

Intranasal delivery for the treatment of CNS diseases has been attained with acetylcholinesterase inhibitors such as galantamine and various salts and derivatives of galantamine, for example as described in US Patent Application Publication No. 2006003989 and PCT Applications Publication Nos. WO 2004/002402 and WO 2005/102275. Intranasal delivery of nucleic acids for the treatment of CNS diseases, for example by intranasal instillation of nasal drops, has been described, for example, in PCT Application Publication No. WO 2007/107789.

### Methods of Treatment

In one aspect the present invention relates to a method of treating a subject suffering from a disorder associated with RTP801 comprising administering to the subject a therapeutically effective amount of an siRNA compound of the present invention. In preferred embodiments the subject being treated is a warm-blooded animal and, in particular, mammal including human.

“Treating a subject” refers to administering to the subject a therapeutic substance effective to ameliorate symptoms associated with a disease, to lessen the severity or cure the disease, or to prevent the disease from occurring. “Treatment” refers to both therapeutic treatment and prophylactic or preventative measures, wherein the object is to prevent a disorder or reduce the symptoms of a disorder. Those in need of treatment include those already experiencing the disease or condition, those prone to having the disease or condition, and those in which the disease or condition is to be prevented. The compounds of the invention are administered before, during or subsequent to the onset of the disease or condition.

A “therapeutically effective dose” refers to an amount of a pharmaceutical compound or composition which is effective to achieve an improvement in a subject or his physiological systems including, but not limited to, improved survival rate, more rapid recovery, or improvement or elimination of symptoms, and other indicators as are selected as appropriate determining measures by those skilled in the art.

The methods of treating the diseases disclosed herein and included in the present invention may include administering a RTP801 siRNA inhibitor in conjunction or in combination with an additional RTP801 inhibitor, a substance which improves the pharmacological properties of the active ingredient (e.g. siRNA) as detailed below, or an additional compound known to be effective in the treatment of a subject suffering from or susceptible to any of the hereinabove mentioned diseases and disorders, such as macular degeneration, COPD, ARF, DR, inter alia. The present invention thus provides in another aspect, a combination of a therapeutic siRNA compound of the invention together with at least one additional therapeutically active agent. By “in conjunction with” or “in combination with” is meant prior to, simultaneously or subsequent to. Accordingly, the individual components of such a combination can be administered either sequentially or simultaneously from the same or separate pharmaceutical formulations. Further detail on exemplary combination therapies is given below.

“Respiratory disorder” refers to conditions, diseases or syndromes of the respiratory system including but not limited to pulmonary disorders of all types including chronic obstructive pulmonary disease (COPD), emphysema, chronic bronchitis, asthma and lung cancer, inter alia. Emphysema and chronic bronchitis may occur as part of COPD or independently.

“Microvascular disorder” refers to any condition that affects microscopic capillaries and lymphatics, in particular vasospastic diseases, vasculitic diseases and lymphatic occlusive diseases. Examples of microvascular disorders include, inter alia: eye disorders such as Amaurosis Fugax (embolic or secondary to SLE), apla syndrome, Prot CS and ATIII deficiency, microvascular pathologies caused by IV drug use, dysproteinemia, temporal arteritis, anterior ischemic optic neuropathy, optic neuritis (primary or secondary to autoimmune diseases), glaucoma, von Hippel Lindau syndrome, corneal disease, corneal transplant rejection cataracts, Eales' disease, frosted branch angiitis, encircling buckling operation, uveitis including pars planitis, choroidal melanoma, choroidal hemangioma, optic nerve aplasia; retinal conditions such as retinal artery occlusion, retinal vein occlusion, retinopathy of prematurity, HIV retinopathy, Purtscher retinopathy, retinopathy of systemic vasculitis and autoimmune diseases, diabetic retinopathy, hypertensive retinopathy, radiation retinopathy, branch retinal artery or vein occlusion, idiopathic retinal vasculitis, aneurysms, neuroretinitis, retinal embolization, acute retinal necrosis, Birdshot retinochoroidopathy, long-standing retinal detachment; systemic conditions such as Diabetes mellitus, diabetic retinopathy (DR), diabetes-related microvascular pathologies (as detailed herein), hyperviscosity syndromes, aortic arch syndromes and ocular ischemic syndromes, carotid-cavernous fistula, multiple sclerosis, systemic lupus erythematosus, arteriolitis with SS-A autoantibody, acute multifocal hemorrhagic vasculitis, vasculitis resulting from infection, vasculitis resulting from Behçet's disease, sarcoidosis, coagulopathies, neuropathies, nephropathies, microvascular diseases of the kidney, and ischemic microvascular conditions, inter alia.

Microvascular disorders may comprise a neovascular element. The term “neovascular disorder” refers to those conditions where the formation of blood vessels (neovascularization) is harmful to the patient. Examples of ocular neovascularization include: retinal diseases (diabetic retinopathy, diabetic Macular Edema, chronic glaucoma, retinal detachment, and sickle cell retinopathy); rubeosis iritis; proliferative vitreo-retinopathy; inflammatory diseases; chronic uveitis; neoplasms (retinoblastoma, pseudoglioma and melanoma); Fuchs' heterochromic iridocyclitis; neovascular glaucoma; corneal neovascularization (inflammatory, transplantation and developmental hypoplasia of the iris); neovascularization following a combined vitrectomy and lensectomy; vascular diseases (retinal ischemia, choroidal vascular insufficiency, choroidal thrombosis and carotid artery ischemia); neovascularization of the optic nerve; and neovascularization due to penetration of the eye or contusive ocular injury. All these neovascular conditions may be treated using the compounds and pharmaceutical compositions of the present invention.

“Eye disease” refers to conditions, diseases or syndromes of the eye including but not limited to any conditions involving choroidal neovascularization (CNV), wet and dry AMD, ocular histoplasmosis syndrome, angiod streaks, ruptures in Bruch's membrane, myopic degeneration, ocular tumors, retinal degenerative diseases and retinal vein occlusion (RVO). Some conditions disclosed herein, such as DR, which may be treated according to the methods of the present invention have been regarded as either a microvascular disorder and an eye disease, or both, under the definitions presented herein.

More specifically, the present invention provides methods and compositions useful in treating a subject suffering from or susceptible to adult respiratory distress syndrome (ARDS); Chronic obstructive pulmonary disease (COPD); acute lung injury (ALI); Emphysema; Diabetic Neuropathy, nephropathy and retinopathy; diabetic macular edema (DME) and other diabetic conditions; Glaucoma; age related macular degeneration (AMD); bone marrow transplantation (BMT) retinopathy; ischemic conditions; ocular ischemic syndrome (OIS); kidney disorders: acute renal failure (ARF), delayed graft function (DGF), transplant rejection; hearing disorders (including hearing loss); spinal cord injuries; oral mucositis; dry eye syndrome and pressure sores; neurological disorders arising from ischemic or hypoxic conditions, such as hypertension, hypertensive cerebral vascular disease, a constriction or obstruction of a blood vessel—as occurs in the case of a thrombus or embolus, angioma, blood dyscrasias, any form of compromised cardiac function including cardiac arrest or failure, systemic hypotension; stroke, epilepsy, neurodegenerative disorders, including, without being limited to Parkinson's disease, Amyotrophic Lateral Sclerosis (ALS, Lou Gehrig's Disease), Alzheimer's disease, Huntington's disease and any other disease-induced dementia (such as HIV-associated dementia for example).

Additionally, the invention provides a method of down-regulating the expression of a RTP801 gene by at least 50% as compared to a control comprising contacting RTP801 mRNA with one or more of the chemically modified siRNA compounds of the present invention.

In one embodiment the chemically modified siRNA compound of the present invention down-regulates the mammalian RTP801 gene whereby the down-regulation is selected from the group comprising down-regulation of gene function, down-regulation of polypeptide and down-regulation of mRNA expression.

The invention provides a method of inhibiting the expression of the RTP801 gene by at least 40%, preferably by 50%, 60% or 70%, more preferably by 75%, 80% or 90% as compared to a control comprising contacting an mRNA transcript of the RTP801 gene with one or more of the siRNA compounds of the invention.

In one embodiment the chemically modified siRNA compound of the invention inhibits the RTP801 polypeptide, whereby the inhibition is selected from the group comprising inhibition of function (which is examined by, for example, an enzymatic assay or a binding assay with a known interactor of the native gene/polypeptide, inter alia), inhibition of RTP801 protein (which is examined by, for example, Western blotting, ELISA or immuno-precipitation, inter alia) and inhibition of RTP801 mRNA expression (which is examined by, for example, Northern blotting, quantitative RT-PCR, in-situ hybridization or microarray hybridization, inter alia).

In one embodiment the chemically modified siRNA compound of the invention is down-regulating RTP801 gene or polypeptide, whereby the down-regulation is selected from the group comprising down-regulation of function (which is examined by, for example, an enzymatic assay or a binding assay with a known interactor of the native gene/polypeptide, inter alia), down-regulation of protein (which is examined by, for example, Western blotting, ELISA or immuno-precipitation, inter alia) and down-regulation of RTP801 mRNA expression (which is examined by, for example, Northern blotting, quantitative RT-PCR, in-situ hybridization or microarray hybridization, inter alia).

In additional embodiments the invention provides a method of treating a subject suffering from or susceptible to any disease or disorder accompanied by an elevated level of a mammalian RTP801 gene, the method comprising administering to the subject a chemically modified siRNA compound or composition of the invention in a therapeutically effective dose thereby treating the subject.

The present invention relates to the use of compounds which down-regulate the expression of a mammalian RTP801 gene particularly to novel small interfering RNAs (siRNAs), in the treatment of the following diseases or conditions in which inhibition of the expression of the mammalian RTP801 gene is beneficial: ARDS; COPD; ALI; Emphysema; Diabetic Neuropathy, nephropathy and retinopathy; DME and other diabetic conditions; Glaucoma; AMD; BMT retinopathy; ischemic conditions including stroke; OIS; Neurodegenerative disorders such as Parkinson's, Alzheimer's, ALS; kidney disorders: ARF, DGF, transplant rejection; hearing disorders; spinal cord injuries; oral mucositis; dry eye syndrome and pressure sores.

Methods, novel chemically modified siRNA molecules and pharmaceutical compositions comprising said siRNA compounds which inhibit a mammalian RTP801 gene or polypeptide are discussed herein at length, and any of said siRNA molecules and/or pharmaceutical compositions are beneficially employed in the treatment of a subject suffering from or susceptible to any of said conditions. It is to be explicitly understood that known compounds are excluded from the present invention. Novel methods of treatment using known compounds and compositions fall within the scope of the present invention.

The method of the invention includes administering a therapeutically effective amount of one or more of the chemically modified siRNA compounds of the invention which down-regulate expression of a RTP801 gene.

By “exposure to a toxic agent” is meant that the toxic agent is made available to, or comes into contact with, a mammal. A toxic agent can be toxic to the nervous system. Exposure to a toxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

In other embodiments the chemically modified siRNA compounds and methods of the invention are useful for treating or preventing the incidence or severity of other diseases and conditions in a subject. These diseases and conditions include, but are not limited to stroke and stroke-like situations (e.g. cerebral, renal, cardiac failure), neuronal cell death, brain injuries with or without reperfusion, chronic degenerative diseases e.g. neurodegenerative disease including, Huntington's disease, multiple sclerosis, spinobulbar atrophy, prion disease, and apoptosis resulting from traumatic brain injury (TBI). In an additional embodiment, the compounds and methods of the invention are directed to providing neuroprotection, and or cerebroprotection.

The present invention also provides for a process of preparing a pharmaceutical composition, which comprises:

providing one or more double stranded chemically modified siRNA compound of the invention; and
admixing said compound with a pharmaceutically acceptable carrier.

In a preferred embodiment, the siRNA compound used in the preparation of a pharmaceutical composition is admixed with a carrier in a pharmaceutically effective dose. In a particular embodiment the chemically modified siRNA compound of the present invention is conjugated to a steroid or to a lipid or to another suitable molecule e.g. to cholesterol.

### Combination Therapy

The methods of treating the diseases disclosed herein include administering a novel chemically modified siRNA compound of the invention in conjunction or in combination with an additional RTP801 inhibitor, a substance which improves the pharmacological properties of the chemically modified siRNA compound, or an additional compound known to be effective in the treatment of a subject suffering from or susceptible to any of the hereinabove mentioned diseases and disorders, including microvascular disorder, eye disease and condition (e.g. macular degeneration), hearing impairment (including hearing loss), respiratory disorder, neurodegenerative disorder, spinal cord injury, angiogenesis- and apoptosis-related condition.

The present invention thus provides in another aspect, a pharmaceutical composition comprising a combination of a therapeutic siRNA compound of the invention together with at least one additional therapeutically active agent. By “in conjunction with” or “in combination with” is meant prior to, simultaneously or subsequent to. Accordingly, the individual components of such a combination are administered either sequentially or simultaneously from the same or separate pharmaceutical formulations.

Combination therapies comprising known treatments for treating microvascular disorders, eye disease and conditions (e.g. macular degeneration), hearing impairments (including hearing loss), respiratory disorders, neurodegenerative disorders (e.g. spinal cord injury), angiogenesis- and apoptosis-related conditions, in conjunction with the novel chemically modified siRNA compounds and therapies described herein are considered part of the current invention.

Accordingly, in another aspect of present invention, an additional pharmaceutically effective compound is administered in conjunction with the pharmaceutical composition of the invention. in treatment of conditions where inhibition of RTP801 activity is beneficial. In addition, the siRNA compounds of the invention are used in the preparation of a medicament for use as adjunctive therapy with a second therapeutically active compound to treat such conditions. Appropriate doses of known second therapeutic agents for use in combination with a chemically modified siRNA compound of the invention are readily appreciated by those skilled in the art.

In some embodiments the combinations referred to above are presented for use in the form of a single pharmaceutical formulation.

The administration of a pharmaceutical composition comprising any one of the pharmaceutically active siRNA compounds according to the invention is carried out by any of the many known routes of administration, including intravenously, intra-arterially, subcutaneously, intra-peritoneally or intra-cerebrally, as determined by a skilled practitioner. Using specialized formulations, it is possible to administer the compositions orally or via inhalation or via intranasal instillation.

By “in conjunction with” is meant that the additional pharmaceutically effective compound is administered prior to, at the same time as, or subsequent to administration of the pharmaceutical compositions of present invention. The individual components of such a combination referred to above, therefore, can be administered either sequentially or simultaneously from the same or separate pharmaceutical formulations. As is the case for the present siRNA compounds, a second therapeutic agent can be administered by any suitable route, for example, by oral, buccal, inhalation, sublingual, rectal, vaginal, transurethral, nasal, topical, percutaneous (i.e., transdermal), or parenteral (including intravenous, intramuscular, subcutaneous, and intracoronary) administration.

In some embodiments, a chemically modified siRNA compound of the invention and the second therapeutic agent are administered by the same route, either provided in a single composition as two or more different pharmaceutical compositions. However, in other embodiments, a different route of administration for the novel siRNA compound of the invention and the second therapeutic agent either is possible. Persons skilled in the art are aware of the best modes of administration for each therapeutic agent, either alone or in combination.

In various embodiments, the siRNA compounds of the present invention are the main active component in a pharmaceutical composition.

In another aspects, the present invention provides a pharmaceutical composition comprising two or more siRNA molecules for the treatment of any of the diseases and conditions mentioned herein. In some embodiments the two or more siRNA molecules or formulations comprising said molecules are admixed in the pharmaceutical composition in amounts which generate equal or otherwise beneficial activity. In certain embodiments the two or more siRNA molecules are covalently or non-covalently bound, or joined together by a nucleic acid linker of a length ranging from 2-100, preferably 2-50 or 2-30 nucleotides. In one embodiment, the two or more siRNA molecules target mRNA to RTP801. In some embodiments at least one of the two or more siRNA compounds target RTP801 mRNA. In some embodiments at least one of the siRNA compounds comprises an antisense sequence substantially identical to an antisense sequence set for the in any one of Tables A-I. In some embodiments the siRNA sense and antisense oligonucleotides are selected from sense and corresponding antisense oligonucleotides set forth in any one of Tables A-I, set forth in SEQ ID NOS:3-3624.

In some embodiments the pharmaceutical compositions of the invention further comprise one or more additional siRNA molecule, which targets one or more additional gene. In some embodiments, simultaneous inhibition of said additional gene(s) provides an additive or synergistic effect for treatment of the diseases disclosed herein.

The treatment regimen according to the invention is carried out, in terms of administration mode, timing of the administration, and dosage, so that the functional recovery of the patient from the adverse consequences of the conditions disclosed herein is improved.

### Conditions to be Treated

### Microvascular Disorders

Microvascular disorders include a broad group of conditions that primarily affect the microscopic capillaries and lymphatics and are therefore outside the scope of direct surgical intervention. Microvascular disease can be broadly grouped into the vasospastic, the vasculitis and lymphatic occlusive. Additionally, many of the known vascular conditions have a microvascular element to them.

Vasospastic Disease

Vasospastic diseases are a group of relatively common conditions where, for unknown reasons, the peripheral vasoconstrictive reflexes are hypersensitive. This results in inappropriate vasoconstriction and tissue ischemia, even to the point of tissue loss. Vasospastic symptoms are usually related to temperature or the use of vibrating machinery but may be secondary to other conditions.

Vasculitic Disease

Vasculitic diseases are those that involve a primary inflammatory process in the microcirculation. Vasculitis is usually a component of an autoimmune or connective tissue disorder and is not generally amenable to surgical treatment but requires immunosuppressive treatment if the symptoms are severe.

Lymphatic Occlusive Disease

Chronic swelling of the lower or upper limb (lymphoedema) is the result of peripheral lymphatic occlusion. This is a relatively rare condition that has a large number of causes, some inherited, some acquired. The mainstays of treatment are correctly fitted compression garments and the use of intermittent compression devices.

Microvascular Pathologies Associated with Diabetes

Diabetes is the leading cause of blindness, the number one cause of amputations and impotence, and one of the most frequently occurring chronic childhood diseases. Diabetes is also the leading cause of end-stage renal disease in the United States, with a prevalence rate of 31% compared with other renal diseases. Diabetes is also the most frequent indication for kidney transplantation, accounting for 22% of all transplantations.

In general, diabetic complications can be classified broadly as microvascular or macrovascular disease. Microvascular complications include neuropathy (nerve damage), nephropathy (kidney disease) and vision disorders (e.g. retinopathy, glaucoma, cataract and corneal disease). In the retina, glomerulus, and vasa nervorum, similar pathophysiologic features characterize diabetes-specific microvascular disease

(For further information, see Larsen: Williams Textbook of Endocrinology, 10th ed., 2003 Elsevier).

### Neuropathy

Neuropathy affects all peripheral nerves: pain fibers, motor neurons, autonomic nerves and therefore necessarily can affect all organs and systems. There are several distinct syndromes based on the organ systems and members affected, but these are by no means exclusive. A patient can have sensorimotor and autonomic neuropathy or any other combination. Despite advances in the understanding of the metabolic causes of neuropathy, treatments aimed at interrupting these pathological processes have been limited by side effects and lack of efficacy. Thus, treatments are symptomatic and do not address the underlying problems. Agents for pain caused by sensorimotor neuropathy include tricyclic antidepressants (TCAs), serotonin reuptake inhibitors (SSRIs) and antiepileptic drugs (AEDs). None of these agents reverse the pathological processes leading to diabetic neuropathy and none alter the relentless course of the illness.

### Diabetic Neuropathy

Diabetic neuropathies are neuropathic disorders (peripheral nerve damage) that are associated with diabetes mellitus. These conditions usually result from diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy and the most common form, peripheral neuropathy, which mainly affects the feet and legs. There are four factors involved in the development of diabetic neuropathy: microvascular disease, advanced glycated end products, protein kinase C, and the polyol pathway.

### Diabetic Limb Ischemia and Diabetic Foot Ulcers

Diabetes and pressure can impair microvascular circulation and lead to changes in the skin on the lower extremities, which in turn, can lead to formation of ulcers and subsequent infection. Microvascular changes lead to limb muscle microangiopathy, as well as a predisposition to develop peripheral ischemia and a reduced angiogenesis compensatory response to ischemic events. Microvascular pathology exacerbates Peripheral Vascular Disease (PVD) (or Peripheral Arterial Disease (PAD) or Lower Extremity Arterial Disease (LEAD)—a MACROvascular complication—narrowing of the arteries in the legs due to atherosclerosis. PVD occurs earlier in diabetics, is more severe and widespread, and often involves intercurrent microcirculatory problems affecting the legs, eyes, and kidneys.

Foot ulcers and gangrene are frequent comorbid conditions of PAD. Concurrent peripheral neuropathy with impaired sensation renders the foot susceptible to trauma, ulceration, and infection. The progression of PAD in diabetes is compounded by such comorbidity as peripheral neuropathy and insensitivity of the feet and lower extremities to pain and trauma. With impaired circulation and impaired sensation, ulceration and infection occur. Progression to osteomyelitis and gangrene may necessitate amputation. Persons with diabetes are up to 25 times more likely than non-diabetic persons to sustain a lower limb amputation, underscoring the need to prevent foot ulcers and subsequent limb loss (For further information, see Am. J. Surgery, 187; 5 Suppl 1, May 1, 2004).

### Coronary Microvascular Dysfunction in Diabetes

The correlation between histopathology and microcirculatory dysfunction in diabetes is well known from old experimental studies and from autopsy, where thickening of the basal membrane, perivascular fibrosis, vascular rarefication, and capillary hemorrhage are frequently found. It remains difficult to confirm these data in vivo, although a recent paper demonstrated a correlation between pathology and ocular microvascular dysfunction (Am J Physiol 2003; 285). A large amount of clinical studies, however, indicate that not only overt diabetes but also impaired metabolic control may affect coronary microcirculation (Hypert Res 2002; 25:893). Sambuceti et al (Circulation 2001; 104:1129) showed the persistence of microvascular dysfunction in patients after successful reopening of the infarct related artery, and which may explain the increased cardiovascular morbidity and mortality in these patients. There is mounting evidence from large acute reperfusion studies that morbidity and mortality are unrelated to the reopening itself of the infarct related artery, but much more dependent on the TIMI flow+/−myocardial blush (Stone 2002; Feldmann Circulation 2003). Herrmann indicated, among others, that the integrity of the coronary microcirculation is probably the most important clinical and prognostic factor in this context (Circulation 2001). The neutral effect of protection devices (no relevant change for TIMI flow, for ST resolution, or for MACE) may indicate that a functional impairment of microcirculation is the major determinant of prognosis. There is also increasing evidence that coronary microvascular dysfunction plays a major role in non-obstructive coronary artery disease (CAD). Coronary endothelial dysfunction remains a strong prognostic predictor in these patients.

Diabetic Nephropathy (Renal Dysfunction in Patients with Diabetes)

Diabetic nephropathy encompasses microalbuminuria (a microvascular disease effect), proteinuria and end stage renal disease (ESRD). Diabetes is the most common cause of kidney failure, accounting for more than 40 percent of new cases. Even when drugs and diet are able to control diabetes, the disease can lead to nephropathy and kidney failure. Most people with diabetes do not develop nephropathy that is severe enough to cause kidney failure. About 16 million people in the United States have diabetes, and about 100,000 people have kidney failure as a result of diabetes.

### Diabetic Retinopathy

According to the World Health Organization, diabetic retinopathy is the leading cause of blindness in working age adults and a leading cause of vision loss in diabetics. The American Diabetes Association reports that there are approximately 18 million diabetics in the United States and approximately 1.3 million newly diagnosed cases of diabetes in the United States each year. Prevent Blindness America and the National Eye Institute estimate that in the United States there are over 5.3 million people aged 18 or older with diabetic retinopathy.

Diabetic retinopathy is defined as the progressive dysfunction of the retinal vasculature caused by chronic hyperglycemia. Key features of diabetic retinopathy include microaneurysms, retinal hemorrhages, retinal lipid exudates, cotton-wool spots, capillary nonperfusion, macular edema and neovascularization. Associated features include vitreous hemorrhage, retinal detachment, neovascular glaucoma, premature cataract and cranial nerve palsies.

Specifically, apoptosis has been localized to glial cells such as Mueller cells and astrocytes and has been shown to occur within 1 month of diabetes in the STZ-induced diabetic rat model. The cause of these events is multi-factorial including activation of the diacylglycerol/PKC pathway, oxidative stress, and non-enzymatic glycosylation. The combination of these events renders the retina hypoxic and ultimately leads to the development of diabetic retinopathy. One possible connection between retinal ischemia and the early changes in the diabetic retina is the hypoxia-induced production of growth factors such as VEGF. The master regulator of the hypoxic response has been identified as hypoxia inducible factor-1 (HIF-1), which controls genes that regulate cellular proliferation and angiogenesis. RTP801 is responsive to hypoxia-responsive transcription factor hypoxia-inducible factor 1 (HIF-1) and is typically up-regulated during hypoxia both in vitro and in vivo in an animal model of ischemic stroke.

### Diabetic Macular Edema (DME)

Prevent Blindness America and the National Eye Institute estimate that in the United States there are over 5.3 million people aged 18 or older with diabetic retinopathy, including approximately 500,000 with DME. The CDC estimates that there are approximately 75,000 new cases of DME in the United States each year.

DME is a complication of diabetic retinopathy, a disease affecting the blood vessels of the retina. Diabetic retinopathy results in multiple abnormalities in the retina, including retinal thickening and edema, hemorrhages, impeded blood flow, excessive leakage of fluid from blood vessels and, in the final stages, abnormal blood vessel growth. This blood vessel growth can lead to large hemorrhages and severe retinal damage. When the blood vessel leakage of diabetic retinopathy causes swelling in the macula, it is referred to as DME. The principal symptom of DME is a loss of central vision. Risk factors associated with DME include poorly controlled blood glucose levels, high blood pressure, abnormal kidney function causing fluid retention, high cholesterol levels and other general systemic factors.

### Microvascular Diseases of the Kidney

The kidney is involved in a number of discreet clinicopathologic conditions that affect systemic and renal microvasculature. Certain of these conditions are characterized by primary injury to endothelial cells, such as: hemolytic-uremic syndrome (HUS) and thrombotic thrombocytopenic purpura (TTP). HUS and TTP are closely related diseases characterized by microangiopathic hemolytic anemia and variable organ impairment. Traditionally, the diagnosis of HUS is made when renal failure is a predominant feature of the syndrome, as is common in children. In adults, neurological impairment frequently predominates and the syndrome is then referred to as TTP. Thrombotic microangiopathy is the underlying pathologic lesion in both syndromes, and the clinical and laboratory findings in patients with either HUS or TTP overlap to a large extent. This has prompted several investigators to regard the two syndromes as a continuum of a single disease entity.

Pathogenesis: Experimental data strongly suggest that endothelial cell injury is the primary event in the pathogenesis of HUS/TTP. Endothelial damage triggers a cascade of events that includes local intravascular coagulation, fibrin deposition, and platelet activation and aggregation. The end result is the histopathological finding of thrombotic microangiopathy common to the different forms of the HUS/TTP syndrome. If HUS/TTP is left untreated, the mortality rate approaches 90%. Supportive therapy—including dialysis, antihypertensive medications, blood transfusions, and management of neurological complications—contributes to the improved survival of patients with HUS/TTP. Adequate fluid balance and bowel rest are important in treating typical HUS associated with diarrhea.

### Radiation Nephritis

The long-term consequences of renal irradiation in excess of 2500 rad can be divided into five clinical syndromes:

(i) Acute radiation nephritis occurs in approximately 40% of patients after a latency period of 6 to 13 months. It is characterized clinically by abrupt onset of hypertension, proteinuria, edema, and progressive renal failure in most cases leading to end-stage kidneys.
(ii) Chronic radiation nephritis, conversely, has a latency period that varies between 18 months and 14 years after the initial insult. It is insidious in onset and is characterized by hypertension, proteinuria, and gradual toss of renal function.
(iii) The third syndrome manifests 5 to 19 years after exposure to radiation as benign proteinuria with normal renal function.
(iv) A fourth group of patients exhibits only benign hypertension 2 to 5 years later and may have variable proteinuria. Late malignant hypertension arises 18 months to 11 years after irradiation in patients with either chronic radiation nephritis or benign hypertension. Removal of the affected kidney reversed the hypertension. Radiation-induced damage to the renal arteries with subsequent Reno vascular hypertension has been reported.
(v) A syndrome of renal insufficiency analogous to acute radiation nephritis has been observed in bone marrow transplantation (BMT) patients who were treated with total-body irradiation (TBI).

Irradiation causes endothelial dysfunction but spares vascular smooth muscle cells in the early postradiation phase. Radiation could directly damage DNA, leading to decreased regeneration of these cells and denudement of the basement membrane in the glomerular capillaries and tubules. In other kidney diseases, the microvasculature of the kidney is involved in autoimmune disorders, such as systemic sclerosis (scleroderma). Kidney involvement in systemic sclerosis manifests as a slowly progressing chronic renal disease or as scleroderma renal crisis (SRC), which is characterized by malignant hypertension and acute azotemia. It is postulated that SRC is caused by a Raynaud-like phenomenon in the kidney. Severe vasospasm leads to cortical ischemia and enhanced production of renin and angiotensin II, which in turn perpetuate renal vasoconstriction. Hormonal changes (pregnancy), physical and emotional stress, or cold temperature may trigger the Raynaud-like arterial vasospasm.

The renal microcirculation can also be affected in sickle cell disease, to which the kidney is particularly susceptible because of the low oxygen tension attained in the deep vessels of the renal medulla as a result of countercurrent transfer of oxygen along the vasa recta. The smaller renal arteries and arterioles can also be the site of thromboembolic injury from cholesterol-containing material dislodged from the walls of the large vessels.

### Retinal Microvasculopathy (AIDS Retinopathy)

Retinal microvasculopathy is seen in 100% of AIDS patients and is characterized by intraretinal hemorrhages, microaneurysms, Roth spots, cotton-wool spots (microinfarctions of the nerve fiber layer) and perivascular sheathing. The etiology of the retinopathy is unknown though it has been thought to be due to circulating immune complexes, local release of cytotoxic substances, abnormal hemorheology, and HIV infection of endothelial cells. AIDS retinopathy is now so common that cotton wool spots in a patient without diabetes or hypertension but at risk for HIV should prompt the physician to consider viral testing. There is no specific treatment for AIDS retinopathy but its continued presence may prompt a physician to reexamine the efficacy of the HIV therapy and patient compliance.

### Bone Marrow Transplantation BMT Retinopathy

Bone marrow transplantation retinopathy was first reported in 1983. It typically occurs within six months, but it can occur as late as 62 months after BMT. Risk factors such as diabetes and hypertension may facilitate the development of BMT retinopathy by heightening the ischemic microvasculopathy. There is no known age, gender or race predilection for development of BMT retinopathy. Patients present with decreased visual acuity and/or visual field deficit. Posterior segment findings are typically bilateral and symmetric. Clinical manifestations include multiple cotton wool spots, telangiectasia, microaneurysms, macular edema, hard exudates and retinal hemorrhages. Fluorescein angiography demonstrates capillary nonperfusion and dropout, intraretinal microvascular abnormalities, microaneurysms and macular edema. Although the precise etiology of BMT retinopathy has not been elucidated, it appears to be affected by several factors: cyclosporine toxicity, total body irradiation (TBI), and chemotherapeutic agents. Cyclosporine is a powerful immunomodulatory agent that suppresses graft-versus-host immune response. It may lead to endothelial cell injury and neurological side effects, and as a result, it has been suggested as the cause of BMT retinopathy. However, BMT retinopathy can develop in the absence of cyclosporine use, and cyclosporine has not been shown to cause BMT retinopathy in autologous or syngeneic bone marrow recipients. Cyclosporine does not, therefore, appear to be the sole cause of BMT retinopathy. Total body irradiation (TBI) has also been implicated as the cause of BMT retinopathy. Radiation injures the retinal microvasculature and leads to ischemic vasculopathy.

### Other Eye Disorders

### Glaucoma

Glaucoma is one of the leading causes of blindness in the world. It affects approximately 66.8 million people worldwide. At least 12,000 Americans are blinded by this disease each year (Kahn and Milton, Am J. Epidemiol. 1980, 111(6):769-76). Glaucoma is characterized by the degeneration of axons in the optic nerve head, primarily due to elevated intraocular pressure (TOP). One of the most common forms of glaucoma, known as primary open-angle glaucoma (POAG), results from the increased resistance of aqueous humor outflow in the trabecular meshwork (TM), causing IOP elevation and eventual optic nerve damage. Other main types of glaucoma are angle closure glaucoma, normal tension glaucoma and pediatric glaucoma. These are also marked by an increase of intraocular pressure (TOP), or pressure inside the eye. When optic nerve damage has occurred despite a normal IOP, this is called normal tension glaucoma. Secondary glaucoma refers to any case in which another disease causes or contributes to increased eye pressure, resulting in optic nerve damage and vision loss. Mucke (Drugs 2007, 10(1):37-41) reviews current therapeutics, including siRNA to various targets for the treatment of ocular diseases, for example, age-related macular degeneration (AMD) and glaucoma.

### Macular Degeneration

The most common cause of decreased best-corrected vision in individuals over 65 years of age in the US is the retinal disorder known as age-related macular degeneration (AMD). As AMD progresses, the disease is characterized by loss of sharp, central vision. The area of the eye affected by AMD is the Macula—a small area in the center of the retina, composed primarily of photoreceptor cells. So-called “dry” AMD, accounting for about 85%-90% of AMD patients, involves alterations in eye pigment distribution, loss of photoreceptors and diminished retinal function due to overall atrophy of cells. So-called “wet” AMD involves proliferation of abnormal choroidal vessels leading to clots or scars in the sub-retinal space. Thus, the onset of wet AMD occurs because of the formation of an abnormal choroidal neovascular network (choroidal neovascularization, CNV) beneath the neural retina. The newly formed blood vessels are excessively leaky. This leads to accumulation of subretinal fluid and blood leading to loss of visual acuity. Eventually, there is total loss of functional retina in the involved region, as a large disciform scar involving choroids and retina forms. While dry AMD patients may retain vision of decreased quality, wet AMD often results in blindness. (Hamdi & Kenney, Frontiers in Bioscience, e305-314, May 2003). CNV occurs not only in wet AMD but also in other ocular pathologies such as ocular histoplasmosis syndrome, angiod streaks, ruptures in Bruch's membrane, myopic degeneration, ocular tumors and some retinal degenerative diseases.

### Ocular Ischemic Syndrome

Patients suffering from ocular ischemic syndrome (OIS) are generally elderly, ranging in age from the 50s to 80s. Males are affected twice as commonly as females. The patient is only rarely asymptomatic. Decreased vision occurs at presentation in 90 percent of cases, and 40 percent of patients have attendant eye pain. There may also be an attendant or antecedent history of transient ischemic attacks or amaurosis fugax. Patients also have significant known or unknown systemic disease at the time of presentation. The most commonly encountered systemic diseases are hypertension, diabetes, ischemic heart disease, stroke, and peripheral vascular disease. To a lesser extent, patients manifest OIS as a result of giant cell arteritis (GCA).

Unilateral findings are present in 80 percent of cases. Common findings may include advanced unilateral cataract, anterior segment inflammation, asymptomatic anterior chamber reaction, macular edema, dilated but non-tortuous retinal veins, mid-peripheral dot and blot hemorrhages, cotton wool spots, exudates, and neovascularization of the disc and retina. There may also be spontaneous arterial pulsation, elevated intraocular pressure, and neovascularization of the iris and angle with neovascular glaucoma (NVG). While the patient may exhibit anterior segment neovascularization, ocular hypotony may occur due to low arterial perfusion to the ciliary body. Occasionally, there are visible retinal emboli (Hollenhorst plaques).

### Dry-Eye Syndrome

Dry eye syndrome is a common problem usually resulting from a decrease in the production of tear film that lubricates the eyes. Most patients with dry eye experience discomfort, and no vision loss; although in severe cases, the cornea may become damaged or infected. Wetting drops (artificial tears) may be used for treatment while lubricating ointments may help more severe cases.

### Additional Eye Disorders

Additional disorders which can be treated by the molecules and compositions of the present invention include all types of choroidal neovascularization (CNV), which occurs not only in wet AMD but also in other ocular pathologies such as ocular histoplasmosis syndrome, angiod streaks, ruptures in Bruch's membrane, myopic degeneration, ocular tumors and some retinal degenerative diseases.

### Otic Disorders

### Hearing Loss

In various embodiments, the novel chemically modified siRNA compounds of the invention are applied to various conditions of hearing loss. Without being bound by theory, the hearing loss may be due to apoptotic inner ear hair cell damage or loss (Zhang et al., Neuroscience 2003. 120:191-205; Wang et al., J. Neuroscience 23 ((24): 8596-8607), wherein the damage or loss is caused by infection, mechanical injury, loud sound (noise), aging (presbycusis), or chemical-induced ototoxicity.

By “ototoxin” in the context of the present invention is meant a substance that through its chemical action injures, impairs or inhibits the activity of the sound receptors component of the nervous system related to hearing, which in turn impairs hearing (and/or balance). In the context of the present invention, ototoxicity includes a deleterious effect on the inner ear hair cells. Ototoxins include therapeutic drugs including antineoplastic agents, salicylates, loop-diuretics; quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. Preferably a therapeutically effective composition comprising the novel chemically modified siRNA compound of the invention is given immediately after the exposure to prevent or reduce the ototoxic effect. More preferably, treatment is provided prophylactically, either by administration of the pharmaceutical composition of the invention prior to or concomitantly with the ototoxic pharmaceutical or the exposure to the ototoxin.

Incorporated herein by reference are chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210) relating to description and diagnosis of hearing and balance impairments.

Accordingly, in one aspect the present invention provides a method, novel chemically modified siRNA compounds and pharmaceutical compositions for treating a mammal, preferably human, to prevent, reduce, or treat a hearing impairment, disorder or imbalance, preferably an ototoxin-induced hearing condition, by administering to a mammal in need of such treatment a chemically modified siRNA compound of the invention. One embodiment of the invention is a method for treating a hearing disorder or impairment wherein the ototoxicity results from administration of a therapeutically effective amount of an ototoxic pharmaceutical drug. Typical ototoxic drugs are chemotherapeutic agents, e.g. antineoplastic agents, and antibiotics. Other possible candidates include loop-diuretics, quinines or a quinine-like compound, and salicylate or salicylate-like compounds.

Ototoxicity is a dose-limiting side effect of antibiotic administration. From 4 to 15% of patients receiving 1 gram per day for greater than 1 week develop measurable hearing loss, which slowly becomes worse and can lead to complete permanent deafness if treatment continues. Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2, and the like that are known to have serious toxicity, particularly ototoxicity and nephrotoxicity, which reduce the usefulness of such antimicrobial agents (see Goodman and Gilman's The Pharmacological Basis of Therapeutics, 6th ed., A. Goodman Gilman et al., eds; Macmillan Publishing Co., Inc., New York, pp. 1169-71 (1980)).

Ototoxicity is also a serious dose-limiting side-effect for anti-cancer agents. Ototoxic neoplastic agents include but are not limited to vincristine, vinblastine, cisplatin and cisplatin-like compounds and taxol and taxol-like compounds. Cisplatin-like compounds include carboplatin (Paraplatin®), tetraplatin, oxaliplatin, aroplatin and transplatin inter alia and are platinum based chemotherapeutics.

Diuretics with known ototoxic side-effect, particularly “loop” diuretics include, without being limited to, furosemide, ethacrylic acid, and mercurials.

Ototoxic quinines include but are not limited to synthetic substitutes of quinine that are typically used in the treatment of malaria.

Salicylates, such as aspirin, are the most commonly used therapeutic drugs for their anti-inflammatory, analgesic, anti-pyretic and anti-thrombotic effects. Unfortunately, they too have ototoxic side effects. They often lead to tinnitus (“ringing in the ears”) and temporary hearing loss. Moreover, if the drug is used at high doses for a prolonged time, the hearing impairment can become persistent and irreversible.

In some embodiments a siRNA compound of the invention is co-administered with an ototoxin. For example, an improved method is provided for treatment of infection of a mammal by administration of an aminoglycoside antibiotic, the improvement comprising administering a therapeutically effective amount of one or more chemically modified siRNAs compounds of the invention which down-regulate expression of RTP801, to the subject in need of such treatment to reduce or prevent ototoxin-induced hearing impairment associated with the antibiotic. The chemically modified siRNA compounds of the invention are preferably administered locally within the inner ear.

The methods, chemically modified siRNA compounds and pharmaceutical and compositions of the present invention are also effective in the treatment of acoustic trauma or mechanical trauma, preferably acoustic or mechanical trauma that leads to inner ear hair cell loss. With more severe exposure, injury can proceed from a loss of adjacent supporting cells to complete disruption of the organ of Corti. Death of the sensory cell can lead to progressive Wallerian degeneration and loss of primary auditory nerve fibers. The siRNA compounds of the invention are useful in treating acoustic trauma caused by a single exposure to an extremely loud sound, or following long-term exposure to everyday loud sounds above 85 decibels. The siRNA compounds of the present invention are useful in treating mechanical inner ear trauma, for example, resulting from the insertion of an electronic device into the inner ear. The siRNA compounds of the present invention prevent or minimize the damage to inner ear hair cells associated with the operation.

Another type of hearing loss is presbycusis, which is hearing loss that gradually occurs in most individuals as they age. About 30-35 percent of adults between the ages of 65 and 75 years and 40-50 percent of people 75 and older experience hearing loss. The siRNA compounds of the present invention prevent, reduce or treat the incidence and/or severity of inner ear disorders and hearing impairments associated with presbycusis.

### Lund Diseases and Disorders

### Lung Injury and Respiratory Disorders

In various embodiments the chemically modified siRNA compounds of the invention are useful for treating or preventing the incidence or severity of acute lung injury, in particular conditions which result from ischemic/reperfusion injury or oxidative stress and for treating chronic obstructive pulmonary disease (COPD).

Non-limiting examples of acute lung injuries include acute respiratory distress syndrome (ARDS) due to coronavirus infection or endotoxins, severe acute respiratory syndrome (SARS) and ischemia reperfusion injury associated with lung transplantation.

### Chronic Obstructive Pulmonary Disease (COPD)

Chronic obstructive pulmonary disease (COPD), affects more than 16 million Americans and is the fourth highest cause of death in the United States. Cigarette smoking causes most occurrences of the debilitating disease but other environmental factors cannot be excluded (Petty T L. 2003. Clin. Cornerstone, 5-10).

Pulmonary emphysema is a major manifestation of COPD. Permanent destruction of peripheral air spaces, distal to terminal bronchioles, is the hallmark of emphysema (Tuder, et al. Am J Respir Cell Mol Biol, 29:88-97; 2003). Emphysema is also characterized by accumulation of inflammatory cells such as macrophages and neutrophils in bronchioles and alveolar structures (Petty, 2003).

The pathogenesis of emphysema is complex and multifactorial. In humans, a deficiency of inhibitors of proteases produced by inflammatory cells, such as alpha 1-antitrypsin, has been shown to contribute to protease/antiprotease imbalance, thereby favoring destruction of alveolar extracellular matrix in cigarette-smoke (CS) induced emphysema (Eriksson, S. 1964. Acta Med Scand 175:197-205. Joos, L., Pare, P. D., and Sandford, A. J. 2002. Swiss Med Wkly 132:27-37). Matrix metalloproteinases (MMPs) play a central role in experimental emphysema, as documented by resistance of macrophage metalloelastase knockout mice against emphysema caused by chronic inhalation of CS (Hautamaki, et al: Requirement for macrophage elastase for cigarette smoke-induced emphysema in mice. Science 277:2002-2004). Moreover, pulmonary overexpression of interleukin-13 in transgenic mice results in MMP- and cathepsin-dependent emphysema (Zheng, T., et al 2000. J Clin Invest 106:1081-1093). Oxidative stress and apoptosis interact and cause emphysema due to vascular endothelial growth factor blockade (Tuder et al. Am J Respir Cell Mol Biol, 29:88-97; 2003; Yokohori N, et al. Chest. 2004 February; 125(2):626-32; Aoshiba K, et al. Am J Respir Cell Mol. Biol. 2003 May; 28(5):555-62). Both reactive oxygen species (ROS) from inhaled cigarette smoke and those endogenously formed by inflammatory cells contribute to an increased intrapulmonary oxidant burden.

An additional pathogenic factor with regards to COPD pathogenesis is the observed decreased expression of VEGF and VEGFR11 in lungs of emphysematous patients (Yasunori Kasahara, et al. Am J Respir Crit. Care Med. Vol 163. pp 737-744, 2001). Moreover, inhibition of VEGF signaling using chemical VEGFR inhibitor leads to alveolar septal endothelial and then to epithelial cell apoptosis, probably due to disruption of intimate structural/functional connection of both types of cells within alveoli (Yasunori Kasahara et al. J. Clin. Invest. 106:1311-1319 (2000); Voelkel N F, Cool C D, Eur Respir J Suppl. 2003. 46:28s-32s).

In various embodiments pharmaceutical composition for treatment of respiratory disorders may be comprised of the following compound combinations: chemically modified RTP801 siRNA compound of the invention combined with a siRNA compound that targets one or more of the following genes: elastases, matrix metalloproteases, phospholipases, caspases, sphingomyelinase, and ceramide synthase.

### Acute Respiratory Distress Syndrome

Acute respiratory distress syndrome (ARDS), also known as respiratory distress syndrome (RDS) or adult respiratory distress syndrome (in contrast with infant respiratory distress syndrome, IRDS) is a serious reaction to various forms of injuries to the lung. This is the most important disorder resulting in increased permeability pulmonary edema.

ARDS is a severe lung disease caused by a variety of direct and indirect insults. It is characterized by inflammation of the lung parenchyma leading to impaired gas exchange with concomitant systemic release of inflammatory mediators which cause inflammation, hypoxemia and frequently result in failure of multiple organs. This condition is life threatening and often lethal, usually requiring mechanical ventilation and admission to an intensive care unit. A less severe form is called acute lung injury (ALI).

### Lung Cancer

Lung cancer usually develops in the cells lining the lung's air passages. It is the most lethal of all cancers worldwide, responsible for up to 3 million deaths annually. The two main types are small cell lung cancer (SCLC) and non-small cell lung cancer (NSCLC). These types are diagnosed based on the morphology of the cells. In non-small cell lung cancer, results of standard treatment are poor except for the most localized cancers. Surgery is the most potentially curative therapeutic option for this disease; radiation therapy can produce a cure in only a small number of patients and can provide palliation in most patients. Adjuvant chemotherapy may provide an additional benefit to patients with resected NSCLC. In advanced-stage disease, chemotherapy offers modest improvements in median survival, though overall survival is poor. Chemotherapy has produced short-term improvement in disease-related symptoms. Other forms of cancer in the lung are secondary tumors resulting from metastases of a primary cancer. The siRNA compounds of the present invention are useful in the treatment of lung cancer including metastases in lung tissue.

### Kidney Diseases and Disorders

The chemically modified siRNA compounds of the invention are are useful in treating or preventing various diseases and disorders that affect the kidney as disclosed herein below.

### Acute Renal Failure (ARF)

In various embodiments, the chemically modified siRNA compounds of the invention are used for treating kidney disorders, in particular acute renal failure (ARF) due to ischemia in post surgical patients, in kidney transplant patients, and acute renal failure due to chemotherapy treatment such as cisplatin administration or sepsis-associated acute renal failure.

ARF can be caused by microvascular or macrovascular disease (major renal artery occlusion or severe abdominal aortic disease). The classic microvascular diseases often present with microangiopathic hemolysis and acute renal failure occurring because of glomerular capillary thrombosis or occlusion, often with accompanying thrombocytopenia. Typical examples of these diseases include:

a) Thrombotic thrombocytopenic purpura—The classic pentad in thrombotic thrombocytopenic purpura includes fever, neurological changes, renal failure, microangiopathic hemolytic anemia and thrombocytopenia.
b) Hemolytic uremic syndrome—Hemolytic uremic syndrome is similar to thrombotic thrombocytopenic purpura but does not present with neurological changes.
c) HELLP syndrome (hemolysis, elevated liver enzymes and low platelets). HELLP syndrome is a type of hemolytic uremic syndrome that occurs in pregnant women with the addition of transaminase elevations.

Acute renal failure can present in all medical settings but is predominantly acquired in hospitals. The condition develops in 5 percent of hospitalized patients, and approximately 0.5 percent of hospitalized patients require dialysis. Over the past 40 years, the survival rate for acute renal failure has not improved, primarily because affected patients are now older and have more comorbid conditions. Infection accounts for 75 percent of deaths in patients with acute renal failure, and cardio-respiratory complications are the second most common cause of death. Depending on the severity of renal failure, the mortality rate can range from 7 percent to as high as 80 percent. Acute renal failure can be divided into three categories: Prerenal, intrinsic and postrenal ARF. Intrinsic ARF is subdivided into four categories: tubular disease, glomerular disease, vascular disease (includes microvascular) and interstitial disease.

A preferred use of the chemically modified siRNA compounds of the invention is for the prevention of acute renal failure in high-risk patients undergoing major cardiac surgery or vascular surgery. The patients at high-risk of developing acute renal failure can be identified using various scoring methods such as the Cleveland Clinic algorithm or that developed by US Academic Hospitals (QMMI) and by Veterans' Administration (CICSS).

In another preferred embodiment, the chemically modified siRNA compounds of the invention are used for treating or preventing the damage caused by nephrotoxins such as diuretics, β-blockers, vasodilator agents, ACE inhibitors, cyclosporin, aminoglycoside antibiotics (e.g. gentamicin), amphotericin B, cisplatin, radiocontrast media, immunoglobulins, mannitol, NSAIDs (e.g. aspirin, ibuprofen, diclofenac), cyclophosphamide, methotrexate, acyclovir, polyethylene glycol, β-lactam antibiotics, vancomycin, rifampicin, sulphonamides, ciprofloxacin, ranitidine, cimetidine, furosemide, thiazides, phenyloin, penicillamine, lithium salts, fluoride, demeclocycline, foscarnet, aristolochic acid.

In a further embodiment of the invention the pharmaceutical composition for treatment of ARF comprises an agent selected from the following combinations of therapeutic agents:
1) RTP801 siRNA of the invention and p53 siRNA dimers;
2) RTP801 siRNA of the invention and Fas siRNA dimers;
3) RTP801 siRNA of the invention and Bax siRNA dimers;
4) RTP801 siRNA of the invention and p53 siRNA and Fas siRNA trimers;
5) RTP801 siRNA of the invention and Bax siRNA dimers;
6) RTP801 siRNA of the invention and Noxa siRNA dimers;
7) RTP801 siRNA of the invention and Puma siRNA dimers;
8) RTP801 siRNA of the invention (REDD1) and RTP801L (REDD2) siRNA dimmers; and
9) RTP801 siRNA of the invention, Fas siRNA and any of RTP801L siRNA, p53 siRNA, Bax siRNA, Noxa siRNA or Puma siRNA to form trimers or polymers (i.e., tandem molecules which encode three siRNAs).

### Progressive Renal Disease

There is evidence that progressive renal disease is characterized by a progressive loss of the microvasculature. The loss of the microvasculature correlates directly with the development of glomerular and tubulointerstitial scarring. The mechanism is mediated in part by a reduction in the endothelial proliferative response, and this impairment in capillary repair is mediated by alteration in the local expression of both angiogenic (vascular endothelial growth factor) and anti-angiogenic (thrombospondin 1) factors in the kidney. The alteration in balance of angiogenic growth factors is mediated by both macrophage-associated cytokines (interleukin-1β) and vasoactive mediators. Finally, there is intriguing evidence that stimulation of angiogenesis and/or capillary repair may stabilize renal function and slow progression and that this benefit occurs independently of effects on BP or proteinuria (For further information see Brenner & Rector's The Kidney, 7th ed., 2004, Elsevier: Ch 33. Microvascular diseases of the kidney; and Tiwari and Vikrant, Journal of Indian Academy of Clinical Medicine 2000. 5(1):44-54).

### CNS Disease and Disorders

The chemically modified siRNA compounds of the invention are are useful in treating or preventing various diseases and disorders that affect the central nervous sytem (CNS), as disclosed herein below.

### Spinal Cord Injury

Spinal cord injury or myelopathy, is a disturbance of the spinal cord that results in loss of sensation and/or mobility. The two most common types of spinal cord injury are due to trauma and disease. Traumatic injuries are often due to automobile accidents, falls, gunshots diving accidents, and the like. Diseases that can affect the spinal cord include polio, spina bifida, tumors, and Friedreich's ataxia.

In various embodiments, the chemically modified siRNA compounds of the invention are used for treating or preventing the damage caused by spinal-cord injury especially spinal cord trauma caused by motor vehicle accidents, falls, sports injuries, industrial accidents, gunshot wounds, spinal cord trauma caused by spine weakening (such as from rheumatoid arthritis or osteoporosis) or if the spinal canal protecting the spinal cord has become too narrow (spinal stenosis) due to the normal aging process, direct damage that occur when the spinal cord is pulled, pressed sideways, or compressed, damage to the spinal-cord following bleeding, fluid accumulation, and swelling inside the spinal cord or outside the spinal cord (but within the spinal canal). The chemically modified siRNA compounds of the invention are also used for treating or preventing the damage caused by spinal-cord injury due to disease such as polio or spina bifida.

### Post Stroke Dementia

About 25% of people have dementia after a stroke with many others developing dementia over the following 5 to 10 years. In addition, many individuals experience more subtle impairments of their higher brain functions (such as planning skills and speed of processing information) and are at very high risk of subsequently developing dementia. Very small strokes in the deep parts of the brain in this process (called microvascular disease) seem to be essential in the process leading to an identified pattern of brain atrophy specific to post-stroke dementia.

### Neurodegenerative Disease

Neurodegenerative diseases are conditions in which cells of the CNS (brain and/or spinal cord) are lost. The CNS cells are not readily regenerated en masse, so excessive damage can be devastating. Neurodegenerative diseases result from deterioration of neurons or their myelin sheath, which over time leads to dysfunction and disabilities. They are crudely divided into two groups according to phenotypic effects, although these are not mutually exclusive: conditions affecting movement, such as ataxia; and conditions affecting memory and related to dementia. Non-limiting examples of neurodegenerative disease are Alzheimer's disease, Amyotrophic lateral sclerosis (ALS, also known as Lou Gehrig's Disease), Huntington's disease, Lewy body dementia and Parkinson's disease.

Another type of neurodegenerative diseases includes diseases caused by misfolded proteins, or prions. Non-limiting examples of prion diseases in humans are Creutzfeldt-Jakob disease (CJD) and variant CJD (Mad Cow Disease).

### Ischemia of the Brain

Brain injury such as trauma and stroke are among the leading causes of mortality and disability in the western world.

Traumatic brain injury (TBI) is one of the most serious reasons for hospital admission and disability in modern society. Clinical experience suggests that TBI may be classified into primary damage occurring immediately after injury, and secondary damage, which occurs during several days post injury. Current therapy of TBI is either surgical or else mainly symptomatic.

### Cerebrovascular Diseases

Cerebrovascular diseases occur predominately in the middle and late years of life. They cause approximately 200,000 deaths in the United States each year as well as considerable neurological disability. The incidence of stroke increases with age and affects many elderly people, a rapidly growing segment of the population. These diseases cause either ischemia-infarction or intracranial hemorrhage.

### Stroke

Stroke is an acute neurological injury occurring as a result of interrupted blood supply, resulting in an insult to the brain. Most cerebrovascular diseases present as the abrupt onset of focal neurological deficit. The deficit may remain fixed, or it may improve or progressively worsen, leading usually to irreversible neuronal damage at the core of the ischemic focus, whereas neuronal dysfunction in the penumbra may be treatable and/or reversible. Prolonged periods of ischemia result in frank tissue necrosis. Cerebral edema follows and progresses over the subsequent 2 to 4 days. If the region of the infarction is large, the edema may produce considerable mass effect with all of its attendant consequences.

Neuroprotective drugs are being developed in an effort to rescue neurons in the penumbra from dying, though as yet none has been proven efficacious.

Damage to neuronal tissue can lead to severe disability and death. The extent of the damage is primarily affected by the location and extent of the injured tissue. Endogenous cascades activated in response to the acute insult play a role in the functional outcome. Efforts to minimize, limit and/or reverse the damage have the great potential of alleviating the clinical consequences.

In various embodiments pharmaceutical compositions for treatment of MD, DR and spinal cord injury may be comprised of the following compound combinations:
1) The chemically modified RTP801 siRNA of the invention combined with either of VEGF siRNA, VEGF-R1 siRNA, VEGF R2 siRNA, PKC beta siRNA, MCP1 siRNA, eNOS siRNA, KLF2 siRNA, RTP801L siRNA (either physically mixed or in a tandem molecule);
2) The chemically modified RTP801 siRNA of the invention in combination with two or more siRNAs of the above list (physically mixed or in a tandem molecule encoding three siRNAs, or a combination thereof).

### Organ Transplantation

In various embodiments the chemically modified siRNA compounds of the invention are useful for treating or preventing injury, including reperfusion injury, following organ transplantation including lung, liver, heart, bone pancreas, intestine, skin, blood vessels, heart valve, bone and kidney transplantation.

The term “organ transplant” is meant to encompass transplant of any one or more of the following organs including, inter alia, lung, kidney, heart, skin, vein, bone, cartilage, liver transplantation. Although a xenotransplant can be contemplated in certain situations, an allotransplant is usually preferable. An autograft can be considered for bone marrow, skin, bone, cartilage and or blood vessel transplantation.

The siRNA compounds of the present invention are particularly useful in treating a subject experiencing the adverse effects of organ transplant, including ameliorating, treating or preventing perfusion injury.

For organ transplantation, either the donor or the recipient or both may be treated with a chemically modified siRNA compound of the present invention or pharmaceutical composition comprising at least one of the siRNA compounds of the invention. Accordingly, the present invention relates to a method of treating an organ donor or an organ recipient comprising the step of administering to the organ donor or organ recipient or both a therapeutically effective amount of at least one chemically modified siRNA compound according to the present invention.

The invention further relates to a method for preserving an organ comprising contacting the organ with an effective amount of at least one siRNA compound of the present invention. Also provided is a method for reducing or preventing injury (in particular reperfusion injury) of an organ during surgery and/or following removal of the organ from a subject comprising placing the organ in an organ preserving solution wherein the solution comprises at least one chemically modified siRNA compound according to the present invention.

### Delayed Graft Function

Delayed graft function (DGF) is the most common complication of the immediate postoperative period in renal transplantation and results in poor graft outcome (Mores et al. 1999. Nephrol. Dial. Transplant. 14(4):930-35). Although the incidence and definition of DGF vary among transplant centers, the consequences are invariable: prolonged hospital stay, additional invasive procedures, and additional cost to the patient and health-care system.

Graft rejection has been categorized into three subsets depending on the onset of graft destruction: (i) hyperacute rejection is the term applied to very early graft destruction, usually within the first 48 hours; (ii) acute rejection has an onset of several days to months or even years after transplantation and can involve humoral and/or cellular mechanisms; (iii) Chronic rejection relates to chronic alloreactive immune response.

### Acute Lung Transplant Rejection

Acute allograft rejection remains a significant problem in lung transplantation despite advances in immunosuppressive medication. Rejection, and ultimately early morbidity and mortality may result from ischemia-reperfusion (FR) injury and hypoxic injury.

### Other Diseases and Conditions

In other embodiments the chemically modified siRNA compounds of the invention are useful for treating or preventing the incidence or severity of other diseases and conditions including, without being limited to, diseases or disorders associated with uncontrolled, pathological cell growth, e.g. cancer, psoriasis, autoimmune diseases, inter alia. “Cancer” or “Tumor” refers to an uncontrolled growing mass of abnormal cells. These terms include both primary tumors, which may be benign or malignant, as well as secondary tumors, or metastases which have spread to other sites in the body. Examples of cancer-type diseases include, inter alia: carcinoma (e.g.: breast, colon and lung), leukemia such as B cell leukemia, lymphoma such as B-cell lymphoma, blastoma such as neuroblastoma and melanoma.

In further embodiments, the siRNA compounds of the invention are directed to providing neuroprotection, or to provide cerebroprotection, or to prevent and/or treat cytotoxic T cell and natural killer cell-mediated apoptosis associated with autoimmune disease and transplant rejection, or to prevent cell death of cardiac cells including heart failure, cardiomyopathy, viral infection or bacterial infection of heart, myocardial ischemia, myocardial infarct, and myocardial ischemia, coronary artery by-pass graft, or to prevent and/or treat mitochondrial drug toxicity e.g. as a result of chemotherapy or HIV therapy, to prevent cell death during viral infection or bacterial infection, or to prevent and/or treat inflammation or inflammatory diseases, inflammatory bowel disease, sepsis and septic shock, or to prevent cell death from follicle to ovocyte stages, from ovocyte to mature egg stages and sperm (for example, methods of freezing and transplanting ovarian tissue, artificial fertilization), or to preserve fertility in mammals after chemotherapy, in particular human mammals, or to prevent and/or treat, macular degeneration, or to prevent and/or treat acute hepatitis, chronic active hepatitis, hepatitis-B, and hepatitis-C, or to prevent hair loss, (e.g. hair loss due-to male-pattern baldness, or hair loss due to radiation, chemotherapy or emotional stress), or to treat or ameliorate skin damage whereby the skin damage may be due to exposure to high levels of radiation, heat, chemicals, sun, or to burns and autoimmune diseases), or to prevent cell death of bone marrow cells in myelodysplastic syndromes (MDS), to treat pancreatitis, to treat rheumatoid arthritis, psoriasis, glomerulonephritis, atherosclerosis, and graft versus host disease (GVHD), or to treat retinal pericyte apoptosis, retinal damages resulting from ischemia, diabetic retinopathy, or to treat any disease states associated with an increase of apoptotic cell death.

In other embodiments the chemically modified siRNA compounds of the invention are useful for treating or preventing the incidence or severity of other diseases and conditions in a patient. These diseases and conditions include stroke and stroke-like situations (e.g. cerebral, renal, cardiac failure), neuronal cell death, brain injuries with or without reperfusion issues.

### Oral Mucositis

Oral mucositis, also referred to as a stomatitis, is a common and debilitating side effect of chemotherapy and radiotherapy regimens, which manifests itself as erythema and painful ulcerative lesions of the mouth and throat. Routine activities such as eating, drinking, swallowing, and talking may be difficult or impossible for subjects with severe oral mucositis. Palliative therapy includes administration of analgesics and topical rinses.

### Ischemic Conditions and Reperfusion Injury

Ischemic injury is the most common clinical expression of cell injury by oxygen deprivation. The most useful models for studying ischemic injury involve complete occlusion of one of the end-arteries to an organ (e.g., a coronary artery) and examination of the tissue (e.g., cardiac muscle) in areas supplied by the artery. Complex pathologic changes occur in diverse cellular systems during ischemia. Up to a certain point, for a duration that varies among different types of cells, the injury may be amenable to repair, and the affected cells may recover if oxygen and metabolic substrates are again made available by restoration of blood flow. With further extension of the ischemic duration, cell structure continues to deteriorate, owing to relentless progression of ongoing injury mechanisms. With time, the energetic machinery of the cell—the mitochondrial oxidative powerhouse and the glycolytic pathway—becomes irreparably damaged, and restoration of blood flow (reperfusion) cannot rescue the damaged cell. Even if the cellular energetic machinery were to remain intact, irreparable damage to the genome or to cellular membranes will ensure a lethal outcome regardless of reperfusion. This irreversible injury is usually manifested as necrosis, but apoptosis may also play a role. Under certain circumstances, when blood flow is restored to cells that have been previously made ischemic but have not died, injury is often paradoxically exacerbated and proceeds at an accelerated pace—this is reperfusion injury.

In other embodiments the chemically modified siRNA compounds of the invention are useful for treating or preventing the incidence or severity of diseases associated with ischemia and lack of proper blood flow, e.g. myocardial infarction (MI) and stroke, are provided.

Reperfusion injury may occur in a variety of conditions, especially during medical intervention, including but not limited to angioplasty, cardiac surgery or thrombolysis; organ transplant; as a result of plastic surgery; during severe compartment syndrome; during re-attachment of severed limbs; as a result of multiorgan failure syndrome; in the brain as a result of stroke or brain trauma; in connection with chronic wounds such as pressure sores, venous ulcers and diabetic ulcers; during skeletal muscle ischemia or limb transplantation; as a result of mesenteric ischemia or acute ischemic bowel disease; respiratory failure as a result of lower torso ischemia, leading to pulmonary hypertension, hypoxemia, and noncardiogenic pulmonary edema; acute renal failure as observed after renal transplantation, major surgery, trauma, and septic as well as hemorrhagic shock; Sepsis; Retinal ischemia occurring as a result of acute vascular occlusion, leading to loss of vision in a number of ocular diseases such as acute glaucoma, diabetic retinopathy, hypertensive retinopathy, and retinal vascular occlusion; Cochlear ischemia; flap failure in microvascular surgery for head and neck defects; Raynaud's phenomenon and the associated digital ischemic lesions in scleroderma; spinal cord injury; vascular surgery; Traumatic rhabdomyolysis (crush syndrome); and myoglobinuria.

Further, ischemia/reperfusion may be involved in the following conditions: hypertension, hypertensive cerebral vascular disease, rupture of aneurysm, a constriction or obstruction of a blood vessel—as occurs in the case of a thrombus or embolus, angioma, blood dyscrasias, any form of compromised cardiac function including cardiac arrest or failure, systemic hypotension, cardiac arrest, cardiogenic shock, septic shock, spinal cord trauma, head trauma, seizure, bleeding from a tumor; and diseases such as stroke, Parkinson's disease, epilepsy, depression, ALS, Alzheimer's disease, Huntington's disease and any other disease-induced dementia (such as HIV induced dementia for example).

Additionally, an ischemic episode may be caused by a mechanical injury to the Central Nervous System, such as results from a blow to the head or spine. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracarnial hematoma or edema). Similarly, traumatic constriction or compression can arise from the presence of a mass of abnormal tissue, such as a metastatic or primary tumor.

### Pressure Sores

Pressure sores or pressure ulcers, are areas of damaged skin and tissue that develop when sustained pressure (usually from a bed or wheelchair) cuts off circulation to vulnerable parts of the body, especially the skin on the buttocks, hips and heels. The lack of adequate blood flow leads to ischemic necrosis and ulceration of the affected tissue. Pressure sores occur most often in patients with diminished or absent sensation or who are debilitated, emaciated, paralyzed, or long bedridden. Tissues over the sacrum, ischia, greater trochanters, external malleoli, and heels are especially susceptible; other sites may be involved depending on the patient's position.

Pressure sores are wounds which normally only heal very slowly and especially in such cases an improved and more rapid healing is of course of great importance for the patient. Furthermore, the costs involved in the treatment of patients suffering from such wounds are markedly reduced when the healing is improved and takes place more rapidly.

All the diseases and indications disclosed herein above, as well as other diseases and conditions described herein such as MI may also be treated by the compounds of this invention. Any of the above conditions can also be treated by compositions comprising any of the siRNAs disclosed in co-assigned PCT publication Nos WO 2006/023544 and WO 2007/084684. New effective therapies to treat the above mentioned diseases and disorders would be of great therapeutic value.

Additionally, the chemically modified RTP801 siRNA of the invention can be linked (covalently or non-covalently) to antibodies, in order to achieve enhanced targeting for treatment of the diseases disclosed herein, according to the following:

ARF: anti-Fas antibody (preferably neutralizing antibodies).
Macular degeneration, diabetic retinopathy, spinal cord injury: anti-Fas antibody, anti-MCP1 antibody, anti-VEGFR1 and anti-VEGFR2 antibody. The antibodies should preferably be neutralizing antibodies.

The invention has been described in an illustrative manner, and it is to be understood that the terminology used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

The present invention is illustrated in detail below with reference to examples, but is not to be construed as being limited thereto.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.) Methods of performing RT-PCR are also well known in the art.

### Cell Culture

HeLa cells (American Type Culture Collection) are cultured as described in Czauderna, et al. (Nucleic Acids Res, 2003. 31, 670-82).

Human keratinocytes are cultured at 37° C. in Dulbecco's modified Eagle medium (DMEM) containing 10% FCS.

The mouse cell line B16V (American Type Culture Collection) is cultured at 37° C. in Dulbecco's modified Eagle medium (DMEM) containing 10% FCS. Culture conditions are as described in Methods Find Exp Clin Pharmacol. 1997 May; 19(4):231-9.

In each case, the cells are subjected to the experiments as described herein at a density of about 50,000 cells per well and the double-stranded nucleic acid according to the present invention is added at a concentration of 20 nM, whereby the double-stranded nucleic acid is complexed using 1 μg/ml of a proprietary lipid (Lipofectamine™) as described below.

### Induction of Hypoxia-Like Conditions

The cells were treated with CoCl2 for inducing a hypoxia-like condition as follows: siRNA transfections were carried out in 10-cm plates (30-50% confluency) as described by Czauderna et al., 2003, supra. Briefly, siRNA were transfected by adding a preformed 10× concentrated complex of GB and lipid in serum-free medium to cells in complete medium. The total transfection volume was 10 ml. The final lipid concentration was 1.0 μg/ml; the final siRNA concentration was 20 nM unless otherwise stated. Induction of the hypoxic responses was carried out by adding CoCl₂ (100 μM) directly to the tissue culture medium 24 h before lysis.

### Example 1

### Preparation and Testing of siRNA Compounds

Selection of siRNA Oligonucleotides

Using proprietary algorithms and the known sequence of gene RTP801 (SEQ ID NO:1), the sequences of many potential siRNAs were generated. In addition to the algorithm, some of the 23-mer oligomer sequences were generated by 5′ and/or 3′ extension of the 19-mer sequences. The sequences that have been generated using this method are fully complementary to the corresponding mRNA sequence. siRNA molecules according to the above specifications are prepared essentially as described herein. Tables A-I SEQ ID. NOS:3-3624 show sense and antisense oligonucleotides useful in the preparation of siRNA compounds that target RTP801. In general, the siRNAs having specific sequences that are selected for in vitro testing are specific for both human and at least a second species such as rat or rabbit. In Tables A-I the following abbreviations are used for cross-species activity: Chn-Chinchilla; Cyn-Cynomolgus; GP-guinea-pig; Rb-rabbit; Ms-mouse; Mnk-Monkey; Chmp-chimpanzee.

The siRNA compounds of the present invention are synthesized by any methods described herein, infra.

In Vitro Data

Activity and stability results obtained with specific siRNA compounds of the present invention are provided hereinbelow. About 1.5-2×105 cells (HeLa cells or 293T cells for siRNA targeting human genes and NRK52 cells or NMUMG cells for siRNA targeting rat/mouse genes) are seeded per well in a 6 well plate (70-80% confluent).

After 24 hours (h) cells are transfected with siRNA oligos using Lipofectamine™ 2000 reagent (Invitrogene) at final concentration of 500 pM, 5 nM, 20 nM or 40 nM. The cells are incubated at 37° C. in a CO₂ incubator for 72 h.

As positive control for cell transfection, PTEN-Cy3 labeled siRNA oligos are used. As negative control for siRNA activity GFP siRNA oligos are used.

At about 72 h after transfection cells are harvested and RNA is extracted from cells.

siRNA Compounds

Tables A-I detail siRNA sequences of the present invention, which may be combined with any of the modifications/structures disclosed herein, to create novel RTP801 siRNA compounds.

The following Tables 1-2 detail in vitro activity and stability results achieved with various structures of RTP801 siRNA:

| | | | Activity | | | |
|---|---|---|---|---|---|---|
| | | | at | | Stability | Stability |
| | | | 20 nM-% | | in | in |
| siRNA- | rX2p = 2′-5′-nucs; | 3′- | target | | rat | human |
| duplex | s-sense strand | end | gene | IC50 | serum | serum |
| name | as-antisens strand | Pi | KD | (nM) | (hrs) | (hrs) |
| Redd14- | s-5′- | y | 88; | 0.36 | 10 | 10- |
| 2-5- | rGrUrGrCrCrArArCrCrUrGr | | 56 | | | good; |
| #10 | ArUrGrCrArG2prC2prU2p- | | | | | 24 |
| | 3′ (2′) | | | | | (slight |
| | as-5′- | y | | | | deg) |
| | mArGmCrUmGrCmArUmCrAmGr | | | | | |
| | GmUrUmGrGmCrAmC-3′ | | | | | |
| Redd14- | s-5′- | y | 58 | | | |
| 2Methyl | mGmUmGmCmCrArArCrCrUrGr | | | | | |
| #5 | ArUrGrCrArGrCrU-3′ | | | | | |
| | as-5′- | y | | | | |
| | rArGrCrUrGrCrArUrCrArGr | | | | | |
| | GrUrUmGmGmCmAmC-3′ | | | | | |
| Redd14- | s-5′- | y | 42 | | | |
| Sp#1 | rGrUrGrCrCrArArCrCrUrGr | | | | | |
| | ArUrGrCrArGLdCLdU-3′ | | | | | |
| | as-5′- | y | | | | |
| | rArGrCrUrGrCrArUrCrArGr | | | | | |
| | GrUrUrGrGrCrALdC-3′ | | | | | |
| Redd14- | s-5′- | y | 37; | 5.1 | 10 | 10 |
| Methyl- | mGmUmGmCmCmArArCrCrUrGr | | 25 | | | with |
| 2-5 | ArUrGrCrArGLdCLdU-3′ | | | | | deg |
| | as-5′- | y | | | | |
| | rArGrCrUrGrCrArUrCrArGr | | | | | |
| | GrUrUmGmGmCmAmC-3′ | | | | | |
| Redd14- | s-5′- | y | 94 | | | |
| Sp#1_2 | rGrUrGrCrCrArArCrCrUrGr | | | | | |
| Me-as | ArUrGrCrArGLdCLdU-3′ | | | | | |
| | as-5′- | y | | | | |
| | mAmGrCrUrGrCrArUrCrArGr | | | | | |
| | GrUrUrGrGrCrALdC-3′ | | | | | |
| Redd14- | s-5′- | y | 20; 27; | 1.57 | 24 | 24 |
| Sp#10 | rGrUrGrCrCrArArCrCrUrGr | | 29 | | | with |
| | ArUrGrCrArGLdCLdU-3′ | | | | | deg |
| | as-5′- | y | | | | |
| | mArGmCrUmGrCmArUmCrAmGr | | | | | |
| | GmUrUmGrGmCrAmC-3′ | | | | | |
| Redd14- | s-5′- | y | 96 | | | |
| Sp#1_2 | mGmUrGrCrCrArArCrCrUrGr | | | | | |
| Me-as-s | ArUrGrCrArGLdCLdU-3′ | | | | | |
| | as-5′- | y | | | | |
| | mAmGrCrUrGrCrArUrCrArGr | | | | | |
| | GrUrUrGrGrCrALdC-3′ | | | | | |
| Redd14- | s-5′- | y | 49 | | | |
| 2Me#5- | mGmUmGmCmCmArArCrCrUrGr | | | | | |
| Br-s | ArUrGrCrArG2prC2prU2p- | | | | | |
| | 3′ | | | | | |
| | as-5′- | y | | | | |
| | rArGrCrUrGrCrArUrCrArGr | | | | | |
| | GrUrUmGmGmCmAmC-3′ | | | | | |
| Redd14- | s-5′- | y | 114; | | 10 | 24 |
| 2Me#6- | mGmUmGmCmCmAAmCrCrUrGrA | | 43; 108 | | | with |
| Br-s- | rUrGrCrArG2prC2prU2p-3′ | | | | | deg |
| Sp-as | as-5′- | y | | | | |
| | mAmGrCrUrGrCrArUrCrArGr | | | | | |
| | GrUrUrGrGrCrALdC-3′ | | | | | |
| 1DDIT4_2- | s-5′- | dT | 41 | | | |
| S/ | rUrArCrUrGrUrArGrCrArUr | | | | | |
| 1DDIT4_2- | GrArArArCrA2prA2prA2p- | | | | | |
| AS | dT-3′ | | | | | |
| | AS-5′- | | | | | |
| | rUrUrUrGrUrUrUrCrArUrGr | | | | | |
| | CrUrArCrArG2prU2prA2p- | | | | | |
| | dT-3′ | | | | | |
| 2DDIT4_2- | S-5′- | dT | 75 | | | |
| S/ | rU2prA2prC2prUrGrUrArGr | | | | | |
| 1DDIT4_2- | CrArUrGrArArArCrArArA- | | | | | |
| AS | dT-3′ | | | | | |
| | AS-5′- | | | | | |
| | rUrUrUrGrUrUrUrCrArUrGr | | | | | |
| | CrUrArCrArG2prU2prA2p- | | | | | |
| | dT-3′ | | | | | |
| 1DDIT4_2- | S-5′- | 3′ + | 36 | | | 0 |
| S/ | rUrArCrUrGrUrArGrCrArUr | 5′ | | | | |
| 1DDIT4_2- | GrArArArCrA2prA2prA2p- | | | | | |
| AS- | 3′ | | | | | |
| Blunt | AS-5′- | | | | | |
| | rUrUrUrGrUrUrUrCrArUrGr | | | | | |
| | CrUrArCrArG2prU2prA2p- | | | | | |
| | 3′ | | | | | |
| 2DDIT4_2- | S-5′- | 3′ + | 77 | | | 0 |
| S/ | rU2prA2prC2pUrGrUrArGrC | 5′ | | | | |
| 1DDIT4_2- | rArUrGrArArArCrArArA-3′ | | | | | |
| AS- | AS-5′- | | | | | |
| Blunt | rUrUrUrGrUrUrUrCrArUrGr | | | | | |
| | CrUrArCrArG2prU2prA2p- | | | | | |
| | 3′ | | | | | |

DDIT4_2: (SEN: SEQ ID NO: 817; AS: SEQ ID NO: 1243)

TABLE 2

| | | | | | KD at 20 nM | |
|---|---|---|---|---|---|---|
| | | | | | (% residual | Stability |
| | | | | | mRNArelative | in human serum |
| Name | Sense | AS | Sense 5->3 | AS 5->3 | to CNL) | (hrs) |
| DDIT4_2_S236 | 17,18,19- | 17,18,19- | rU; rA; rC; rU; rG; rU; rA; | rU; rU; rU; rG; rU; rU; rU; | 41 | |
| | 2′-5′-bridge; | 2′-5′- | rG; rC; rA; rU; rG; rA; rA; | rC; rA; rU; rG; rC; rU; rA; | | |
| | 20-dT-3′-Pi | bridge; 20-dT-3′-Pi | rA; rC; rA2p; rA2p; rA2p; | rC; rA; rG2p; rU2p; rA2p; | | |
| | | | dT$ | dT$ | | |
| DDIT4_2_S237 | 1,2,3- | 17,18,19- | rU2p; rA2p; rC2p; rU; rG; | rU; rU; rU; rG; rU; rU; rU; | 75 | |
| | 2′-5′-bridge; | 2′-5′- | rU; rA; rG; rC; rA; rU; rG; | rC; rA; rU; rG; rC; rU; rA; | | |
| | 20-dT-3′-Pi | bridge; 20-dT-3′-Pi | rA; rA; rA; rC; rA; rA; rA; | rC; rA; rG2p; rU2p; rA2p; | | |
| | | | dT$ | dT$ | | |
| DDIT4_2_S729 | 17,18,19- | 17,18,19- | rU; rA; rC; rU; rG; rU; rA; | rU; rU; rU; rG; rU; rU; rU; | 36 | |
| | 2′-5′- | 2′-5′- | rG; rC; rA; rU; rG; rA; rA; | rC; rA; rU; rG; rC; rU; rA; | | |
| | bridge; Phosphate | bridge; Phosphate | rA; rC; rA2p; rA2p; rA2p | rC; rA; rG2p; rU2p; rA2p | | |
| DDIT4_2_S730 | 1,2,3- | 17,18,19- | rU2p; rA2p; rC2p; rU; rG; | rU; rU; rU; rG; rU; rU; rU; | 77 | |
| | 2′-5′-bridge; | 2′-5′- | rU; rA; rG; rC; rA; rU; rG; | rC; rA; rU; rG; rC; rU; rA; | | |
| | Phosphate | bridge; Phosphate | rA; rA; rA; rC; rA; rA; rA | rC; rA; rG2p; rU2p; rA2p | | |
| DDIT4_2_S2 | 17,18- | 17,18-2′- | rU; rA; rC; rU; rG; rU; rA; | rU; rU; rU; rG; rU; rU; rU; | | |
| | 2′-5′- | 5′-bridge | rG; rC; rA; rU; rG; rA; rA; | rC; rA; rU; rG; rC; rU; rA; | | |
| | bridge | | rA; rC; rA2p; rA2p; rA$ | rC; rA; rG2p; rU2p; rA$ | | |
| DDIT4_1_S650 | 18,19- | 1,2-2′- | rG; rU; rG; rC; rC; rA; rA; | mA; mG; rC; rU; rG; rC; rA; | 94 | |
| | L- | OMe-3′- | rC; rC; rU; rG; rA; rU; rG; | rU; rC; rA; rG; rG; rU; | | |
| | DNA- | Pi; 19-L- | rC; rA; rG; LdC; LdT | rU; rG; rG; rC; rA; LdC | | |
| | 3′- | DNA-3′- | | | | |
| | Pi; Phosphate | Pi; Phosphate | | | | |
| DDIT4_1_S651 | 18,19- | 1,3,5,7,9,11, | rG; rU; rG; rC; rC; rA; rA; | mA; rG; mC; rU; mG; rC; | 20 | 24 |
| | L- | 1,13,15,17, | rC; rC; rU; rG; rA; rU; rG; | mA; rU; mC; rA; rnG; rG; | | |
| | DNA- | 19-2′- | rC; rA; rG; LdC; LdT | mU; rU; mG; rG; mC; | | |
| | 3′- | OMe-3′- | | rA; mC | | |
| | Pi; Phosphate | Pi; Phosphate | | | | |
| DDIT4_1_S654 | 1,2-2′- | 1,2-2′- | mG; mU; rG; rC; rC; rA; rA; | mA; mG; rC; rU; rG; rC; rA; | 42 | |
| | OMe- | OMe-3′- | rC; rC; rU; rG; rA; rU; rG; | rU; rC; rA; rG; rG; rU; | | |
| | 3′- | Pi; 19-L- | rC; rA; rG; LdC; LdT | rU; rG; rG; rC; rA; LdC | | |
| | Pi; 18,19- | DNA-3′- | | | | |
| | L-DNA-3′- | Pi; Phosphate | | | | |
| | Pi; Phosphate | | | | | |
| DDIT4_1_S8 | 1,2-2′- | 1,2-2′- | mG; mU; rG; rC; rC; rA; rA; | mA; mG; rC; rU; rG; rC; rA; | 96 | |
| | OMe- | OMe-3′- | rC; rC; rU; rG; rA; rU; rG; | rU; rC; rA; rG; rG; rU; | | |
| | 3′- | Pi; 19-L- | rC; rA; rG; LdC; LdT$ | rU; rG; rG; rC; rA; LdC$ | | |
| | Pi; 18,19- | DNA-3′-Pi | | | | |
| | L-DNA-3′-Pi | | | | | |
| DDIT4_1_S9 | 1,2,3,4, | 15,16,17,18, | mG; mU; mG; mC; mC; | rA; rG; rC; rU; rG; rC; rA; | 49 | |
| | 5,6-2′- | 19-2′- | mA; rA; rC; rC; rU; rG; rA; | rU; rC; rA; rG; rG; rU; rU; | | |
| | OMe- | OMe-3′-Pi | rU; rG; rC; rA; rG2p; rC2p; | mG; mG; mC; mA; mC$ | | |
| | 3′- | | rU$ | | | |
| | Pi; 17,18- | | | | | |
| | 2′-5′-bridge | | | | | |
| DDIT4_1_S653 | 1,2,3,4, | 1,2-2′-OMe-3′-Pi; | mG; mU; mG; mC; mC; | mA; mG; rC; rU; rG; rC; rA; | 67 | |
| | 5,6-2′- | 19-L-DNA-3′-Pi; | mA; rA; rC; rC; rU; rG; rA; | rU; rC; rA; rG; rG; rU; | | |
| | OMe- | Phosphate | rU; rG; rC; rA; rG2p; rC2p; | rU; rG; rG; rC; rA; LdC | | |
| | 3′-Pi; | | rU2p | | | |
| | 17,18, 19-2′-5′-bridge; | | | | | |
| | Phosphate | | | | | |
| DDIT4_1_S652 | 17,18,19- | 1,3,5,7,9,11, | rG; rU; rG; rC; rC; rA; rA; | mA; rG; mC; rU; mG; rC; | 56 | 10 |
| | 2′-5′- | 13,15,17, | rC; rC; rU; rG; rA; rU; rG; | mA; rU; mC; rA; mG; rG; | | |
| | bridge; | 19-2′- | rC; rA; rG2p; rC2p; rU2p | mU; rU; mG; rG; mC; | | |
| | Phosphate | OMe-3′- | | rA; mC | | |
| | | Pi; Phosphate | | | | |
| DDIT4_1_S4 | 1,2,3,4, | 15,16,17,18, | mG; mU; mG; mC; mC; rA; | rA; rG; rC; rU; rG; rC; rA; | 58 | |
| | 5-2′- | 19-2′- | rA; rC; rC; rU; rG; rA; rU; | rU; rC; rA; rG; rG; rU; rU; | | |
| | OMe- | OMe-3′-Pi | rG; rC; rA; rG; rC; rU$ | mG; mG; mC; mA; mC$ | | |
| | 3′-Pi | | | | | |
| DDIT4_1_S648 | 18,19- | 19-L- | rG; rU; rG; rC; rC; rA; rA; | rA; rG; rC; rU; rG; rC; rA; | 42 | |
| | L- | DNA-3′-Pi | rC; rC; rU; rG; rA; rU; rG; | rU; rC; rA; rG; rG; rU; rU; | | |
| | DNA-3′-Pi | | rC; rA; rG; LdC; LdT | rG; rG; rC; rA; LdC | | |
| DDIT4_1_S655 | 1,2,3,4, | 15,16,17,18, | mG; mU; mG; mC; mC; | rA; rG; rC; rU; rG; rC; rA; | 25 | 10 |
| | 5,6-2′- | 19-2′- | mA; rA; rC; rC; rU; rG; rA; | rU; rC; rA; rG; rG; rU; rU; | | |
| | OMe- | OMe-3′- | rU; rG; rC; rA; rG; LdC; | mG; mG; mC; mA; mC | | |
| | 3′- | Pi; Phosphate | LdT | | | |
| | Pi; 18,19-L-DNA-3′- | | | | | |
| | Pi; Phosphate | | | | | |
| DDIT4_1_S1 | 17,18-2′-5′-bridge | 1,3,5,7,9,11, | rG; rU; rG; rC; rC; rA; rA; | mA; rG; mC; rU; mG; rC; | | |
| | | 13,15,17, | rC; rC; rU; rG; rA; rU; rG; | mA; rU; mC; rA; mG; rG; | | |
| | | 19-2′-OMe-3′-Pi | rC; rA; rG2p; rC2p; rU$ | mU; rU; mG; rG; mC; | | |
| | | | | rA; mC$ | | |

DDIT4_1 = Redd14 (SEN SEQ ID NO: 66, AS SEQ ID NO: 16)

Table 3 hereinbelow provides a code of the modified nucleotides/unconventional moieties ultilized in preparing the siRNA ologonucleotides of the present invention.

TABLE 3

| Code | modification |
|---|---|
| Nuc | |
| 5medG | 5-methyl-deoxyriboguanosine-3′-phosphate |
| c6Np | Amino modifier C6 (Glen Research 10-1906-xx) |
| dA | deoxyriboadenosine-3′-phosphate |
| dB | abasic deoxyribose-3′-phosphate |
| dC | deoxyribocytidine-3′-phosphate |
| dG | deoxyriboguanosine-3′-phosphate |
| dT | thymidine-3′-phosphate |
| dT$ | thymidine (no phosphate) |
| enaA$ | ethylene-bridged nucleic acid adenosine (no phosphate) |
| enaC | ethylene-bridged nucleic acid cytidine 3′ phosphate |
| enaG | ethylene-bridged nucleic acid guanosine 3′ phosphate |
| enaT | ethylene-bridged nucleic acid thymidine 3′ phosphate |
| iB | inverted deoxyabasic |
| LdA | L-deoxyriboadenosine-3′-phosphate (mirror image dA) |
| LdA$ | L-deoxyriboadenosine (no phosphate) (mirror image dA) |
| LdC | L-deoxyribocyt0idine-3′-phosphate (mirror image dC) |
| LdC$ | L-deoxyribocytidine (no phosphate) (mirror image dC) |
| LdG | L-deoxyriboguanosine-3′-phosphate (mirror image dG) |
| LdT | L-deoxyribothymidine-3′-phosphate (mirror image dT) |
| LdT$ | L-deoxyribothymidine (no phosphate) (mirror image dT) |
| mA | 2′-O-methyladenosine-3′-phosphate |
| mA$ | 2′-O-methyladenosine (no phosphate) |
| mC | 2′-O-methylcytidine-3′-phosphate |
| mC$ | 2′-O-methylcytidine (no 3′-phosphate) |
| mG | 2′-O-methylguanosine-3′-phosphate |
| mG$ | 2′-O-methylguanosine (no phosphate) |
| mU | 2′-O-methyluridine-3′-phosphate |
| mU$ | 2′-O-methyluridine (no phosphate) |
| rA | riboadenosine-3′-phosphate |
| rA$ | riboadenosine (no phosphate) |
| rC | ribocytidine-3′-phosphate |
| rC$ | ribocytidine (no phosphate) |
| rC2p | ribocytidine-2′-phosphate |
| rG | riboguanosine-3′-phosphate |
| rG2p | riboguanosine-2′-phosphate |
| rU | ribouridine-3′-phosphate |
| rU$ | ribouridine (no phosphate) |
| rU2p | ribouridine-2′-phosphate |

### Example 2

### Model Systems of Acute Renal Failure (ARF)

ARF is a clinical syndrome characterized by rapid deterioration of renal function that occurs within days. Without being bound by theory the acute kidney injury may be the result of renal ischemia-reperfusion injury such as renal ischemia-reperfusion injury in patients undergoing major surgery such as major cardiac surgery. The principal feature of ARF is an abrupt decline in glomerular filtration rate (GFR), resulting in the retention of nitrogenous wastes (urea, creatinine). Recent studies, support that apoptosis in renal tissues is prominent in most human cases of ARF. The principal site of apoptotic cell death is the distal nephron. During the initial phase of ischemic injury, loss of integrity of the actin cytoskeleton leads to flattening of the epithelium, with loss of the brush border, loss of focal cell contacts, and subsequent disengagement of the cell from the underlying substratum.

Testing an active siRNA compound is performed using an animal model for ischemia-reperfusion-induced ARF.

Protection Against Ischemia-Reperfusion Induced ARF

Ischemia-reperfusion injury is induced in rats following 45 minutes bilateral kidney arterial clamp and subsequent release of the clamp to allow 24 hours of reperfusion. 12 mg/kg of siRNA compounds are injected into the jugular vein 30 minutes prior to and 4 hours following the clamp. ARF progression is monitored by measurement of serum creatinine levels before (baseline) and 24 hrs post surgery. At the end of the experiment, the rats are perfused via an indwelling femoral line with warm PBS followed by 4% paraformaldehyde. The left kidneys are surgically removed and stored in 4% paraformaldehyde for subsequent histological analysis. Acute renal failure is frequently defined as an acute increase of the serum creatinine level from baseline. An increase of at least 0.5 mg per dL or 44.2 μmol per L of serum creatinine is considered as an indication for acute renal failure. Serum creatinine is measured at time zero before the surgery and at 24 hours post ARF surgery.

siRNA compounds of the present invention are tested in the above model system and found to be protective against ischemia reperfusion.

Further, testing active siRNA for treating ARF may also be done using sepsis-induced ARF.

Two predictive animal models of sepsis-induced ARF are described by Miyaji et al., 2003, Ethyl pyruvate decreases sepsis-induced acute renal failure and multiple organ damage in aged mice, Kidney Int. November; 64(5):1620-31. These two models are lipopolysaccharide administration and cecal ligation puncture in mice, preferably in aged mice.

### Example 3

### Model Systems of Pressure Sores or Pressure Ulcers

Pressure sores or pressure ulcers including diabetic ulcers, are areas of damaged skin and tissue that develop when sustained pressure (usually from a bed or wheelchair) cuts off circulation to vulnerable parts of the body, especially the skin on the buttocks, hips and heels. The lack of adequate blood flow leads to ischemic necrosis and ulceration of the affected tissue. Pressure sores occur most often in patients with diminished or absent sensation or who are debilitated, emaciated, paralyzed, or long bedridden. Tissues over the sacrum, ischia, greater trochanters, external malleoli, and heels are especially susceptible; other sites may be involved depending on the patient's situation.

Testing the active inhibitors of the invention (such as siRNA compounds) for treating pressure sore, ulcers and similar wounds is performed in a mouse model described in Reid et al., J Surgical Research. 116:172-180, 2004.

An additional rabbit model is described by Mustoe et al, JCI, 1991. 87(2):694-703; Ahn and Mustoe, Ann Pl Surg, 1991. 24(1):17-23, and is used for testing the siRNA compounds of the invention. siRNA compounds of the present invention are tested in animal models where it is shown that these siRNA compounds treat and prevent pressure sores and ulcers.

### Example 4

### Model Systems of Chronic Obstructive Pulmonary Disease (COPD)

Chronic obstructive pulmonary disease (COPD) is characterized mainly by emphysema, which is permanent destruction of peripheral air spaces, distal to terminal bronchioles. Emphysema is also characterized by accumulation of inflammatory cells such as macrophages and neutrophils in bronchioles and alveolar structures. Emphysema and chronic bronchitis may occur as part of COPD or independently.

Testing the active inhibitors of the invention (such as siRNA) for treating COPD/emphysema/chronic bronchitis is performed in animal models such as those disclosed as follows:

Starcher and Williams, 1989. Lab. Animals, 23:234-240; Peng, et al., 2004; Am J Respir Crit Care Med, 169:1245-1251; Jeyaseelan et al., 2004. Infect. Immunol, 72: 7247-56. Additional models are described in PCT patent publication WO 2006/023544 assigned to the assignee of the present application, which is hereby incorporated by reference into this application.

siRNA compounds of the present invention is tested in these animal models, which show that these siRNA compounds treat and/or prevent emphysema, chronic bronchitis and COPD.

### Example 5

### Model Systems of Spinal Cord Injury

Spinal cord injury, or myelopathy, is a disturbance of the spinal cord that results in loss of sensation and/or mobility. The two common types of spinal cord injury are due to trauma and disease. Traumatic injury can be due to automobile accidents, falls, gunshot, diving accidents inter alia, and diseases which can affect the spinal cord include polio, spina bifida, tumors and Friedreich's ataxia.

Rats are injected with two different doses of Cy3 labeled siRNA (1 μg/μl, 10 μg/μl) and are left for 1 and 3 days before sacrifice. Histological analyses indicate that many long filamentous profiles take up the labeled siRNA as well as other processes and cell bodies. Immunostaining with antibodies to MAP2 identifies uptake of label into dendrites and into cell bodies of neurons including motorneurons. Staining with other antibodies specific to astrocytes or macrophages reveals lower uptake of Cy3 labeled siRNA as compared to neurons. These results indicate that siRNA molecules injected to the injured spinal-cord will reach the cell body and dendrites of neurons including motorneurons.

siRNA compounds of the present invention are tested in this animal model, which shows that these siRNA compounds promote functional recovery following spinal cord injury and thus may be used to treat spinal cord injury.

### Example 6

### Model Systems of Glaucoma

Testing the active inhibitors of the invention for treating or preventing glaucoma is done in the animal model for example as described by Pease et al., J. Glaucoma, 2006, 15(6):512-9 (Manometric calibration and comparison of TonoLab and TonoPen tonometers in rats with experimental glaucoma and in normal mice).

siRNA compounds of the present invention are tested in this animal model where it is demonstrated that these siRNA compounds treat and/or prevent glaucoma.

### Example 7

### Model Systems of Ischemia/Reperfusion Injury Following Lung Transplantation in Rats

Testing the active inhibitors of the invention for treating or preventing ischemia/reperfusion injury or hypoxic injury following lung transplantation is done in one or more of the experimental animal models, for example as described by Mizobuchi et al., 2004. J. Heart Lung Transplant, 23:889-93; Huang, et al., 1995. J. Heart Lung Transplant. 14: S49; Matsumura, et al., 1995. Transplantation 59: 1509-1517; Wilkes, et al., 1999. Transplantation 67:890-896; Naka, et al., 1996. Circulation Research, 79: 773-783.

siRNA compounds of the present invention are tested in these animal models, which show that these siRNA compounds treat and/or prevent ischemia-reperfusion injury following lung transplantation and thus may be used in conjunction with transplant surgery.

### Example 8

### Model Systems of Acute Respiratory Distress Syndrome

Testing the active inhibitors of the invention for treating acute respiratory distress syndrome is done in the animal model as described by Chen et al (J Biomed Sci. 2003; 10(6 Pt 1):588-92. siRNA compounds of the present invention are tested in this animal model which shows that these siRNAs treat and/or prevent acute respiratory distress syndrome and thus may be used to treat this condition.

### Example 9

### Model Systems of Hearing Loss Conditions

(i) Distribution of Cy3-PTEN siRNA in the Cochlea Following Local Application to the Round Window of the Ear

A solution of 1 μg/100 μl of Cy3-PTEN siRNA (total of 0.3-0.4 μg) PBS is applied to the round window of chinchillas. The Cy3-labelled cells within the treated cochlea are analyzed 24-48 hours post siRNA round window application after sacrifice of the chinchillas. The pattern of labeling within the cochlea is similar following 24 h and 48 h and includes labeling in the basal turn of cochlea, in the middle turn of cochlea and in the apical turn of cochlea. Application of Cy3-PTEN siRNA onto scala tympani reveals labeling mainly in the basal turn of the cochlea and the middle turn of the cochlea. The Cy3 signal persists to up to 15 days after the application of the Cy3-PTEN siRNA. The siRNA compounds of the invention are tested in this animal model which shows that there is significant penetration of these siRNA compounds to the basal, middle and apical turns of the cochlea, and that these compounds may be used in the treatment of hearing loss.

### (ii) Chinchilla Model of Carboplatin-Induced or Cisplatin-Induced Cochlea Hair Cell Death

Chinchillas are pre-treated by direct administration of specific siRNA in saline to the left ear of each animal. Saline is given to the right ear of each animal as placebo. Two days following the administration of the specific siRNA compounds of the invention, the animals are treated with carboplatin (75 mg/kg i.p.) or cisplatin (intraperitoneal infusion of 13 mg/kg over 30 minutes). After sacrifice of the chinchillas (two weeks post carboplatin treatment) the % of dead cells of inner hair cells (IHC) and outer hair cells (OHC) is calculated in the left ear (siRNA treated) and in the right ear (saline treated). It is calculated that the % of dead cells of inner hair cells (IHC) and outer hair cells (OHC) is lower in the left ear (siRNA treated) than in the right ear (saline treated).

(iii) Chinchilla Model of Acoustic-Induced Cochlea Hair Cell Death

The activity of specific siRNA in an acoustic trauma model is studied in chinchilla. The animals are exposed to an octave band of noise centered at 4 kHz for 2.5 h at 105 dB. The left ear of the noise-exposed chinchillas is pre-treated (48 h before the acoustic trauma) with 30 μg of siRNA in ˜10 μL of saline; the right ear is pre-treated with vehicle (saline). The compound action potential (CAP) is a convenient and reliable electrophysiological method for measuring the neural activity transmitted from the cochlea. The CAP is recorded by placing an electrode near the base of the cochlea in order to detect the local field potential that is generated when a sound stimulus, such as click or tone burst, is abruptly turned on. The functional status of each ear is assessed 2.5 weeks after the acoustic trauma. Specifically, the mean threshold of the compound action potential recorded from the round window is determined 2.5 weeks after the acoustic trauma in order to determine if the thresholds in the siRNA-treated ear are lower (better) than the untreated (saline) ear. In addition, the amount of inner and outer hair cell loss is determined in the siRNA-treated and the control ear.

siRNA compounds of the present invention are tested in this animal model which shows that the thresholds in the siRNA-treated ear are lower (better) than in the untreated (saline) ear. In addition, the amount of inner and outer hair cell loss is lower in the siRNA-treated ear than in the control ear.

### Example 10

### Model Systems Relating to Macular Degeneration

The compounds of the present invention are tested in the following an animal model of Choroidal neovascularization (CNV). This hallmark of wet AMD is induced in model animals by laser treatment.

### A) Mouse Model

Choroidal neovascularization (CNV) induction: Choroid neovascularization (CNV), a hallmark of wet AMD, is triggered by laser photocoagulation (532 nm, 200 mW, 100 ins, 75 μm) (OcuLight GL, Iridex, Mountain View, Calif.) performed on both eyes of each mouse on day 0 by a single individual masked to drug group assignment. Laser spots are applied in a standardized fashion around the optic nerve, using a slit lamp delivery system and a cover slip as a contact lens.

### Evaluation

For evaluation, the eyes are enucleated and fixed with 4% paraformaldehyde for 30 min at 4° C. The neurosensory retina is detached and severed from the optic nerve. The remaining RPE-choroid-sclera complex is flat mounted in Immu-Mount (Vectashield Mounting Medium, Vector) and covered with a coverslip. Flat mounts are examined with a scanning laser confocal microscope (TCS SP, Leica, Germany). Vessels are visualized by exciting with blue argon laser. The area of CNV-related fluorescence is measured by computerized image analysis using the Leica TCS SP software. The summation of whole fluorescent area in each horizontal section is used as an index for the volume of CNV.

### B) Non-Human Primate Model

CNV induction: Choroidal neovascularization (CNV) is induced in male Cynomolgus monkeys by perimacular laser treatment of both eyes prior to dose administration. The approximate laser parameters were as follows: spot size: 50-100 μm diameter; laser power: 300-700 milliwatts; exposure time: 0.1 seconds.

Treatment: Immediately following laser treatment, both eyes of all animals are subjected to a single intravitreal injection. Left eye is dosed with synthetic stabilized siRNA against RTP801, whereas the contralateral eye receives PBS (vehicle).

siRNA compounds of the present invention are tested in the above animal models of macular degeneration, in which it is shown that RTP801 siRNA molecules are effective in treatment of macular degeneration.

### Example 11

### Model Systems Relating to Microvascular Disorders

The compounds of the present invention are tested in animal models of a range of microvascular disorders as described below.

1. Diabetic Retinopathy

RTP801 promotes neuronal cell apoptosis and generation of reactive oxygen species in vitro. The assignee of the current invention also found that in RTP801 knockout (KO) mice subjected to the model of retinopathy of prematurity (ROP), pathologic neovascularization NV was reduced under hypoxic conditions, despite elevations in VEGF, whereas the lack of this gene did not influence physiologic neonatal retinal NV. Moreover, in this model, lack of RTP801 was also protective against hypoxic neuronal apoptosis and hyperoxic vaso-obliteration.

### Experiment 1

Diabetes is induced in RTP801 KO and C57/129sv wildtype (WT) littermate mice by intraperitoneal injection of STZ. After 4 weeks, ERG (single white flash, 1.4×10̂4 ftc, 5 ins) is obtained from the left eye after 1 hour of dark adaptation. RVP is assessed from both eyes using the Evans-blue albumin permeation technique.

### Experiment 2

Diabetes is induced in RTP801 knockout and in control wild type mice with the matched genetic background. For diabetes induction, the mice are injected with streptozotocin (STZ 90 mg/kg/d for 2 days after overnight fast). Animal physiology is monitored throughout the study for changes in blood glucose, body weight, and hematocrit. Vehicle-injected mice serve as controls. The appropriate animals are treated by intravitreal injections of anti-RTP801 siRNA or anti-GFP control siRNA.

Retinal vascular leakage is measured using the Evans-blue (EB) dye technique on the animals. Mice have a catheter implanted into the right jugular vein prior to Evans Blue (EB) measurements. Retinal permeability measurements in both eyes of each animal follows a standard Evans-blue protocol.

Retinopathy of Prematurity

Retinopathy of prematurity is induced by exposing the test animals to hypoxic and hyperoxic conditions, and subsequently testing the effects on the retina. Results show that RTP801 KO mice are protected from retinopathy of prematurity, thereby validating the protective effect of RTP801 inhibition.

Myocardial Infarction

Myocardial infarction is induced by Left Anterior Descending artery ligation in mice, both short term and long term.

Microvascular Ischemic Conditions

Animal models for assessing ischemic conditions include:

1. Closed Head Injury (CHI)—Experimental TBI produces a series of events contributing to neurological and neurometabolic cascades, which are related to the degree and extent of behavioral deficits. CHI is induced under anesthesia, while a weight is allowed to free-fall from a prefixed height (Chen et al, J. Neurotrauma 13, 557, 1996) over the exposed skull covering the left hemisphere in the midcoronal plane.
2. Transient middle cerebral artery occlusion (MCAO)—a 90 to 120 minutes transient focal ischemia is performed in adult, male Sprague Dawley rats, 300-370 gr. The method employed is the intraluminal suture MCAO (Longa et al., Stroke, 30, 84, 1989, and Dogan et al., J. Neurochem. 72, 765, 1999). Briefly, under halothane anesthesia, a 3-O-nylon suture material coated with Poly-L-Lysine is inserted into the right internal carotid artery (ICA) through a hole in the external carotid artery. The nylon thread is pushed into the ICA to the right MCA origin (20-23 mm). 90-120 minutes later the thread is pulled off, the animal is closed and allowed to recover.
3. Permanent middle cerebral artery occlusion (MCAO)—occlusion is permanent, unilateral-induced by electrocoagulation of MCA. Both methods lead to focal brain ischemia of the ipsilateral side of the brain cortex leaving the contralateral side intact (control). The left MCA is exposed via a temporal craniotomy, as described for rats by Tamura A. et al., J Cereb Blood Flow Metab. 1981; 1:53-60. The MCA and its lenticulostriatal branch are occluded proximally to the medial border of the olfactory tract with microbipolar coagulation. The wound is sutured, and animals returned to their home cage in a room warmed at 26° C. to 28° C. The temperature of the animals is maintained all the time with an automatic thermostat.

siRNA compounds of the present invention are tested in the above animal models of microvascular conditions, in which it is shown that RTP801 siRNA molecules ameliorate the symptoms of microvascular conditions.

### Example 12

### Model Systems for Neurodegenerative Diseases and Disorders

I. Evaluating the Efficacy of Intranasal Administration of siRNA Compounds of the Present Invention in the APP Transgenic Mouse Model of Alzheimer's Disease.

Animals and Treatment. The study includes twenty-four (24) APP [V717I] transgenic mice (female), a model for Alzheimer's disease (Moechars D. et al., EMBO J. 15(6):1265-74, 1996; Moechars D. et al., Neuroscience. 91(3):819-30), aged 11 months that are randomly divided into two equal groups (Group I and Group II).

Animals are treated with intranasal administration siRNA (200-400 ug/mice, Group I) and vehicle (Group II), 2-3 times a week, during 3 months.

Termination. Mice are sacrificed; brains are dissected and process one hemisphere for histology and freeze one hemisphere for shipment.

Evaluation. The following histological analysis is performed:

1. Anti-Aβ staining and quantification (4 slides/mouse):
2. Thio S staining and quantification (4 slides/mouse):
3. CD45 staining and quantification (4 slides/mouse):
4. GFAP (astrocytosis) staining and quantification.

II. Evaluating the Efficacy of Intranasal Administration of Specific siRNAs in a BACE-Transgenic Mouse Model of Alzheimer's Disease.

Objective. The objective of this study is to test the efficacy of intranasal delivery of specific siRNA compounds of the present invention in BACE-transgenic mouse model for Alzheimer disease.

Animals and Treatment. The study includes twenty (20) BACE-1 transgenic mice (female/male), aged 4 months that are randomly divided into two equal groups. siRNA treatment is initiated at age 4 months. siRNA compounds of the present invention are administered intranasally.

### Evaluation.

1. Behavioral test. All animals are monitored and tested for behavioral changes by subjecting the animals to periodical behavioral analysis. Spatial learning and memory in the Morris water maze is used.

2. Brain biochemistry. The brains of five (5) mice in each group are subjected to biochemical analysis. Western blot analysis of BACE, APP, CTFs and Aβ is carried out. Assay for BACE enzymatic activity is performed.

3. Immunohistochemistry. The left hemibrain of five (5) mice in each group is subjected to immunohistochemical analysis. Expression levels of BACE, APP and CTF are determined.

4. Analysis of gene knockdown by qPCR are performed in the right hemibrain of five (5) mice in each group

III. Evaluating the Efficacy of Intranasal Administration of siRNA in a Mouse Model of ALS.

Objective. To examine the efficacy of siRNA compounds of the present invention in the mutant SOD1^{G93A} mouse model of ALS.

Animals and Treatment. The following experimental groups are used for studying disease progression and lifespan:

1. Group 1—Mismatch siRNA—wild-type (n=10) and SOD1^{G93A} mice (n=10)
2. Group 2—siRNA of the present invention—wild-type (n=10) and SOD1^{G93A} mice (n=10)
3. Group 3—Untreated controls—wild-type (n=10) and SOD1^{G93A} mice (n=10)

Each experimental group is sex matched (5 male, 5 female) and contain littermates from at least 3 different litters. This design reduces bias that may be introduced by using mice from only a small number of litters, or groups of mice with a larger percentage of female SOD1^{G93A} mice (since these mice live 3-4 days longer than males).

Administration of siRNA. The route of administration of the siRNA is intranasal instillation, with administration twice weekly, starting from 30 days of age.

Analysis of disease progression. Behavioral and electromyography (EMG) analysis in treated and untreated mice is performed to monitor disease onset and progression. Mice are pre-tested before start of siRNA treatment, followed by weekly assessments. All results are compared statistically. The following tests are performed:

1. Swimming tank test: this test is particularly sensitive at detecting changes in hind-limb motor function (Raoul et al., 2005. Nat. Med. 11, 423-428; Towne et al, 2008. Mol. Ther. 16: 1018-1025).
2. Electromyography: EMG assessments are performed in the gastrocnemius muscle of the hind limbs, where compound muscle action potential (CMAP) is recorded (Raoul et al., 2005. supra).
3. Body weight: The body weight of mice is recorded weekly, as there is a significant reduction in the body weight of SOD1^{G93A} mice during disease progression (Kieran et al., 2007. PNAS USA. 104, 20606-20611).

Assessment of lifespan. The lifespan in days for treated and untreated mice is recorded and compared statistically to determine whether siRNA treatment has any significant effect on lifespan. Mice are sacrificed at a well-defined disease end point, when they have lost >20% of body weight and are unable to raise themselves in under 20 seconds. All results are compared statistically.

Post mortem histopathology. At the disease end-point mice are terminally anaesthetized and spinal cord and hind-limb muscle tissue are collected for histological and biochemical analysis.

Examining motor neuron survival. Transverse sections of lumbar spinal cord are cut using a cryostat and stained with gallocyanin, a nissl stain. From these sections the number of motor neurons in the lumbar spinal cord is counted (Kieran et al., 2007. supra), to determine whether siRNA treatment prevents motor neuron degeneration in SOD1^{G93A} mice.

Examining spinal cord histopathology. Motor neuron degeneration in SOD1^{G93A} mice results in astrogliosis and activation of microglial cells. Here, using transverse sections of lumbar spinal cord the activation of astrocytes and microglial cells is examined using immunocytochemistry to determine whether siRNA treatment reduced or prevented their activation.

Examining muscle histology. Hind-limb muscle denervation and atrophy occur as a consequence of motor neuron degeneration in SOD11^{G93A} mice. At the disease end point the weight of individual hind-limb muscles (gastrocnemius, soleus, tibialis anterior, extensor digitorium longus muscles) is recorded and compared between treated and untreated mice. Muscles are then processed histologically to examine motor end plate denervation and muscle atrophy (Kieran et al., 2005. J. Cell Biol. 169, 561-567).

For further elaboration on model systems which are used to test the compounds of the present invention, see International Patent Publication Nos. WO 06/023544A2, WO 2006/035434 and WO 2007/084684A2, co-assigned or assigned to the assignee of the present invention, which are hereby incorporated by reference in their entirety.

The siRNA compounds of the present invention are tested in the above animal models of neurodegenerative conditions, in which it is shown that RTP801 siRNA molecules ameliorate the symptoms of neurodegenerative diseases.

1. A compound having the structure:

| | |
|---|---|
| 5′ (N)ₓ-Z 3′ | (antisense strand) |
| 3′ Z′-(N′)_{y}-z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
wherein each of x=y=19;
wherein (N)x comprises at least one 2′O Methyl sugar modified ribonucleotide;
wherein in (N′)y comprises a mirror nucleotide in at least one of the 3′ terminus or 3′ penultimate position; and
wherein the sequence of (N)x is set forth in any one of SEQ ID NO:16 and SEQ ID NO:1175.

2. The compound according to claim 1, wherein in (N′)y N′ at the 3′ terminus is a mirror nucleotide.

3. The compound according to claim 1, wherein in (N′)y N′ at the 3′ penultimate position is a mirror nucleotide.

4. The compound according to claim 1, wherein in (N)x the ribonucleotides alternate between 2′-O-Methyl sugar modified ribonucleotides and unmodified ribonucleotides and the ribonucleotide located at the middle of (N)x being unmodified.

5. The compound according to claim 1, wherein (N)x comprises at least five alternating unmodified ribonucleotides and 2′O methyl sugar modified ribonucleotides beginning at the 3′ end and at least nine 2′O methyl sugar modified ribonucleotides in total and each remaining N is an unmodified ribonucleotide.

6. The compound according to claim 1, wherein in (N)x 1-5 consecutive N at the 5′ terminus are 2′O Methyl sugar modified ribonucleotides and the remainder of the N are unmodified ribonucleotides.

7. A compound having the following structure:

| | |
|---|---|
| 5′ (N)ₓ-Z 3′ | (antisense strand) |
| 3′ Z′-(N′)_{y}-z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
wherein each of x and y is independently an integer between 18 and 40;
wherein (N)x comprises at least one 2′O-Methyl sugar modified ribonucleotide;
wherein in (N′)y at least two consecutive ribonucleotides at one or more termini or starting from the penultimate position at one or more termini are mirror nucleotides or 2′-5′ bridged nucleotides; and
wherein the sequence of the ribonucleotide in (N′)y is identical to a sequence of identical length of consecutive ribonucleotides in an mRNA transcribed from the RTP801 gene and the sequence of (N)x is complementary to the sequence of (N′)y.

8. The compound according to claim 7, wherein the sequence of (N)x is an antisense sequence present in any one of Tables A-H.

9. The compound according to claim 7, wherein Z and Z′ are absent.

10. The compound according to claim 7, wherein x=y=19.

11. The compound according to claim 7, wherein
in (N)x the ribonucleotides alternate between 2′-O-methyl sugar modified ribonucleotides and unmodified ribonucleotides and the ribonucleotide located at the middle position of (N)x being unmodified; and
wherein (N′)y comprises unmodified ribonucleotides in which at least two consecutive nucleotides at the 3′ terminus are L-DNA nucleotides.

12. The compound according to claim 7, wherein
in (N)x the ribonucleotides alternate between 2′-O-methyl sugar modified ribonucleotides and unmodified ribonucleotides and the ribonucleotide located at the middle position of (N)x being unmodified; and
wherein (N′)y comprises unmodified ribonucleotides in which at least two consecutive nucleotides at the 3′ terminus are joined together by a 2′-5′ bridge.

13. The compound according to claim 7, wherein
in (N)x the ribonucleotides alternate between modified ribonucleotides and unmodified ribonucleotides each modified ribonucleotide being modified so as to have a 2′-O-methyl on its sugar and the ribonucleotide located at the middle position of (N)x being unmodified; and
wherein (N′)y comprises unmodified ribonucleotides in which at least two consecutive nucleotides starting at the penultimate position from the 3′ terminus are joined together by a 2′-5′ bridge.

14. The compound according to claim 13, wherein in (N′)y three consecutive nucleotides at the 3′ terminus are joined together by a 2′-5′ bridge.

15. The compounds according to claim 1, wherein the compound is phosphorylated or unphosphorylated at one or more termini.

16. A pharmaceutical composition comprising a compound according to claim 1 and a pharmaceutically acceptable carrier.

17. (canceled)

18. The method of claim 25, wherein the eye disease is selected from the group consisting of macular degeneration, glaucoma, diabetic retinopathy and diabetic macular edema.

19. The method of claim 25, wherein the respiratory disorder is selected from the group consisting of COPD, asthma, chronic bronchitis and emphysema.

20. The method of claim 25, wherein the microvascular disorder is acute renal failure.

21. The method of claim 25, wherein the neurodegenerative disease is selected from the group consisting of Alzheimer's disease, ALS and Parkinson's disease.

22. The method of claim 25, wherein the kidney disorder is selected from the group consisting of ARF and DGF.

23-24. (canceled)

25. A method for treating or preventing the incidence or severity of a disease or condition in a subject in need thereof wherein the disease or condition and/or symptoms associated therewith is selected from the group consisting of a respiratory disorder, an eye disease, a kidney disorder, a microvascular disorder, a hearing disorder, an ischemic condition, a spinal cord injury, or a neurodegenerative disease comprising administering to the subject a pharmaceutical composition according to claim 16 in an amount effective to treat the disease or condition.
